(19) United States
(12) Patent Application Publication
(10) Pub. No.: US 2026/0345774 A1
(43) Pub. Date: Nov. 5, 2026

(54) AFFINITY BINDING ENTITIES DIRECTED TO CD70 AND METHODS OF USE THEREOF

(71) Applicant: ADICET THERAPEUTICS, INC., Redwood City, CA (US)

(72) Inventors: Blake Aftab, San Ramon, CA (US); Arun Bhat, Seattle, WA (US); Kevin Nishimoto, San Mateo, CA (US); Alexander Teague, Glen Park, CA (US); Elizabeth Speltz, Pacifica, CA (US); Gauri Lamture, San Mateo, CA (US); Beibei Ding, San Mateo, CA (US); Nitya Ramadoss, Milpitas, CA (US)

(21) Appl. No.: 19/099,758

(22) PCT Filed: Jul. 28, 2023
(86) PCT No.: PCT/US2023/029047

§ 371 Date: Jan. 29, 2025

Related U.S. Application Data
(60) Provisional application No. 63/393,787, filed on Jul. 29, 2022.

(51) Int. Cl. A61K 40/31; A61K 40/11; A61K 40/42; A61P 35/00; C07K 14/705; C07K 16/28; C12N 5/0783

(52) U.S. Cl. CPC A61K 40/31; A61K 40/11; A61K 40/421; A61K 40/4232; A61K 40/4235; A61P 35/00; C07K 14/7051; C07K 14/70578; C07K 16/2875; C12N 5/0636; C07K 2319/02; C12N 2510/00

(57) ABSTRACT

Aspects of the disclosure include compositions and methods for the treatment of CD70-expressing tumors with engineered γδ T-cells. In embodiments, the γδ T-cells comprise a chimeric antigen receptor (CAR) construct. The CAR construct can include a full-length CD27 receptor domain, a costimulatory domain, an intracellular signaling domain, a combination thereof, or all thereof. The CAR construct can contain a domain encoding for an additional polypeptide that confers advantageous functionality to the γδ T-cells.

### CROSS-REFERENCE TO RELATED APPLICATION

This application claims the benefit of priority to U.S. Provisional Application Ser. No. 63/393,787 filed on Jul. 29, 2022, the disclosure of which is expressly incorporated by reference herein in its entirety.

### FIELD OF DISCLOSURE

The present disclosure relates generally to chimeric antigen receptors (CARs) comprising an antigen binding molecule which binds CD70, polynucleotides encoding the same, host cells comprising the polynucleotides and/or CARs, and methods of treating disorders associated with CD70 expression in a patient using the same.

### BACKGROUND OF THE DISCLOSURE

In the last few years, T-cell-engaging therapies employing chimeric antigen receptor (CAR) T cells have opened up a new frontier in cancer immunotherapy (June et al., Science 2018; 359:1361-1365). One attractive target is human cluster of differentiation 70 (CD70). CD70 is a transmembrane glycoprotein and a member of the tumor necrosis factor (TNF) superfamily, and is the only known ligand for CD27. CD70 displays aberrant constitutive expression in a variety of cancers, including renal cell carcinoma, leukemia, non-small cell lung cancer, melanoma, glioblastoma, and others.

The first-generation CD70-targeted CAR utilized a full-length CD27 protein targeting B cell malignancies (Shafer et al., Blood 2011; 117:4304-4314). Subsequently, improved efficacy was demonstrated with a truncated CD27 protein (Wang et al., Clin. Cancer Res. 2017; 23:2267-2276). This CAR was brought to a phase I clinical trial for solid tumors (NCT02830724) and has been demonstrated to be effective against head and neck cancers and gliomas expressing CD70. To date, CD70-targeted CARs have been examined in the context of alpha beta (αβ) T cells derived from peripheral blood T cells. Unfortunately, however, the effective use of αβ T cells is complicated by their high alloreactive potential in general, and by their propensity to trigger cytokine release syndrome (CRS) in particular.

Gamma delta (γδ) T cells are thymus-derived lymphocytes that differ from αβ T cells in their mechanism of activation and function, as well as in their anatomical distribution. In particular, while αβ T cells function exclusively in adaptive immunity, γδ T cells are innate-like immune cells that recognize malignant cells through a repertoire of activating receptors in a MHC-independent manner, similar to Natural Killer cells (NK cells) (Welsh et al., Immunol Rev. 1997; 159:79-93). As such, and in contrast to αβ T cells, γδ T cells can potentially be used in an allogeneic setting without the risk of causing graft versus host disease (GvHD). Moreover, recent studies have suggested that engineered γδ-T cells may produce less proinflammatory cytokines than αβ-T cells, which could thereby reduce the risk of CRS in patients (Harrer et al., BMC Cancer 2017; 17(1): 551).

Despite rapid and intense development of CAR T therapies, optimal parameters for CAR T cell-target interactions that will result in in vivo and in-human efficacy are not yet well understood. Given the differences in mechanism of action and function for αβ T cells as compared to γδ T cells, demonstration of CAR function and effectiveness in the context of αβ T cells is not predictive of CAR function and effectiveness in the context of γδ T cells. Hence, the practical translation of CD70-targeted CAR T therapeutic approaches to γδ T cells is at best uncertain.

### SUMMARY OF DISCLOSURE

The present disclosure provides CD70-target CAR T γδ T cells. In one aspect, provided is a γδ T cell comprising a chimeric antigen receptor (CAR), wherein the CAR comprises a binding domain that recognizes the CD70 antigen, at least one costimulatory domain, and at least one intracellular signaling domain, and wherein the γδ T cell functionally expresses the binding domain of the CAR on the surface of the γδ T cell.

In embodiments, the binding domain comprises a full-length CD27 receptor having an amino acid sequence as set forth in SEQ ID NO: 33. In embodiments, the binding domain comprises a full-length CD27 receptor having an amino acid sequence as set forth in SEQ ID NO: 91.

In embodiments, the binding domain comprises a truncated CD27 receptor having an amino acid sequence as set forth in SEQ ID NO: 43. In embodiments, the binding domain comprises a truncated CD27 receptor having an amino acid sequence as set forth in SEQ ID NO: 92.

In embodiments, the at least one costimulatory domain is selected from TLR1, TLR2, TLR3, TLR4, TLR5, TLR6, TLR7, TLR8, TLR9, TLR10, CARD11, CD2, CD3C, CD7, CD27, CD28, CD30, CD40, CD54 (ICAM), CD70, CD80, CD83, CD86, CD134 (OX40), CD137 (4-1BB), CD278 (ICOS), FcR, LAT, NKD2C, SLP76, TRIM, and ZAP70, or combinations thereof. In embodiments, the at least one costimulatory domain is 4-1BB.

In embodiments, the at least one intracellular signaling domain is selected from CD3ζ, DAP12, LFA-1, and repeat (2-5) DAP10 YINM motifs. In embodiments, the at least one intracellular signaling domain is CD3ζ.

In some embodiments, said CAR comprises the binding domain that recognizes the CD70 antigen, followed by a 4-1BB costimulatory domain, followed in turn by a CD3ζ signaling domain.

In embodiments, said CAR is encoded by an isolated nucleic acid that is operably linked to a regulatable promoter.

In embodiments, the isolated nucleic acid that encodes the CAR encodes for one or more additional polypeptides. In embodiments, the one or more additional polypeptides is selected from the group comprising or consisting of lymphotoxin beta receptor (LTBR), a dominant negative (dn) receptor for TGF-beta or Fas, a truncated form of the human epidermal growth factor receptor (EGFRt), and membrane-bound IL-12 (mbIL-12).

In embodiments, the one or more additional polypeptides is lymphotoxin beta receptor (LTBR). In embodiments, the LTBR encoded by the isolated nucleic acid has an amino acid sequence as set forth in SEQ ID NO: 27. In embodiments, the one or more additional polypeptides is a dominant negative (dn) receptor for TGF-beta. In embodiments, the dominant negative receptor for TGF-beta is dnTGFβR2. In embodiments, the dnTGFβR2 encoded by the isolated nucleic acid has an amino acid sequence as set forth in SEQ ID NO: 23. In embodiments, the one or more additional polypeptides is a truncated form of the human epidermal growth factor receptor (EGFRt). In embodiments, the EGFRt encoded by the isolated nucleic acid has an amino acid sequence as set forth in SEQ ID NO: 19. In embodiments, the one or more additional polypeptides is a dominant negative Fas (dnFas). In embodiments, the one or more additional polypeptides is membrane-bound IL-12 (mbIL-12).

In embodiments, isolated nucleic acid encodes for at least one cleavage polypeptide sequence. In embodiments, the at least one cleavage polypeptide sequence is a 2A self-cleaving polypeptide sequence selected from T2A, P2A, E2A, and F2A self-cleaving polypeptide sequences. In embodiments, the 2A self-cleaving polypeptide sequence is P2A self-cleaving polypeptide sequence. In embodiments, the P2A self-cleaving polypeptide sequence encoded by the isolated nucleic acid has an amino acid sequence as set forth in SEQ ID NO: 8, 77, or 78.

In embodiments, the isolated nucleic acid encodes SEQ ID NO: 45, 47, 49, 51, 53, 55, 57, 59, 61, 63, 65, 67, 69, or 71. In embodiments, the isolated nucleic acid encodes SEQ ID NO: 57.

In embodiments, the γδ T cell comprises at least one disrupted endogenous gene. In embodiments, the at least one disrupted endogenous gene is cytokine inducible SH2-containing protein (CISH). In embodiments, the at least one disrupted endogenous gene is Cbl proto-oncogene B (CBL-B). In embodiments, the at least one disrupted endogenous gene is Zinc Finger Protein 91 (ZFP91). In embodiments, the at least one disrupted endogenous gene is Roquin. In embodiments, the at least one disrupted endogenous gene is CD58 and/or ICAM-1.

In embodiments, the γδ T cell exhibits in vitro and/or in vivo tumor cell killing activity against a hematological and/or solid tumor cell that exhibits cell surface expression of the CD70 antigen.

In embodiments, the tumor cell killing activity of said γδ T cell is greater than an innate level of in vitro and/or in vivo tumor cell killing activity in a control γδ T cell that does not comprise the CAR.

In embodiments, the tumor cell killing activity persists for, for about, for at least, or for at least about 6 days to 180 days after first contact with a hematological and/or solid tumor cell that exhibits cell surface expression of the CD70 antigen.

In embodiments, the γδ T cell proliferates in response to contact with the hematological and/or solid tumor cell that exhibits cell surface expression of the CD70 antigen.

In embodiments, the γδ T cell exhibits increased proliferation in response to contact with the hematological and/or solid tumor cell that exhibits cell surface expression of the CD70 antigen, as compared to a control γδ T cell that does not functionally express the binding domain of the CAR on the surface of the γδ T cell.

In embodiments, the γδ T cell proliferates in a host organism that comprises the hematological and/or solid tumor cell that exhibits cell surface expression of the CD70 antigen.

In embodiments, the γδ T cell proliferation or increased γδ T cell proliferation persists for, for about, for at least, or for at least about, 6 days to 180 days after first contact with the hematological and/or solid tumor cell that exhibits cell surface expression of the CD70 antigen.

In embodiments, the γδ T cell expresses pro-inflammatory cytokines comprising tumor necrosis factor alpha or interferon gamma after contact with the hematological and/or solid tumor cell that exhibits cell surface expression of the CD70 antigen.

In embodiments, the γδ T cell exhibits reduced, substantially reduced, essentially none, or no graft versus host response when introduced into an allogeneic host in comparison to a graft versus host response exhibited by an αβ T cell administered to an allogeneic host.

In embodiments, the γδ T cell is a δ1, a δ2, a δ3, or a δ4 γδ T cell, preferably a δ2-γδ T cell, more preferably a δ1 γδ T cell.

In one aspect of the invention, provided is a plurality of γδ T cells according to embodiments described herein.

In embodiments, the plurality comprises at least about 10⁷ γδ T cells, preferably from about 10⁸ γδ T cells to about 10¹¹ γδ T cells.

In embodiments, the plurality comprises a composition that is at least 60%, 80%, or from about 60% or 80% to about 90% or 95% δ1, δ2, δ3, or δ4 γδ T cells, preferably δ1 or δ2 γδ T cells, more preferably δ2⁻ γδ T cells, most preferably δ1 γδ T cells.

In one aspect, provided is a method of making a γδ T cell according to embodiments herein, or a plurality of γδ T cells according to embodiments herein, wherein the method comprises transfecting γδ T cell(s) with a construct comprising the isolated nucleic acid according to embodiments herein. In embodiments, the method comprises retroviral transduction. In embodiments, the method comprises ex vivo expansion of the γδ T cell(s), wherein the ex vivo expansion is performed before transfection and/or after transfection of the isolated nucleic acid.

In one aspect, provided is a pharmaceutical composition comprising a pharmaceutically acceptable excipient and a γδ T cell according to embodiments herein.

In one aspect, provided is a method of killing a hematological and/or solid tumor cell that exhibits cell surface expression of a CD70 antigen, the method comprising contacting the hematological and/or solid tumor cell with a tumor cell killing effective amount of the γδ T cell according to embodiments herein, a plurality of γδ T cells according to embodiments herein, or a pharmaceutical composition according to embodiments herein.

In embodiments, the method comprises introducing a therapeutically effective amount of the γδ T cell(s) or the pharmaceutical composition into a host organism comprising the hematological and/or solid tumor cell.

In embodiments, the method comprises introducing into a host organism comprising the hematological and/or solid tumor cell a therapeutically effective amount of the γδ T cell(s) or the pharmaceutical composition and simultaneously or sequentially administering one or more methods to elevate common gamma chain cytokine(s).

In embodiments, the administering one or more methods to elevate common gamma chain cytokine(s) comprises administering simultaneously with introducing the γδ T cell(s) or sequentially an amount of common gamma chain cytokine(s) effective to increase proliferation, cytotoxic activity, persistence, or the combination thereof of the introduced γδ T cell(s), preferably wherein the method comprises administering IL-2, more preferably wherein the method comprises administering IL-15.

In embodiments, the one or more methods to elevate common gamma chain cytokine(s) comprise administering an amount of common gamma chain cytokine(s) effective to increase proliferation, cytotoxic activity, persistence, or the combination thereof of the introduced γδ T cell(s) before and/or after introducing the γδ T cell(s).

In embodiments, the one or more methods to elevate common gamma chain cytokine(s) comprises lymphodepletion before introducing the γδ T cell(s).

In embodiments, the one or more methods to elevate common gamma chain cytokine(s) comprises secretion of one or more common gamma chain cytokine(s) from the introduced γδ T cell(s).

In embodiments, the method reduces the in vivo tumor burden in the host organism, and/or increases the mean survival time of the host organism as compared to a control organism, wherein the control organism is not treated with the γδ T cell(s) or the pharmaceutical composition.

In embodiments, the method is a method of treating cancer in a subject in need thereof.

In one aspect, provided is a method of treating cancer in a subject in need thereof, the method comprising administering a therapeutically effective amount of γδ T cells, wherein the cancer comprises hematological tumor cells that exhibit cell surface expression of CD70.

In one aspect, provided is a method of treating cancer in a subject in need thereof, the method comprising administering a therapeutically effective amount of γδ T cells, wherein the cancer comprises solid tumor cells that exhibit cell surface expression of CD70.

In one aspect, provided is a method of treating cancer in a subject in need thereof, the method comprising administering a therapeutically effective amount of γδ T cells, wherein the cancer comprises hematological and/or solid tumor cells that exhibit cell surface expression of CD70.

In some embodiments of any one of the methods of treating cancer described herein, the method may comprise performing a plurality of administrations of the γδ T cells, wherein the interval between the plurality of administrations is at least about a week, preferably at least about 2, 3, 4, 5, 6, 7, 8, or 12 weeks, and/or wherein the plurality of administrations is no more than once every 6 or 12 months.

In one aspect, provided is a method of reducing or inhibiting the graft vs. host response to an immune cell administered to a subject in need thereof, comprising administering a therapeutically effective amount of γδ T cells according to the subject invention. In embodiments, the γδ T cells may comprise a dual CAR binding to CD70 and another tumor-associated antigen, or the method may further comprise co-administering the γδ T cell according to the subject invention simultaneously or sequentially with an immune cell (e.g., a T cell or NK cell) comprising a CAR binding to another tumor associated antigen.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

FIG. 1 is a graph illustrating CD70 expression in different cancer cell lines.

FIGS. 2A-2C depict graphs illustrating in vitro cytotoxicity of Vδ1 CD27 receptor CAR T cells against various cancer cell lines.

FIG. 3 is a graph illustrating that in vitro cytotoxicity of Vδ1 CD27 receptor CAR T cells against A498 cells is not affected by the presence of soluble CD27.

FIG. 4 depicts graphs illustrating in vivo therapeutic efficacy of Vδ1 CD27 receptor CAR T cells in a subcutaneous human xenograft A498 cell NOD scid gamma (NSG) mouse model.

FIG. 5 depicts graphs illustrating in vivo therapeutic efficacy of Vδ1 CD27 receptor CAR T cells in a subcutaneous human xenograft ACHN cell NSG mouse model.

FIG. 6 depicts a series of plots illustrating the transduction efficiency of Vδ1 T cells by staining for P2A as a method for CAR detection.

FIG. 7 is a graph illustrating the gene knockout efficiency of two different guide RNAs targeting cytokine inducible SH2 containing protein (CISH) in Vδ1 T cells.

FIG. 8A is a graph illustrating Vδ1 T cell enrichment post-CISH gene knockout from two different guide RNAs.

FIG. 8B is a graph illustrating cell viability of Vδ1 T cells post CISH gene knockout from two different guide RNAs.

FIG. 9 illustrates the transduction efficiency of ADI-270 by staining for P2A as a method for CAR detection.

FIG. 10 illustrates CD70 expression in different cancer cell lines.

FIG. 11 illustrates in vitro cytotoxicity of ADI-270 against various cancer cell lines.

FIG. 12 illustrates in vitro cytotoxicity of ADI-270 in the presence of TGFβ1.

FIG. 13 illustrates in vitro proliferation of ADI-270 in the presence of TGFβ1.

FIG. 14 illustrates intracellular SMAD2/3 phosphorylation of ADI-270 in the presence of TGFβ1.

FIG. 15 illustrates ADI-270 was armored against TGF-β1-mediated transcript alterations.

FIGS. 16A-16B illustrate ADI-270 cytokine profile with proinflammatory and chemo-attractive cytokines.

FIGS. 17A-17B illustrate in vivo therapeutic efficacy of ADI-270 in a subcutaneous human xenograft A498 cell NSG mouse model.

FIGS. 18A-18B illustrate in vivo therapeutic efficacy of ADI-270 in a subcutaneous human xenograft A498 cell NSG mouse model.

FIGS. 19A-19B illustrate in vivo therapeutic efficacy of ADI-270 in a subcutaneous human xenograft HuT78 cell NSG mouse model.

FIGS. 20A-20C illustrate ADI-270 homed to the tumor, proliferates, and expresses activation markers from ex vivo tissue analysis.

FIG. 21 illustrates Host vs Graft (HvG) armoring supported improved persistence of ADI-270.

FIG. 22 illustrates the CAR-mbIL-12 constructs designs.

FIGS. 23A-23D illustrate the expansion and expression of the CAR-mbIL-12 in Vδ1 T cells.

FIG. 24 illustrates the enhanced in vitro cytotoxicity of CAR-mbIL-12 in Vδ1 T cells.

FIG. 25. illustrates in vivo therapeutic efficacy of CAR-mbIL-12 in Vδ1 T cells in a subcutaneous human xenograft Raji cell NSG mouse model.

FIGS. 26A-26B. illustrate CISH KO enhanced the in vitro cytotoxicity of Vδ1 T cells.

FIGS. 27A-27B. illustrate CBL-B KO enhanced the in vitro cytotoxicity of Vδ1 T cells.

FIGS. 28A-28B. illustrate Roquin KO enhanced the in vitro cytotoxicity of Vδ1 T cells.

FIGS. 29A-29B. illustrates CD58 or ICAM-1 KO enhanced the in vitro cell survival of Vδ1 T cells in an allogeneic MLR assay.

### DETAILED DESCRIPTION

### I. Definitions

For purposes of interpreting this specification, the following definitions will apply, and whenever appropriate, terms used in the singular will also include the plural and vice versa. In the event that any definition set forth conflicts with any document incorporated herein by reference, the definition set forth below shall control. Unless defined otherwise, all technical and scientific terms used herein have the same meaning as commonly understood by one of ordinary skill in the art to which the disclosure pertains.

“About” as used herein when referring to a measurable value such as an amount, a temporal duration, and the like, is meant to encompass variations of ±20% or ±10%, more preferably ±5%, even more preferably ±1%, and still more preferably ±0.1% from the specified value, as such variations are appropriate to perform the disclosed methods.

As used herein, “w/v” refers to the weight of the component in a given volume of solution.

“Ranges”: throughout this disclosure, various aspects of the disclosure can be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

The terms “patient,” “subject,” “individual,” and the like are used interchangeably herein, and refer to any animal amenable to the methods described herein. In certain non-limiting embodiments, the patient, subject or individual is a human.

The term “diagnosis”, or “diagnosing” as used herein refers to the process of identifying a disease, such as cancer, by its signs, symptoms, and/or results of various tests. A conclusion reached through such a process is a diagnosis. Forms of testing commonly performed include blood tests, medical imaging, urinalysis, biopsy, and the like.

As used herein, the term “agent” refers to any protein, nucleic acid molecule (including chemically modified nucleic acids), compound, antibody, small molecule, organic compound, inorganic compound, other molecule of interest, or cell (e.g., cell engineered to express a chimeric antigen receptor). Agent can include a therapeutic agent, a diagnostic agent or a pharmaceutical agent. A therapeutic or pharmaceutical agent is one that alone or together with an additional agent induces the desired response (such as inducing a therapeutic or prophylactic effect when administered to a subject, including treating a subject suffering from cancer, or other disease/condition.

The term “therapeutically effective amount”, or simply “effective amount” refers to the amount of an agent or composition (e.g., composition comprising an agent) that will elicit a biological or medical response of a tissue, system, or subject that is being sought by the researcher, veterinarian, medical doctor or other clinician. The term “therapeutically effective amount” includes that amount of an agent, or a composition comprising an agent, that, when administered, is sufficient to prevent development of, or alleviate to some extent, one or more of the signs or symptoms of the disorder or disease (e.g., tumor cell expressing CD70) being treated. The therapeutically effective amount will vary depending on the composition, the disease and its severity and the age, weight, etc., of the subject to be treated.

The term “γδ T-cells (gamma delta T-cells)” as used herein refers to a subset of T-cells that express a distinct T-cell receptor (TCR), namely γSTCR, on their surface, composed of one γ-chain and one δ-chain. The term “γδ T-cells” specifically includes all subsets of γδ T-cells, including, without limitation, Vδ1 and Vδ2, Vδ3 γδ T cells, as well as naïve, effector memory, central memory, and terminally differentiated γδ T-cells. As a further example, the term “γδ T-cells” includes Vδ4, Vδ5, Vδ7, and Vδ8 γδ T cells, as well as Vγ2, Vγ3, Vγ5, Vγ8, Vγ9, Vγ10, and Vγ11 γδ T cells. In some embodiments, the γδ T-cells are Vδ1⁻, Vδ2⁻, or Vδ1⁻ and Vδ2⁻. Compositions and methods for making and using engineered and non-engineered γδ T cells and/or sub-types thereof include, without limitation, those described in US 2016/0175358; WO 2017/197347; U.S. Pat. No. 9,499,788; US 2018/0169147; U.S. Pat. No. 9,907,820; US 2018/0125889 and US 2017/0196910, the contents of each of which are incorporated by reference for all purposes, including the said compositions and methods for making and using engineered and non-engineered γδ T cells and/or sub-types thereof. The present application further contemplates T cells, or other engineered leukocytes or lymphocytes, that express one γ-chain or one δ-chain, optionally in combination with a second polypeptide to form a functional TCR. Such engineered leukocytes or lymphocytes, that express one γ-chain or one δ-chain may be used in the methods or present in the compositions described herein.

As used herein, the term “T lymphocyte” or “T cell” refers to an immune cell that expresses or has expressed CD3 (CD3+) and a T Cell Receptor (TCR+). T cells play a central role in cell-mediated immunity. A T cell that “has expressed” CD3 and a TCR has been engineered to eliminate CD3 and/or TCR cell surface expression.

As used herein, the term “TCR” or “T cell receptor” refers to a dimeric heterologous cell surface signaling protein forming an alpha-beta or gamma-delta receptor or combinations thereof. αβTCRs recognize an antigen presented by an MHC molecule, whereas γδTCR can recognize an antigen independently of MHC presentation.

As used herein, the term “CD70” refers to a molecule that belongs to the tumor necrosis factor (TNF) ligand family, and is also known as CD27LG and TNFSF7. CD70 is a ligand for CD27 (TNFRSF27). The term CD70 can include, but is not limited to, native CD70, an isoform of CD70, or an interspecies CD70 homolog of CD70. The amino acid sequence of human CD70 (hCD70) is provided in NCBI Accession NP 001243.1 (GI: 4507605) (SEQ ID NO: 1). Residues 1-17 of SEQ ID NO: 1 correspond to the cytoplasmic region of hCD70, residues 18-38 correspond to the transmembrane region of hCD70, and residues 39-193 correspond to the extracellular region of hCD70. At least one other isoform of hCD70 is known and is identified by the UniProt identifier P32970-2. As used herein, CD70 includes human CD70 and non-human CD70 homologs, as well as variants, fragments, or post-translationally modified forms thereof, including, but not limited to, N- and O-linked glycosylated forms of CD70. CD70 proteins can further include fragments comprising all or a portion of SEQ ID NO: 1 (e.g., amino acids 39-193 of SEQ ID NO: 1, corresponding to an extracellular component of hCD70, amino acids 18-193, corresponding to a transmembrane and extracellular component of hCD70).

The term “MHC” (major histocompatibility complex) refers to a subset of genes that encodes cell-surface antigen-presenting proteins. In humans, these genes are referred to as human leukocyte antigen (HLA) genes. Herein, the abbreviations MHC or HLA are used interchangeably.

“Activation”, as used herein, refers to the state of a T cell that has been sufficiently stimulated to induce detectable cellular proliferation. Activation can also be associated with induced cytokine production, and detectable effector functions. The term “activated T cells” refers to, among other things, T cells that are undergoing cell division.

The term “antibody,” as used herein, refers to an immunoglobulin molecule which specifically binds with an antigen. Antibodies can be intact immunoglobulins derived from natural sources or from recombinant sources and can be immunoreactive portions of intact immunoglobulins. Antibodies are typically tetramers of immunoglobulin molecules. The antibodies in the present invention may exist in a variety of forms including, for example, polyclonal antibodies, monoclonal antibodies, Fv, Fab and F(ab)₂, as well as single chain antibodies and humanized antibodies (Harlow et al., 1999, In: Using Antibodies: A Laboratory Manual, Cold Spring Harbor Laboratory Press, NY; Harlow et al., 1989, In: Antibodies: A Laboratory Manual, Cold Spring Harbor, N.Y.; Houston et al., 1988, Proc. Natl. Acad. Sci. USA 85:5879-5883; Bird et al., 1988, Science 242:423-426).

The term “antigen” or “Ag” as used herein is defined as a molecule that provokes an immune response. This immune response may involve either antibody production, or the activation of specific immunologically-competent cells, or both. The skilled artisan will understand that any macromolecule, including proteins or peptides, can serve as an antigen.

The term “epitope” includes any protein determinant, lipid or carbohydrate determinant capable of specific binding to an immunoglobulin or receptor, e.g., CD27 binding domain. Epitopic determinants usually consist of active surface groupings of molecules such as amino acids, lipids or sugar side chains and usually have specific three-dimensional structural characteristics, as well as specific charge characteristics.

The term “specifically binds”, as used herein refers to a receptor (e.g., full-length CD27) which recognizes a specific molecule/ligand (e.g., CD70), but does not substantially recognize or bind other molecules in a sample. For example, a receptor that specifically binds to a molecule from one species may also bind to that molecule from one or more other species. But, such cross-species reactivity does not itself alter the classification as specific. In another example, a receptor that specifically binds to a molecule may also bind to different allelic forms of the molecule. However, such cross reactivity does not itself alter the classification as specific. In some instances, the terms “specific binding” or “specifically binding,” can be used in reference to the interaction of a protein (or a peptide) with a second chemical species, to mean that the interaction is dependent upon the presence of a particular structure (e.g., an antigenic determinant or epitope) on the chemical species; for example, a receptor recognizes and binds to a specific a structure rather than to proteins generally. If receptor is specific for epitope “A”, the presence of a molecule containing epitope A (or free, unlabeled A), in a reaction containing labeled “A” and the receptor, will reduce the amount of labeled A bound to the receptor.

In some embodiments, specific binding can be characterized by an equilibrium dissociation constant of at least about 1×10⁻⁸ M or less (e.g., a smaller KD denotes a tighter binding). Methods for determining whether two molecules specifically bind are well known in the art and include, for example, equilibrium dialysis, surface plasmon resonance, and the like.

The “costimulatory domain” in the context of a chimeric receptor, also referred to herein as a chimeric antigen receptor (CAR), of the present disclosure enhances cell proliferation, cell survival and development of memory cells for cytotoxic cells that express the chimeric receptor. The chimeric receptors of the invention may include one or more costimulatory domains selected from the costimulatory domains of proteins in the TNFR superfamily, CD28, CD137 (4-1BB), CD134 (OX40), Dap10, CD27, CD2, CD7, CD5, ICAM-1, LFA-1 (CD11a/CD18), Lck, TNFR-I, PD-1, TNFR-II, Fas, CD30, CD40, ICOS LIGHT, NKG2C, B7-H3, or combinations thereof. If the chimeric receptor includes more than one costimulatory domain, these domains may be arranged in tandem, optionally separated by a linker. The costimulatory domain is an intracellular domain that may locate between CD27 (truncated or full length) and an intracellular signaling domain in the chimeric receptor.

The term “costimulatory domain” as used herein also encompasses any modifications thereof, examples of which are described in U.S. patent application No. 20200129554; U.S. patent application No. 20200317777; WO2019010383; Li, W., et al., (2020) Immunity 53:456-470; and Li, G., et al., (2017) J Immunol 198 (1 Supplement): 198.4, the contents of each of which are incorporated herein in their entirety.

The “intracellular signaling domain” in the context of a chimeric receptor of the present disclosure transduces the effector function signal and directs the cytotoxic cell to perform its specialized function, i.e., harming and/or destroying the target cells. Examples of suitable intracellular signaling domains include, e.g., the ζ chain of the T cell receptor complex or any of its homologs, e.g., η chain, FcsRly and β chains, MB 1 (Iga) chain, B29 (Ig) chain, etc., human CD3 ζ chain, CD3 polypeptides (Δ, δ and ε), syk family tyrosine kinases (Syk, ZAP 70, etc.), src family tyrosine kinases (Lck, Fyn, Lyn, etc.) and other molecules involved in T cell transduction, such as CD2, CD5 and CD28. In embodiments, the intracellular signaling domain of a chimeric receptor may be human CD3 ζ chain, FcγRIII, FcsRI, cytoplasmic tails of Fc receptors, an immunoreceptor tyrosine-based activation motif (ITAM) bearing cytoplasmic receptors and combinations thereof.

The intracellular signaling domains may include intracellular signaling domains of several types of various other immune signaling receptors, including, but not limited to, first, second, and third generation T cell signaling proteins including CD3, B7 family costimulatory, and Tumor Necrosis Factor Receptor (TNFR) superfamily receptors (Park et al., “Are all chimeric antigen receptors created equal?” J Clin Oncol., vol. 33, pp. 651-653, 2015). Additional intracellular signaling domains include signaling domains used by NK and NKT cells (Hermanson, et al., “Utilizing chimeric antigen receptors to direct natural killer cell activity,” Front Immunol., vol. 6, p. 195, 2015) such as signaling domains of NKp30 (B7-H6) (Zhang et al., “An NKp30-based chimeric antigen receptor promotes T cell effector functions and antitumor efficacy in vivo,” J Immunol., vol. 189, pp. 2290-2299, 2012), and DAP12 (Topfer et al., “DAP12-based activating chimeric antigen receptor for NK cell tumor immunotherapy,” J Immunol., vol. 194, pp. 3201-3212, 2015), NKG2D, NKp44, NKp46, DAP10, and CD3z. Additionally intracellular signaling domains also includes signaling domains of human Immunoglobulin receptors that contain immunoreceptor tyrosine based activation motif (ITAM) such as FcgammaRI, FcgammaRIIA, FcgammaRIIC, FcgammaRIIIA, FcRL5 (Gillis et al., “Contribution of Human Fc.gamma.Rs to Disease with Evidence from Human Polymorphisms and Transgenic Animal Studies,” Front Immunol., vol. 5, p. 254, 2014).

In embodiments, the intracellular signaling domain includes a cytoplasmic signaling domain of TCR ζ, FCR γ, FcR β, CD3 γ, CD3 δ, CD3 ε, CD5, CD22, CD79a, CD79b, or CD66d. In exemplary embodiments the intracellular signaling domain in the chimeric receptor includes a cytoplasmic signaling domain of human CD3 ζ. The term “intracellular signaling domain” as used herein also encompasses any modifications thereof, examples of which are described in US Patent Application No. 2020/0317777, as well as Roda-Navarro, P., and Reyburn, H T., (2009) J Biol Chem 284(24): 16463-16472; Giurisato, E., et al., (2007) Mol Cell Biol 27(24): 8583-8599; and Wu, J., et al., (2000) J Exp Med 192(7): 1059-1068, the contents of each of which are incorporated herein in their entirety.

The term “anti-tumor effect” as used herein, refers to a biological effect which can be manifested by a decrease in tumor volume, a decrease in the number of tumor cells, a decrease in the number of metastases, an increase in life expectancy, or amelioration of various physiological symptoms associated with the cancerous condition. An “anti-tumor effect” can also be manifested by the ability of the peptides, polynucleotides, cells and antibodies of the invention in prevention of the occurrence of tumor in the first place.

As used herein, the term “autologous” is meant to refer to any material derived from an individual which is later to be re-introduced into the same individual.

As used herein, the term “allogeneic” refers to material derived from an animal which is later introduced into a different animal of the same species.

To “treat” a disease as the term is used herein, means to reduce the frequency or severity of at least one sign or symptom of a disease or disorder experienced by a subject. In one example, a therapy (e.g., administration of a therapeutic agent of the present disclosure) treats a disease or condition by decreasing one or more signs or symptoms associated with the disease or condition, for example as compared to the response in the absence of the therapy. For example, administration of a therapeutic agent may provide an anti-tumor effect that decreases one or more signs or symptoms associated with cancer.

As used herein, the term “administration” means to provide or give a subject one or more agents, such as an agent that treats one or more signs or symptoms associated with a condition/disorder or disease including but not limited to cancer (e.g., lymphoma), viral infection, bacterial infection, etc., by any effective route. Exemplary routes of administration include, but are not limited to, injection (such as subcutaneous, intramuscular, intradermal, intraperitoneal, and intravenous), oral, sublingual, rectal, transdermal, intranasal, vaginal and inhalation routes. Administration “in combination with” one or more further therapeutic agents includes simultaneous (concurrent) and sequential administration in any order.

The term “pharmaceutically acceptable”, as used herein, refers to a material, including but not limited, to a salt, carrier or diluent, which does not abrogate the biological activity or properties of the compound, and is relatively nontoxic, i.e., the material may be administered to an individual without causing undesirable biological effects or interacting in a deleterious manner with any of the components of the composition in which it is contained. The pharmaceutically acceptable carriers (vehicles) useful in this disclosure are conventional. Remington's Pharmaceutical Sciences, by E. W. Martin, Mack Publishing Co., Easton, Pa., 19th Edition (1995), describes compositions and formulations suitable for pharmaceutical delivery of one or more agents, such as one or more modulatory agents. In general, the nature of the carrier will depend on the particular mode of administration being employed. For instance, parenteral formulations can include injectable fluids that include pharmaceutically and physiologically acceptable fluids such as water, physiological saline, balanced salt solutions, aqueous dextrose, glycerol or the like as a vehicle. In addition to biologically-neutral carriers, pharmaceutical agents to be administered can contain minor amounts of non-toxic auxiliary substances, such as wetting or emulsifying agents, preservatives, and pH buffering agents and the like, for example sodium acetate or sorbitan monolaurate, sodium lactate, potassium chloride, calcium chloride, and triethanolamine oleate. For example, the present invention provides a pharmaceutical composition comprising a pharmaceutically acceptable excipient and an, e.g., γδ, T cell, preferably a γδ T cell engineered to express a CAR directed to CD70, as described herein.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

“Isolated” means altered or removed from the natural state. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell.

Unless otherwise specified, a “nucleotide sequence encoding an amino acid sequence” includes all nucleotide sequences that are degenerate versions of each other and that encode the same amino acid sequence. Nucleotide sequences that encode proteins and RNA may include introns.

The terms “patient,” “subject,” “individual,” and the like are used interchangeably herein, and refer to any animal, amenable to the methods described herein. In certain non-limiting embodiments, the patient, subject or individual is a human.

“Expression cassette” refers to a nucleic acid comprising expression control sequences operatively linked to a nucleic acid encoding a transcript or polypeptide to be expressed. An expression cassette comprises sufficient cis-acting elements for expression; other elements for expression can be supplied by the host cell or in an in vitro expression system. Expression cassettes can be a component of a vector such as a cosmid, a plasmid (e.g., naked or contained in a liposome), or a virus (e.g., lentivirus, retrovirus, adenovirus, and adeno-associated virus). An expression cassette can be in a host cell, such as a γδ T cell.

### II. Compositions and Methods of the Invention

Unless defined otherwise, technical and scientific terms used herein have the same meaning as commonly understood by a person of ordinary skill in the art. In particular, this disclosure utilizes routine techniques in the field of recombinant genetics, immunology, and biochemistry. Basic texts disclosing the general terms in molecular biology and genetics include e.g., Lackie, Dictionary of Cell and Molecular Biology, Elsevier (5th ed. 2013). Basic texts disclosing methods in recombinant genetics and molecular biology include e.g., Sambrook et al, Molecular Cloning—A Laboratory Manual, Cold Spring Harbor Press 4th Edition (Cold Spring Harbor, N.Y. 2012) and Current Protocols in Molecular Biology Volumes 1-3, John Wiley & Sons, Inc. (1994-1998) and Supplements 1-115 (1987-2016). Basic texts disclosing the general methods and terms in biochemistry include e.g., Lehninger Principles of Biochemistry sixth edition, David L. Nelson and Michael M. Cox eds. W. H. Freeman (2012). Basic texts disclosing the general methods and terms immunology include Janeway's Immunobiology (Ninth Edition) by Kenneth M. Murphy and Casey Weaver (2017) Garland Science; Fundamental Immunology (Seventh Edition) by William E. Paul (2013) Lippincott, Williams and Wilkins.

### A. Chimeric Antigen Receptor Constructs:

Aspects of the invention include nucleic acids encoding CARs, and constructs and vectors containing such nucleic acids. In some cases, the nucleic acid is a, e.g., heterologous, component of an expression cassette. In some embodiments, the nucleic acid is a, e.g., heterologous, component of a retroviral vector. In some embodiments, the nucleic acid is a, e.g., heterologous, component of an αβ or γδ T cell, and preferably a γδ T cell. In some embodiments, the nucleic acid is a, e.g., heterologous, component of an γ⁺ T cell and/or a δ⁺ T cell. In some embodiments, the nucleic acid is a, e.g., heterologous, component of an α⁻ T cell and/or a β⁻ T cell.

Described herein are nucleic acids encoding a CAR binding domain that specifically binds to a tumor associated antigen (TAA) expressed on a surface of a tumor cell. In embodiments, the TAA is CD70. CD70 belongs to the TNF superfamily and has the amino acid sequence of SEQ ID NO: 1. CD70 is involved in the proliferation and survival of lymphoid-derived cells when it interacts with its receptor, CD27.

An embodiment of the invention provides a nucleic acid encoding a CAR comprising an antigen-binding transmembrane domain comprising a CD27 amino acid sequence. In this regard, the CAR may comprise both a CD27 antigen binding domain and a CD27 transmembrane domain. The CD27 amino acid sequence may comprise or consist of any suitable human antigen binding and transmembrane domain CD27 amino acid sequence. In an embodiment of the invention, full-length CD27, including a signal peptide, antigen binding domain, transmembrane domain, and costimulatory signaling domain, has the amino acid sequence of SEQ ID NO: 33. In an embodiment of the invention, the antigen binding domain of CD27 is composed of amino acid residues 20-191 of SEQ ID NO: 33, and has the amino acid sequence of SEQ ID NO: 35, the transmembrane domain of CD27 is composed of amino acid residues 192-212 of SEQ ID NO: 33, and has the amino acid sequence of SEQ ID NO: 37, and the costimulatory domain of CD27 is composed of amino acid residues 213-260 and has the amino acid sequence of SEQ ID NO: 39. Accordingly, in an embodiment of the invention, the CAR comprises an antigen binding-transmembrane domain comprising the amino acid sequences of SEQ ID NOs: 35 and 37. The antigen binding domain of CD27 specifically binds to CD70.

In an embodiment of the invention, the antigen binding domain of CD27 has the amino acid sequence of SEQ ID NO: 41 (R58W), the transmembrane domain of CD27 has the amino acid sequence of SEQ ID NO: 37, and the intracellular signaling domain of CD27 has the amino acid sequence of SEQ ID NO: 39. Accordingly, in an embodiment of the invention, the CAR comprises an antigen binding-transmembrane domain comprising the amino acid sequences of SEQ ID NOs: 41 and 37.

An embodiment of the invention provides a nucleic acid encoding a CAR comprising an antigen binding-transmembrane domain comprising a CD27 amino acid sequence lacking all or a portion of the CD27 costimulatory domain, wherein the portion that is lacking from the CAR is at least contiguous amino acid residues 238-260, at least contiguous amino acid residues 237-260, at least contiguous amino acid residues 236-260, at least contiguous amino acid residues 235-260, at least contiguous amino acid residues 234-260, at least contiguous amino acid residues 233-260, at least contiguous amino acid residues 232-260, at least contiguous amino acid residues 231-260, at least contiguous amino acid residues 230-260, at least contiguous amino acid residues 229-260, at least contiguous amino acid residues 228-260, at least contiguous amino acid residues 227-260, at least contiguous amino acid residues 226-260, at least contiguous amino acid residues 225-260, at least contiguous amino acid residues 224-260, at least contiguous amino acid residues 223-260, at least contiguous amino acid residues 222-260, at least contiguous amino acid residues 221-260, at least contiguous amino acid residues 220-260, at least contiguous amino acid residues 219-260, at least contiguous amino acid residues 218-260, at least contiguous amino acid residues 217-260, at least contiguous amino acid residues 216-260, at least contiguous amino acid residues 215-260, at least contiguous amino acid residues 214-260, at least contiguous amino acid residues 213-260, or at least contiguous amino acid residues 212-260, of SEQ ID NO: 33.

In an embodiment, the antigen binding-transmembrane domain comprises a CD27 amino acid sequence lacking contiguous amino acid residues 238-260, contiguous amino acid residues 237-260, contiguous amino acid residues 236-260, contiguous amino acid residues 235-260, contiguous amino acid residues 234-260, contiguous amino acid residues 233-260, contiguous amino acid residues 232-260, contiguous amino acid residues 231-260, contiguous amino acid residues 230-260, contiguous amino acid residues 229-260, contiguous amino acid residues 228-260, contiguous amino acid residues 227-260, contiguous amino acid residues 226-260, contiguous amino acid residues 225-260, contiguous amino acid residues 224-260, contiguous amino acid residues 223-260, contiguous amino acid residues 222-260, contiguous amino acid residues 221-260, contiguous amino acid residues 220-260, contiguous amino acid residues 219-260, contiguous amino acid residues 218-260, contiguous amino acid residues 217-260, contiguous amino acid residues 216-260, contiguous amino acid residues 215-260, contiguous amino acid residues 214-260, contiguous amino acid residues 213-260, or contiguous amino acid residues 212-260, of SEQ ID NO: 33.

A CD27 amino acid sequence lacking all or a portion of the CD27 costimulatory domain is also referred to herein as a “truncated CD27 amino acid sequence” or a “truncated CD27.” Accordingly, in embodiments a nucleic acid as disclosed herein encodes a truncated CD27 amino acid sequence.

In a preferred embodiment, a nucleic acid encodes a CAR comprising a CD27 amino acid sequence composed of at least an antigen binding domain comprising or consisting of SEQ ID NO: 35 or SEQ ID NO: 41, a transmembrane domain comprising or consisting of SEQ ID NO: 37, and a costimulatory domain comprising or consisting of SEQ ID NO: 39, and optionally further comprises a signal peptide, preferably where said signal peptide comprises or consists of SEQ ID NO: 2.

In embodiments, a CAR encoded by a nucleic acid may further comprise at least one costimulatory domain, wherein the costimulatory domain comprises functional costimulatory signaling domain derived from e.g., a MHC class I molecule, TNF receptor proteins, immunoglobulin-like proteins, cytokine receptors, integrins, signaling lymphocytic activation molecules (SLAM proteins), activating NK cell receptors, BTLA, a Toll ligand receptor, and the like. For example, it is within the scope of this disclosure that the CAR can include 2, 3, 4 or more costimulatory domains. It is also within the scope of this disclosure that when more than one costimulatory domain is included, the costimulatory domains may be the same, or they may be different. In embodiments, the costimulatory domains are derived from one or more of TLR1, TLR2, TLR3, TLR4, TLR5, TLR6, TLR7, TLR8, TLR9, TLR10, CARD11, B7-H3, CEACAM1, CRTAM, CD2, CD3C, CD4, CD7, CD8α, CD8β, CD11a, CD11b, CD11c, CD11d, IL2Rβ, IL2γ, IL7Rα, IL4R, IL7R, IL15R, IL21R, CD18, CD19, CD19a, CD27, CD28, CD29, CD30, CD40, CDS, CD49a, CD49D, CD49f, CD54 (ICAM), CD69, CD70, CD80, CD83, CD84, CD86, CD96 (Tactile), CD100 (SEMA4D), CD103, CD134 (OX40), CD137 (4-1BB), CD152 (CTLA-4), CD160 (BY55), CD162 (SELPLG), CD244 (2B4), CD270 (HVEM), CD226 (DNAM1), CD229 (Ly9), CD278 (ICOS), ICAM-1, LFA-1 (CD11a/CD18), FcR, FcγRI, FcγRII, FcγRIII, LAT, NKG2C, SLP76, TRIM, ZAP70, GITR, BAFFR, LTBR, LAT, GADS, LIGHT, HVEM (LIGHTR), KIRDS2, ITGA4, ITGA6, ITGAD, ITGAE, ITGAL, ITGAM, ITGAX, ITGB1, ITGB2, ITGB7, NKG2C, NKG2D, IA4, VLA-1, VLA-6, SLAM (SLAMF1, CD150, IPO-3), SLAMF4, SLAMF6 (NTB-A, Ly108), SLAMF7, SLAMF8 (BLAME), SLP-76, PAG/Cbp, NKp80 (KLRF1), NKp44, NKp30, NKp46, BTLA, JAML, CD150, PSGL1, TSLP, TNFR2, and TRANCE/RANKL, or a portion thereof, and combinations thereof.

In some embodiments, a nucleic acid encoding a CAR encodes at least one 4-1BB costimulatory domain, and optionally a second costimulatory domain selected from 4-1BB, 2B4, ICOS, CD28, OX40, and CD27 costimulatory domains, or any of the above-mentioned costimulatory domains. In some embodiments, the nucleic acid encodes at least two 4-1BB costimulatory domains, or at least two 4-1BB costimulatory domains in combination with one, two, three, or four, or more, costimulatory domains selected from 4-1BB, ICOS, CD28, OX40, and CD27, or any of the above-mentioned costimulatory domains. In some embodiments, the 4-1BB costimulatory domain comprises SEQ ID NO: 4. In some embodiments, the 4-1BB costimulatory domain comprises an amino acid sequence having at least one, at least two, or at least three or more modifications of an amino acid sequence of SEQ ID NO: 4. In embodiments, the 4-1BB costimulatory domain is substantially similar to the 4-1BB costimulatory domain comprising SEQ ID NO: 4.

In some embodiments, a nucleic acid encoding a CAR encodes at least one CD27 costimulatory domain, and optionally at least one second costimulatory domain selected from 4-1BB, ICOS, CD28, OX40, 2B4, and CD27 costimulatory domains, or any of the above-mentioned costimulatory domains. In some embodiments, the nucleic acid encodes at least one CD27 costimulatory domain, and a 4-IBB costimulatory domain. In some embodiments, the nucleic acid encodes two CD27 costimulatory domains, and at least one second costimulatory domain selected from a 4-1BB, ICOS, CD28, and CD27. In some embodiments, the CD27 costimulation domain comprises SEQ ID NO: 39. In some embodiments, the CD27 costimulatory domain comprises an amino acid sequence having at least one, at least two, at least three or more modifications of an amino acid sequence of SEQ ID NO: 39. In embodiments, the CD27 costimulatory domain is substantially similar to the CD27 costimulatory domain comprising SEQ ID NO: 39.

In some embodiments, a nucleic acid encoding a CAR encodes at least one CD28 costimulatory domain, and optionally a second costimulatory domain selected from 4-1BB, 2B4, ICOS, CD28, OX40, and CD27 costimulatory domains, or any of the above-mentioned costimulatory domains. In some embodiments, the nucleic acid encodes at least two CD28 costimulatory domains, or at least two CD28 costimulatory domains in combination with one, two, three, or four, or more, costimulatory domains selected from a 4-1BB, ICOS, CD28, OX40, and CD27, or any of the above-mentioned costimulatory domains. In some embodiments, the CD28 costimulatory domain comprises SEQ ID NO: 73. In embodiments, the CD28 costimulatory domain comprises SEQ ID NO: 74. Included in SEQ ID NO: 73 and SEQ ID NO: 74 are three subdomains YMNM, PRRP, and PYAP, that are capable to regulate signaling pathways. In embodiments, a disclosed CAR comprises mutation or deletion of one or more of said subdomains (see e.g., WO2019010383). In some embodiments, the CD28 costimulatory domain comprises an amino acid sequence having at least one, at least two, at least three or more modifications of an amino acid sequence of SEQ ID NO: 73, or an amino acid sequence of SEQ ID NO: 74. In some embodiments, the CD28 costimulatory domain is substantially similar to the CD28 costimulatory domain comprising SEQ ID NO: 73. In some embodiments, the CD28 costimulatory domain is substantially similar to the CD28 costimulatory domain comprising SEQ ID NO: 74.

In some embodiments, a nucleic acid encoding a CAR encodes at least one ICOS costimulatory domain, and optionally a second costimulatory domain selected from 4-1BB, 2B4, ICOS, CD28, OX40, and CD27 costimulatory domains, or any of the above-mentioned costimulatory domains. In some embodiments, the nucleic acid encodes at least two ICOS costimulatory domains, or at least two ICOS costimulatory domains in combination with one, two, three, or four, or more, costimulatory domains selected from 4-1BB, ICOS, CD28, OX40, and CD27, or any of the above-mentioned costimulatory domains. In some embodiments, the ICOS costimulatory domain comprises SEQ ID NO: 75. In some embodiments, the ICOS costimulatory domain comprises an amino acid sequence having at least one, at least two, at least three or more modifications of an amino acid sequence of SEQ ID NO: 75 (see e.g., US20170209492). In some embodiments, the ICOS costimulatory domain is substantially similar to the ICOS costimulatory domain comprising SEQ ID NO: 75.

In some embodiments, a nucleic acid encoding a CAR encodes at least one OX40 costimulatory domain, and optionally a second costimulatory domain selected from 4-1BB, 2B4, ICOS, CD28, OX40, and CD27 costimulatory domains, or any of the above-mentioned costimulatory domains. In some embodiments, the nucleic acid encodes at least two OX40 costimulatory domains, or at least two OX40 costimulatory domains in combination with one, two, three, or four, or more, costimulatory domains selected from 4-1BB, ICOS, CD28, OX40, and CD27, or any of the above-mentioned costimulatory domains. In some embodiments, the OX40 costimulatory domain comprises SEQ ID NO: 76. In some embodiments, the OX40 costimulatory domain comprises an amino acid sequence having at least one, at least two, at least three or more modifications of an amino acid sequence of SEQ ID NO: 76. In some embodiments, the OX40 costimulatory domain is substantially similar to the OX40 costimulatory domain comprising SEQ ID NO: 76.

In embodiments, a nucleic acid encoding a CAR encodes at least one intracellular signaling domain. In embodiments, the at least one intracellular signaling domain is additional to one or more costimulatory domains. In embodiments, the one or more intracellular signaling domains are included to increase proliferation, persistence, and/or cytotoxic activity of the host cell, preferably a γδ cell, harboring the CAR as herein disclosed. For example, in some embodiments, the intracellular signaling domain(s) comprise CD3ζ, repeat (e.g., 2-5) DAP10 YINM motifs, signaling domains derived from LFA-1, DAP12, FcRγ, FcRβ, CD3γ, CD3δ, CD3ε, CD79a, CD79b, CD5, CD22, FcεRI, CD66d, and the like. It is within the scope of this disclosure that the endodomain of a disclosed CAR can include a plurality (e.g., 2, 3, 4, or more) of intracellular signaling domains. In a case where more than one intracellular signaling domain is included, the intracellular signaling domains may be the same, or they may be different.

In some embodiments, an intracellular signaling domain of a disclosed CAR is or comprises a CD3ζ signaling domain. In some embodiments, a CD3ζ signaling domain is or comprises the amino acid sequence set forth in SEQ ID NO: 10, 12, or 13.

In some embodiments a nucleic acid encoding a CAR encodes one or more costimulatory domain(s) (e.g., 4-1BB costimulatory domain), and one or more intracellular signaling domain(s) (e.g., CD3ζ signaling domain). In some embodiments, the nucleic acid encodes at least one 4-1BB costimulatory domain, at least one CD27 or CD28 domain, and at least one CD3ζ signaling domain. In other embodiments, the nucleic acid encodes one or more first costimulatory domains (e.g., 4-1BB, CD27, CD28, OX40, ICOS) and one or more second costimulatory domains (e.g., 4-1BB, CD27, CD28, OX40, ICOS), and one or more intracellular signaling domains (e.g., CD3ζ). In embodiments, the CD3ζ signaling domain is downstream (C-terminal) to the costimulatory domain(s) (e.g., 4-1BB). In some embodiments, the CD3v signaling domain is upstream (N-terminal) to the costimulatory domain(s) (e.g., 4-1BB).

In embodiments, an isolated nucleic acid encoding a CAR of the subject invention can also encode for one or more multicistronic linker region(s) configured to facilitate translation of the CAR polypeptide and one or more additional polypeptides. In embodiments, nucleic acids encoding the one or more additional polypeptides and associated linker region can be positioned at the 3′ end of the isolated nucleic acid, or at the 5′ end of the isolated nucleic acid, or in some examples at both the 5′ end and the 3′ end of the isolated nucleic acid. In some examples, the linker region(s) can encode a self-cleavage and/or a cleavage polypeptide sequence. In some examples, the self-cleavage sequence is a 2A self-cleaving sequence (e.g., T2A, P2A, E2A, F2A) which can induce ribosomal skipping during translation of the CAR. In embodiments, the cleavage sequence is a furin sequence. In some examples, the cleavage sequence (e.g., furin cleavage sequence as set forth in SEQ ID NO: 79) is amino terminal to a self-cleavage sequence, for example furin-P2A (FP2A). In some embodiments, the multicistronic linker region encodes an internal ribosome entry site. In some embodiments, the addition of an optional linker “GSG” or “SGSG” and the like can improve cleavage efficiency. In this way, the one or more additional polypeptides can be release from the CAR and directed to the secretory pathway.

In some embodiments, the cleavage sequence is the FP2A amino acid sequence as set forth in SEQ ID NO: 6. In some embodiments, the cleavage sequence is a P2A amino acid sequence as set forth in SEQ ID NO: 8, or SEQ ID NOs: 77-78. In some embodiments, the cleavage sequence is a furin amino acid sequence as set forth in SEQ ID NO: 79. In some embodiments, the cleavage sequence is a F2A amino acid sequence as set forth in SEQ ID NO: 80. In some embodiments, the cleavage sequence is a E2A amino acid sequence as set forth in SEQ ID NO: 81. In some embodiments, the cleavage sequence is a T2A amino acid sequence as set forth in SEQ ID NO: 82. In certain aspects, multiple cleavage and/or self-cleavage sequences can be encoded carboxy-terminal to signaling and/or costimulatory domain(s) and amino-terminal to an encoded one or more additional polypeptide. In certain aspects, one or more self-cleavage sequences and one or more sequences cleaved by an endogenous protease are encoded in a construct described herein. In certain embodiments, an endogenous protease recognition site is encoded amino terminal to a self-cleavage sequence. Further suitable internal ribosome entry sites include, but are not limited to, those disclosed e.g., in Nucleic Acids Res. 2010 January; 38 (Database issue): D131-6. doi: 10.1093/nar/gkp981. Epub 2009 Nov. 16, those described at iresite.org, those described in WO 2018/215787, the sequence described in GenBank accession No. KP019382.1, and the IRES element disclosed in GenBank accession No. LT727339.1. Additional multi-cistronic linker regions, including cleavage self-cleavage, and IRES elements, are disclosed in US 2018/0360992 and U.S. Pat. No. 8,865,467

In some embodiments, the multi-cistronic linker region encodes an internal ribosome entry site. An exemplary internal ribosome entry site is encoded by the nucleotide sequence set forth in SEQ ID NO: 83. Another exemplary internal ribosome entry site is encoded by the nucleotide sequence set forth in SEQ ID NO: 84.

In embodiments, the one or more additional polypeptides include one or more soluble gamma chain cytokines expressed as separate polypeptides from the CAR. The one or more soluble common gamma chain cytokines can include but are not limited to IL-2, IL-4, IL-7, IL-9, IL-15, IL-21, IL-23. In some embodiments, the common gamma chain cytokine is selected from IL-2, IL-7, and IL-15. In some embodiments, the common gamma chain cytokine is IL-15. IL-15 sequences, including codon optimized nucleic acid sequences encoding soluble IL-15 (sIL-15) are disclosed herein and in WO 2007/037780.

In embodiments, the one or more additional polypeptides include one or more labels or markers, for example to facilitate an ability to monitor CAR expression level, serve as an internal control, and the like. In some embodiments, an isolated nucleic acid encoding a CAR encodes for a fluorescent protein, examples of which include but are not limited to green fluorescent protein (GFP), red fluorescent protein (RFP), enhanced GFP (EGFP), enhanced cyan fluorescent protein (ECFP), enhanced yellow fluorescent protein (EYFP), and the like. Other examples can include but are not limited to chloramphenicol acetyltransferase, beta-galactosidase, beta-glucuronidase, beta-lactamase, luciferase, and the like.

In embodiments, the one or more additional polypeptides include a protein that is expressed on a cell surface to facilitate detection and/or isolation of cells expressing said protein, e.g., via fluorescent activated cell sorting (FACS); or for enrichment through positive selection using an antibody specific to the encoded protein, e.g., use of an antibody to purify or enrich the cells product on a column or apparatus; or for in vivo binding of an antibody to the protein to enhance or eliminate activity, e.g., to facilitate removal of cells expressing the protein in patients as a safety consideration. Exemplary proteins useful for these purposes include, e.g., CD19, CD20 (Rituximab recognition domain), LNGFR, a truncated form of the human epidermal growth factor receptor (EGFRt), and the like. By way of example, EGFRt can be targeted by a clinical stage antibody, where such treatment of a patient with said antibody results in elimination of cells containing an isolated nucleic acid encoding a CAR and/or said CAR as disclosed herein.

In embodiments, the one or more additional polypeptides include a protein that functions to increase resistance to exhaustion and activation-induced apoptosis and/or upregulate one or more proinflammatory cytokines, costimulatory molecules and/or antigen presentation machinery. A representative example includes but is not limited to lymphotoxin beta receptor (LTBR). LTBR is typically expressed in a subset of myeloid cells but is absent in lymphocytes. When expressed in T cells, LTBR may induce transcriptional remodeling that imparts the T cell with one or more of the above-mentioned advantageous functions (Legut et al., Blood. (2021); 138(1): 1726).

In some embodiments, the one or more additional polypeptides includes a polypeptide that imparts host cells with the capability to resist tumor antigen-specific cellular immunity, for example that mediated by transforming growth factor beta (TGF-β). For example, an isolated nucleic acid may encode a dominant negative receptor for TGF-beta (dnTGFβR2), e.g., as described in Foster et al., J Immunother. (2008); 31:500-505, WO2019/173324A1, WO2020/183131A1, and WO2020042647A1. Incorporation of such a dominant negative receptor for TGF-beta may provide a functional advantage over control cells that lack such a dominant negative receptor for TGF-beta in the presence of a TGF-beta-secreting tumor, including enhanced anti-tumor activity. In some embodiments, an isolated nucleic acid encodes a signal peptide operably linked to facilitate directing of the one or more additional polypeptides to the secretory pathway. Such one or more additional polypeptides can be those that reside inside certain organelles, are secreted from the host cell, or are inserted into cellular membranes. In embodiments, the signal peptide comprises or consists of the amino acid sequence set forth as SEQ ID NO: 2. In embodiments, the signal peptide comprises or consists of the amino acid sequence set forth as SEQ ID NO: 17. In embodiments, the signal peptide comprises or consists of the amino acid sequence set forth as SEQ ID NO: 21. In embodiments, the signal peptide comprises or consists of the amino acid sequence set forth as SEQ ID NO: 25. In embodiments, the signal peptide comprises or consists of the amino acid sequence set forth as SEQ ID NO: 29. In embodiments, the signal peptide comprises or consists of the amino acid sequence set forth as SEQ ID NO: 85

In some embodiments, the one or more additional polypeptides includes the EGFRt amino acid sequence as set forth in SEQ ID NO: 19. In embodiments, the signal peptide comprising or consisting of the amino acid sequence set forth as SEQ ID NO: 17 is operably linked to SEQ ID NO: 19. In some embodiments, the one or more additional polypeptides includes the dominant-negative TGFβ receptor II (dnTGFβR2) amino acid sequence as set forth in SEQ ID NO: 23. In embodiments, the signal peptide comprising or consisting of the amino acid sequence set forth as SEQ ID NO: 21 is operably linked to SEQ ID NO: 23. In some embodiments, the one or more additional polypeptides includes the full-length LTBR amino acid sequence as set forth in SEQ ID NO: 27. In embodiments, the signal peptide comprising or consisting of the amino acid sequence set forth as SEQ ID NO: 25 is operably linked to SEQ ID NO: 27. In some embodiments, the one or more additional polypeptides includes the LNGFR amino acid sequence as set forth in SEQ ID NO: 31. In embodiments, the signal peptide comprising or consisting of the amino acid sequence set forth as SEQ ID NO: 29 is operably linked to SEQ ID NO: 31. In some embodiments, the one or more additional polypeptides includes the sIL-15 amino acid sequence as set forth in SEQ ID NO: 86. In embodiments, the signal peptide comprising or consisting of the amino acid sequence as set forth in SEQ ID NO: 85 is operably linked to SEQ ID NO: 86.

In some embodiments, the one or more additional polypeptides includes a chimeric switch receptor comprising an extracellular domain of a TGFβ receptor for binding to TGFβ (e.g., TGFβRI and/or TGFβRII), and an intracellular domain of a cytokine receptor. The chimeric switch receptors can convert a TGFβ signal into a cytokine signal that promotes cytotoxicity. Examples of such chimeric switch receptors include those descried in WO2012138858, WO2016122738, WO2018094244, WO2014172584, WO2019109980, and WO2022037562, each of which is incorporated by reference in its entirety.

In embodiments, the one or more additional polypeptides includes a dominant negative Fas (dnFas). Incorporation of such a dominant negative Fas in a T cell may provide a functional advantage over control cells that lack such a dominant negative Fas in the prevention of Fas ligand-induced apoptosis and allowing for T cell persistence and antitumor efficacy. Examples of the dnFas include that described in Yamamoto T N et al., T cells genetically engineered to overcome death signaling enhance adoptive cancer immunotherapy, J Clin Invest. 2019 Feb. 25; 129(4):1551-1565, which is incorporated by reference herein in its entirety.

In embodiments, the one or more additional polypeptides includes a membrane-bound IL-12 (mbIL-12). Incorporation of such a mbIL-12 in a T cell may provide a functional advantage over control cells that lack such a mbIL-12 in enhancing effector functions of the T cells and/or limiting the systemic toxicity associated with IL-12. Examples of the mbIL-12 include those described in Hu J. et al., Cell membrane-anchored and tumor-targeted IL-12 (attIL12)-T cell therapy for eliminating large and heterogeneous solid tumors, J Immunother Cancer. 2022 January; 10(1):e003633; Hombach A. et al., IL12 integrated into the CAR exodomain converts CD8+ T cells to poly-functional NK-like cells with superior killing of antigen-loss tumors, Mol Ther. 2022 Feb. 2; 30(2):593-605; and Lee E H et al., Antigen-dependent IL-12 signaling in CAR T cells promotes regional to systemic disease targeting, bioRxiv. 2023 Jan. 7; 2023.01.06.522784, each of which is incorporated by reference herein in its entirety.

The present disclosure provides CARs with “substantial identity” or “substantial similarity” to the sequences provided herein. The term “substantial identity” or “substantially identical,” when referring to a nucleic acid or fragment thereof, indicates that, when optimally aligned with another nucleic acid (or the complementary strand of the other nucleic acid), there is nucleotide sequence identity in %, for example, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least 99.5%, or 100% of the nucleotide bases, as measured by any well-known algorithm of sequence identity, such as FASTA, BLAST or GAP, as discussed below. A nucleic acid molecule having substantial identity to a reference nucleic acid molecule may, in certain instances, encode a polypeptide having the same or substantially similar amino acid sequence as the polypeptide encoded by the reference nucleic acid molecule.

As applied to polypeptides, the term “substantial similarity” or “substantially similar” means that two peptide sequences, when optimally aligned, such as by the programs GAP or BESTFIT using default gap weights, share at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least 99.5%, or 100% sequence identity. In some aspects, residue positions, which are not identical, differ by conservative amino acid substitutions. A “conservative amino acid substitution” is one in which an amino acid residue is substituted by another amino acid residue having a side chain (R group) with similar chemical properties (e.g., charge or hydrophobicity). In general, a conservative amino acid substitution will not substantially change the functional properties of a protein. In cases where two or more amino acid sequences differ from each other by conservative substitutions, the percent or degree of similarity may be adjusted upwards to correct for the conservative nature of the substitution. Means for making this adjustment are well known to those of skill in the art. See, e.g., Pearson (1994) Methods Mol. Biol. 24:307-331, which is herein incorporated by reference. Examples of groups of amino acids that have side chains with similar chemical properties include 1) aliphatic side chains: glycine, alanine, valine, leucine and isoleucine; 2) aliphatic-hydroxyl side chains: serine and threonine; 3) amide-containing side chains: asparagine and glutamine; 4) aromatic side chains: phenylalanine, tyrosine, and tryptophan; 5) basic side chains: lysine, arginine, and histidine; 6) acidic side chains: aspartate and glutamate, and 7) sulfur-containing side chains: cysteine and methionine. Preferred conservative amino acids substitution groups are: valine-leucine-isoleucine, phenylalanine-tyrosine, lysine-arginine, alanine-valine, glutamate-aspartate, and asparagine-glutamine. Alternatively, a conservative replacement is any change having a positive value in the PAM250 log-likelihood matrix disclosed in Gonnet et al. (1992) Science 256:1443 45, herein incorporated by reference. A “moderately conservative” replacement is any change having a nonnegative value in the PAM250 log-likelihood matrix.

Sequence identity and/or similarity for polypeptides is typically measured using sequence analysis software. Protein analysis software matches similar sequences using measures of similarity assigned to various substitutions, deletions and other modifications, including conservative amino acid substitutions. For instance, GCG software contains programs such as GAP and BESTFIT which can be used with default parameters to determine sequence homology or sequence identity between closely related polypeptides, such as homologous polypeptides from different species of organisms or between a wild type protein and a mutein thereof. See, e.g., GCG Version 6.1. Polypeptide sequences also can be compared using FASTA with default or recommended parameters; a program in GCG Version 6.1. FASTA (e.g., FASTA2 and FASTA3) provides alignments and percent sequence identity of the regions of the best overlap between the query and search sequences (Pearson (2000) supra). Sequences also can be compared using the Smith-Waterman homology search algorithm using an affine gap search with a gap open penalty of 12 and a gap extension penalty of 2, BLOSUM matrix of 62. Another preferred algorithm when comparing a sequence disclosed herein to a database containing a large number of sequences from different organisms is the computer program BLAST, especially BLASTP or TBLASTN, using default parameters. See, e.g., Altschul et al. (1990) J. Mol. Biol. 215:403-410 and (1997) Nucleic Acids Res. 25:3389-3402, each of which is herein incorporated by reference.

Alternatively or additionally, a parent CAR of the present disclosure can comprise at least one non-conservative amino acid substitution. In this case, it is preferable for the non-conservative amino acid substitution to not interfere with or inhibit the biological activity of the parent CAR. The non-conservative amino acid substitution may enhance the biological activity of the modified CAR, such that the biological activity of the modified CAR is increased as compared to the parent CAR.

In some embodiments, an isolated nucleic acid encodes SEQ ID NO: 45, a CAR polypeptide PL648 comprising the following domains in order: a full-length CD27 domain, a 4-1BB costimulatory domain, a CD3ζ signaling domain, an FP2A cleavage domain, and an EGFP domain.

In some embodiments, an isolated nucleic acid encodes SEQ ID NO: 47, a CAR polypeptide PL1090 comprising the following domains in order: a full-length CD27 domain, a 4-1BB costimulatory domain, a CD3ζ signaling domain, an FP2A cleavage domain, a signal peptide, and an EGFRt domain.

In some embodiments, an isolated nucleic acid encodes SEQ ID NO: 49, a CAR polypeptide PL1093 comprising the following domains in order: a full-length CD27 domain, a 4-1BB costimulatory domain, a CD3ζ signaling domain, an FP2A cleavage domain, a signal peptide, and a dnTGFβR2 domain.

In some embodiments, an isolated nucleic acid encodes SEQ ID NO: 51, a CAR polypeptide PL1106 comprising the following domains in order: a full-length CD27 domain, a 4-1BB costimulatory domain, a CD3ζ signaling domain, an FP2A cleavage domain, a signal peptide, and a LTBR domain.

In some embodiments, an isolated nucleic acid encodes SEQ ID NO: 53, a CAR polypeptide PL1107 comprising the following domains in order: a full-length CD27 domain, a 4-1BB costimulatory domain, and a CD3ζ signaling domain.

In some embodiments, an isolated nucleic acid encodes SEQ ID NO: 55, a CAR polypeptide PL1110 comprising the following domains in order: a full-length CD27 domain, a 4-1BB costimulatory domain, a CD3ζ signaling domain, an FP2A signaling domain, and an LNGFRt domain.

In some embodiments, an isolated nucleic acid encodes SEQ ID NO: 57, a CAR polypeptide PL1179 comprising the following domains in order: a full-length CD27 domain, a 4-1BB costimulatory domain, a CD3ζ signaling domain, a P2A cleavage domain, a signal peptide, and a dnTGFβR2 domain.

In some embodiments, an isolated nucleic acid encodes SEQ ID NO: 59, a CAR polypeptide PL1099 comprising the following domains in order: a full-length CD27 domain, a 4-1BB costimulatory domain, a CD3ζ signaling domain, a P2A cleavage domain, a signal peptide, and a LNGFRt domain.

In any of the above-mentioned CAR polypeptides encoded by said isolated nucleic acids, a FP2A cleavage domain may be substituted by a P2A cleavage domain (or other cleavage domain), and vice versa. Additionally or alternatively, any of the above-mentioned CAR polypeptides may include an R58W modification.

In some embodiments, a nucleic acid encoding a PL648 CAR comprises the sequence of SEQ ID NO: 46.

In some embodiments, a nucleic acid encoding a PL1090 CAR comprises the sequence of SEQ ID NO: 48.

In some embodiments, a nucleic acid encoding a PL1093 CAR comprises the sequence of SEQ ID NO: 50.

In some embodiments, a nucleic acid encoding a PL1106 CAR comprises the sequence of SEQ ID NO: 52.

In some embodiments, a nucleic acid encoding a PL1107 CAR comprises the sequence of SEQ ID NO: 54.

In some embodiments, a nucleic acid encoding a PL1110 CAR comprises the sequence of SEQ ID NO: 56.

In some embodiments, a nucleic acid encoding a PL1179 CAR comprises the sequence of SEQ ID NO: 58.

In some embodiments, a nucleic acid encoding a PL1099 CAR comprises the sequence of SEQ ID NO: 60.

In some embodiments, an isolated nucleic acid encodes SEQ ID NO: 61, a CAR polypeptide PL1092 comprising the following domains in order: a full-length CD27 domain comprising an R58W modification, a 4-1BB costimulatory domain, a CD3ζ signaling domain, a FP2A cleavage domain, and an EGFRt domain.

In some embodiments, an isolated nucleic acid encodes SEQ ID NO: 63, a CAR polypeptide PL1095 comprising the following domains in order: a full-length CD27 domain comprising an R58W modification, a 4-1BB costimulatory domain, a CD3ζ signaling domain, a FP2A cleavage domain, and an EGFP domain.

In some embodiments, an isolated nucleic acid encodes SEQ ID NO: 65, a CAR polypeptide 1109 comprising the following domains in order: a full-length CD27 domain comprising an R58W modification, a 4-1BB costimulatory domain, and a CD3ζ signaling domain.

In any of the above-mentioned CAR polypeptides comprising an R58W modification, an FP2A cleavage domain may be substituted by a P2A cleavage domain (or other cleavage domain). Additionally or alternatively, another additional polypeptide, for example dnTGFβR2, may be substituted for EGFP, or EGFRt.

In some embodiments, a nucleic acid encoding a PL1092 CAR comprises the sequence of SEQ ID NO: 62.

In some embodiments, a nucleic acid encoding a PL1095 CAR comprises the sequence of SEQ ID NO: 64.

In some embodiments, a nucleic acid encoding a PL1109 CAR comprises the sequence of SEQ ID NO: 66.

In some embodiments, an isolated nucleic acid encodes SEQ ID NO: 67, a CAR polypeptide PL1091 comprising the following domains in order: a truncated CD27 domain, a 4-1BB costimulatory domain, a CD3ζ signaling domain, a FP2A cleavage domain, and an EGFRt domain.

In some embodiments, an isolated nucleic acid encodes SEQ ID NO: 69, a CAR polypeptide PL1094 comprising the following domains in order: a truncated CD27 domain, a 4-1BB costimulatory domain, a CD3ζ signaling domain, a FP2A cleavage domain, and an EGFP domain.

In some embodiments, an isolated nucleic acid encodes SEQ ID NO: 71, a CAR polypeptide PL1108 comprising the following domains in order: a truncated CD27 domain, a 4-1BB costimulatory domain, and a CD3ζ signaling domain.

In any of the above-mentioned CAR polypeptides comprising a truncated CD27, an FP2A cleavage domain may be substituted by a P2A cleavage domain (or other cleavage domain). Additionally or alternatively, another additional polypeptide, for example dnTGFβR2, may be substituted for EGFP, or EGFRt.

In some embodiments, a nucleic acid encoding a PL1091 CAR comprises the sequence of SEQ ID NO: 68.

In some embodiments, a nucleic acid encoding a PL1094 CAR comprises the sequence of SEQ ID NO: 70.

In some embodiments, a nucleic acid encoding a PL1108 CAR comprises the sequence of SEQ ID NO: 72.

Representative CARs of the present disclosure are summarized in Table 1 along with associated SEQ ID NOs corresponding to CAR amino acid sequence. Additional sequences relevant to the present disclosure are summarized in Table 1 below.

TABLE 1
Description of CARs (amino acid)

| | |
|---|---|
| PL648 (SEQ ID NO: 45) | Full-length CD27, 41BB, CD3z - FP2A - EGFP |
| PL1090 (SEQ ID NO: 47) | Full-length CD27, 41BB, CD3z - FP2A - EGFRt |
| PL1093 (SEQ ID NO: 49) | Full-length CD27, 41BB, CD3z - FP2A - dnTGFBR2 |
| PL1106 (SEQ ID NO: 51) | Full-length CD27, 41BB, CD3z - FP2A - LTBR |
| | (Tumor necrosis factor receptor superfamily member 3) |
| PL1107 (SEQ ID NO: 53) | Full-length CD27, 41BB, CD3z |
| PL1110 (SEQ ID NO: 55) | Full-length CD27, 41BB, CD3z-FP2A-LNGFRt |
| PL1179 (SEQ ID NO: 57) | Full-length CD27, 41BB, CD3z - P2A - dnTGFBR2 |
| PL1099 (SEQ ID NO: 59) | Full-length CD27, 4-1BB, CD3z - P2A - LNGFRt |
| PL1092 (SEQ ID NO: 61) | Full-length CD27 with R58W mutation, 41BB, CD3z - FP2A - EGFRt |
| PL1095 (SEQ ID NO: 63) | Full-length CD27 with R58W mutation, 41BB, CD3z - FP2A - EGFP |
| PL1109 (SEQ ID NO: 65) | Full-length CD27 with R58W mutation, 41BB, CD3z |
| PL1091 (SEQ ID NO: 67) | Truncated CD27, 41BB, CD3z - FP2A - EGFRt |
| PL1094 (SEQ ID NO: 69) | Truncated CD27, 41BB, CD3z - FP2A - EGFP |
| PL1108 (SEQ ID NO: 71) | Truncated CD27, 41BB, CD3z |

In some embodiments, an isolated nucleic acid encodes a CAR polypeptide comprising the following domains in order: a protease resistant CD27 comprising a CD8α hinge and transmembrane domain, a 4-1BB costimulatory domain, and a CD3ζ intracellular signaling domain, as described in Leick et al., Cancer Cell (2022); 40:1-15, the contents of which is incorporated by reference herein in its entirety). Other costimulatory and/or intracellular signaling domains as herein disclosed may be additionally or alternatively included in such a CAR polypeptide. Furthermore, other hinge and/or transmembrane domains are within the scope of this disclosure for generation of a CAR polypeptide comprising a protease resistant CD27.

For example and without limitation, a transmembrane domain may be derived either from a natural or from a synthetic source. Where the source is natural, the domain may be derived from any membrane-bound or transmembrane protein. Transmembrane regions of particular use in this invention may be derived from (i.e. comprise at least the transmembrane region(s) of) 4-1BB/CD137, activating NK cell receptors, an Immunoglobulin protein, B7-H3, BAFFR, BLAME (SLAMF8), BTLA, CD28, CD3 epsilon, CD45, CD4, CD5, CD8, CD9, CD16, CD22, CD33, CD37, CD64, CD80, CD86, CD134, CD137, or CD154, CD100 (SEMA4D), CD103, CD160 (BY55), CD18, CD19, CD19a, CD2, CD247, CD27, CD276 (B7-H3), CD28, CD29, CD3 delta, CD3 epsilon, CD3 gamma, CD3 zeta, CD30, CD4, CD40, CD49a, CD49D, CD49f, CD69, CD7, CD84, CD8, CD8alpha, CD8beta, CD96 (Tactile), CD11a, CD11b, CD11c, CD11d, CDS, CEACAM1, CRT AM, cytokine receptor, DAP10, DNAM1 (CD226), Fc gamma receptor, GADS, GITR, HVEM (LIGHTR), IA4, ICAM-1, Ig alpha (CD79a), IL-2R beta, IL-2R gamma, IL-7R alpha, inducible T cell costimulator (ICOS), integrins, ITGA4, ITGA6, ITGAD, ITGAE, ITGAL, ITGAM, ITGAX, ITGB2, ITGB7, ITGB1, KIRDS2, LAT, LFA-1, a ligand that specifically binds with CD83, LIGHT, LTBR, Ly9 (CD229), lymphocyte function-associated antigen-1 (LFA-1; CD11a/CD18), MHC class 1 molecule, NKG2C, NKG2D, NKp30, NKp44, NKp46, NKp80 (KLRF1), OX-40, PAG/Cbp, programmed death-1 (PD-1), PSGL1, SELPLG (CD162), Signaling Lymphocytic Activation Molecules (SLAM proteins), SLAM (SLAMF1; CD150; IPO-3), SLAMF4 (CD244; 2B4), SLAMF6 (NTB-A; Ly108), SLAMF7, SLP-76, TNF receptor proteins, TNFR2, TNFSF14, a Toll ligand receptor, TRANCE/RANKL, VLA1, or VLA-6, or a fragment, truncation, or a combination thereof. Alternatively, the transmembrane domain may be synthetic, in which case it will comprise predominantly hydrophobic residues such as leucine and valine. Preferably a triplet of phenylalanine, tryptophan and valine will be found at each end of a synthetic transmembrane domain.

For example and without limitation, a hinge region relevant to the present disclosure may be derived from the extracellular regions of type 1 membrane proteins such as CD8alpha, CD8beta, CD4, CD28, CD28T, 4-1BB, and CD7, which may be wild-type hinge regions from these molecules or may be altered.

### B. Expression of CAR Constructs

As used herein, an isolated nucleic acid is intended to mean a DNA molecule which can be transformed or introduced into a host cell, preferably a γδ T-cell, and be transcribed and translated to produce a product (e.g., a CAR as herein described). In the isolated nucleic acids of the present invention, a promoter is operably linked to the nucleic acid sequence encoding the CAR of the present invention, i.e., they are positioned so as to promote transcription of the messenger RNA from the DNA encoding the CAR. The term “operatively linked” refers to a juxtaposition wherein the components so described are in a relationship permitting them to function in their intended manner.

The promoter can be of genomic origin or synthetically generated. A variety of promoters for use in host cells relevant to the present disclosure are well-known in the art (e.g., the CD4 promoter disclosed by Marodon, et al. (2003) Blood 101(9):3416-23). The promoter can be constitutive or inducible, where induction is associated with the specific cell type or a specific level of maturation. Alternatively, a number of well-known viral promoters may also be suitable. Promoters of interest include the β-actin promoter, SV40 early and late promoters, immunoglobulin promoter, human cytomegalovirus promoter, retrovirus promoter, and the Friend spleen focus-forming virus promoter. The promoters may or may not be associated with enhancers, wherein the enhancers may be naturally associated with the particular promoter or associated with a different promoter. In embodiments, expression of a CAR polypeptide is under the control of an inducible promoter, for example a promoter that is inducible by a molecule present in a tumor microenvironment (e.g., TGFβ).

The sequence of the open reading frame encoding the various segments of the CARs of the present disclosure can be obtained from a genomic DNA source, a cDNA source, or can be synthesized (e.g., via PCR), or combinations thereof.

In embodiments, for expression of a CAR of the present invention, the naturally occurring or endogenous transcriptional initiation region of the nucleic acid sequence encoding N-terminal component of CD27 can be used to generate the CAR in the host cell. Alternatively, an exogenous transcriptional initiation region can be used which allows for constitutive or inducible expression, wherein expression can optionally be controlled depending upon the host cell, the level of expression desired, the nature of the host cell, and the like.

A termination region encoding a C-terminal component of the CAR can be included. Generally speaking, the source of the termination region is not considered to be critical to the expression of a recombinant protein and a wide variety of termination regions can be employed without adversely affecting expression.

The isolated nucleic acid, which encodes the CAR according to this invention can be prepared in conventional ways. Sequences (natural or synthetic) are isolated and manipulated, as appropriate, so as to allow for the proper joining of the various components. Thus, various nucleic acid sequences encoding for the various segments of the CAR can be isolated, e.g., by employing the polymerase chain reaction (PCR), using appropriate primers. If necessary, specific primers can be designed which result in deletion of undesired portions of a nucleic acid sequence being used as a template. Additionally or alternatively, restriction digests of cloned genes can be used to generate the isolated nucleic acid constructs of the present disclosure. In either case, the sequences can be selected to provide for restriction sites which are blunt-ended, or have complementary overlaps for facilitating incorporation into various vectors. In examples, modification of a nucleic acid sequence (e.g., to introduce one or more point mutations, insertions or deletions), is performed. In one embodiment with regard to CARs of the present disclosure, the modification can, for example, comprise an amino acid change at position R58 of SEQ ID NO: 35. Methods for introducing modifications into nucleic acid sequences are known in the art and can include the use of various kits available for purchase (e.g., QuickChange Site Directed Mutagenesis Kit, Agilent, Santa Clara, CA).

The various manipulations for preparing the isolated nucleic acid encoding a CAR of the present disclosure can be carried out in vitro. In particular embodiments a sequence encoding a CAR is introduced into vectors for cloning and expression in an appropriate host cell using standard transformation or transfection methods. Thus, after each manipulation, the resulting construct from joining of the DNA sequences is cloned, the vector isolated, and the sequence screened to insure that the sequence encodes the desired CAR. The sequence can be screened by restriction analysis, sequencing, or the like.

It is contemplated that the isolated nucleic acid can be introduced into host cells as naked DNA or in a suitable vector. Many suitable vectors are known to those skilled in molecular biology, the choice of which would depend on the function desired and include plasmids, cosmids, viruses, bacteriophages and other vectors used conventionally in genetic engineering. Methods that are well known to those skilled in the art can be used to construct various plasmids and vectors; see, for example, the techniques described in Sambrook et al. (1989) and Ausubel, Current Protocols in Molecular Biology, Green Publishing Associates and Wiley Interscience, N.Y. (1989), (1994). Alternatively, the polynucleotides and vectors of the disclosure can be reconstituted into liposomes for delivery to target cells.

Methods of stably transfecting host cells by electroporation using naked DNA are known in the art (see for example U.S. Pat. No. 6,410,319 disclosing T cell transfection). Naked DNA generally refers to the DNA encoding a CAR of the present invention contained in a plasmid expression vector in proper orientation for expression. Advantageously, the use of naked DNA reduces the time required to produce host cells expressing a CAR of the present invention.

Alternatively, a viral vector (e.g., a retroviral vector, adenoviral vector, adeno-associated viral vector, or lentiviral vector) can be used to introduce the isolated nucleic acid encoding a CAR of the present invention into host cells. Suitable vectors for use in accordance with the method of the present invention are non-replicating in the T-cells. A large number of vectors are known which are based on viruses, where the copy number of the virus maintained in the cell is low enough to maintain the viability of the cell. Illustrative vectors include the pFB-neo vectors (STRATAGENE®) as well as vectors based on HIV, SV40, EBV, HSV or BPV.

Thus, it may be understood that in some embodiments, the isolated nucleic acid is a circular nucleic acid. In some embodiments, the isolated nucleic acid is a vector, such as a plasmid vector, an adenoviral vector, an adeno-associated viral vector, a viral vector, a retroviral vector, (e.g., a gamma retroviral vector), or a lentiviral vector. In some embodiments, the isolated nucleic acid, or an, e.g., contiguous, portion thereof is integrated into the genome of a host cell, such as a host γδ T cell. In an exemplary embodiment, the isolated nucleic acid is retroviral vector.

### C. γδ T Cells

Aspects of the invention include γδ T cells that functionally express an isolated nucleic acid described herein, and thereby expresses a CAR on the surface of the γδ T cell.

Aspects of the invention can additionally or alternatively include γδ T cells having in vitro or in vivo cytotoxic activity against a hematological and/or solid tumor cell that exhibits cell surface expression of CD70. In some cases, the cytotoxic activity is innate activity. In some cases, the cytotoxicity is at least in part, significantly (>about 25%), or entirely, due to the presence of a CAR construct having a binding domain that specifically binds CD70 expressed on the surface of the hematological and/or solid tumor cell. In some cases, the γδ T cells exhibit hematological and/or solid tumor cell killing activity greater than an innate level of in vitro and/or in vivo hematological and/or solid tumor cell killing activity in a control γδ T cell. In some cases, the control γδ T cell does not comprise a CAR construct. In some cases, the control γδ T cell comprises a CAR construct lacking one or more of a binding domain described herein, a hinge region described herein, a transmembrane domain described herein, an intracellular signaling domain described herein, and/or a costimulatory domain described herein.

In some cases, the cytotoxicity is at least in part, significantly (>about 25%), or entirely, due to the presence of a CAR construct having a binding domain that specifically binds CD70 or an epitope within CD70. In some cases, the γδ T cells functionally express a CD70 specific CAR encoded by an isolated nucleic acid described herein.

γδ T cells described herein can exhibit robust and/or persistent hematological and/or solid tumor cell killing activity. In some cases, the hematological and/or solid tumor cell killing activity can persist for at least about 6 days to about 120 days, or for at least about 6 days to about 180 days, from first contact with a hematological and/or solid tumor cell. In some cases, the hematological and/or solid tumor cell killing activity of a γδ T cell described herein, or a progeny thereof, can persist for at least about 6 days to about 120 days, or for at least about 6 days to about 180 days, from first contact with a hematological and/or solid tumor cell, or from administration of the γδ T cell described herein. This persistent hematological and/or solid tumor cell killing activity can be exhibited in vitro, in vivo, or both in vitro and in vivo.

Aspects of the invention can additionally or alternatively include γδ T cells that proliferate in response to contact with cells that exhibit cell surface expression, or overexpression, of CD70. The cells that exhibit cell surface expression of CD70 can be activated immune cells (Bowman et al., J. Immunol. (1994); 152:1756-1761; Nolte et al., Immunol. Rev. (2009); 229:216-231), stromal cells of the thymic medulla, and antigen-presenting cells (Keller et al., Proc Nat Acad Sci USA. (2007); 104:5989-94; Borst et al., Curr Opin Immunol. (2005); 17:275-81; Hendriks et al., J. Immunol. (2005); 175:1665-76). The cells that exhibit cell surface expression, or overexpression, of CD70 can be hematological (Flieswasser et al., Cancers. (2019); 11) and/or solid tumor cells (Pahl et al., Cancer Cell Int. (2015); 15:31; Jilaveanu et al., Hum Pathol. (2012); 43:1394-9; Hishima et al., Int J Cancer. (2002); 98:352-6; Burvenich et al., Theranostics. (2018); 8:4199-209). In some cases, the proliferation is an innate activity. In some cases, the proliferation is at least in part, significantly (>about 20% or >about 25%), or entirely, due to the presence of a CAR construct having a binding domain that specifically binds CD70 expressed on the surface of a cell, for example a hematological and/or solid tumor cell. In some cases, the γδ T cells exhibit a greater level of in vitro and/or in vivo proliferation as compared to a control γδ T cell. In some cases, the control γδ T cell does not comprise a CAR construct. In some cases, the control γδ T cell comprises a CAR construct lacking a binding domain described herein, a hinge region described herein, a transmembrane domain described herein, an intracellular signaling domain described herein, and/or a costimulatory domain described herein.

In some cases, the proliferation is at least in part, significantly (>about 20 or >about 25%), or entirely, due to the presence of a CAR construct having a binding domain that specifically binds CD70 or an epitope within CD70. In some cases, γδ T cells exhibiting proliferation in response to contact with a cell, for example a hematological and/or solid tumor cell that exhibits cell surface expression of CD70 functionally express a CD70 specific CAR encoded by an isolated nucleic acid described herein.

γδ T cells described herein can exhibit robust and/or persistent proliferation in a host organism that comprises a cell, for example a hematological and/or solid tumor cell, that exhibits cell surface expression, or overexpression, of CD70. In some cases, the proliferation can persist for at least about 6 days to about 120 days, or for at least about 6 days to about 180 days, from first contact with a CD70-expressing cell, for example a hematological and/or solid tumor cell, or from a date of administration of the γδ T cell to the host organism. In some cases, the proliferation of a γδ T cell described herein, or a progeny thereof, in the host organism that comprises the hematological and/or solid tumor cell that exhibits cell surface expression, or overexpression, of CD70 can persist for at least about 6 days to about 120 days, or for at least about 6 days to about 180 days, from first contact with a CD70-expressing cell, for example a hematological and/or solid tumor cell, or from the date of first administration of the γδ T cell to the host organism. In some cases, the proliferation in the host organism is at least in part, significantly (>about 20% or >about 25%), or entirely, due to the presence of a CAR construct having a binding domain that specifically binds CD20 or an epitope within CD20. In some cases, γδ T cells exhibiting proliferation in the host organism comprising a cell, for example a hematological and/or solid tumor cell, that exhibits cell surface expression of CD70 functionally express a CD70 specific CAR encoded by an isolated nucleic acid described herein.

In some embodiments, the yd T cells described herein express, or persistently express, pro-inflammatory cytokines such as, but not limited to, tumor necrosis factor alpha or interferon gamma after contact with a CD70-expressing cell, for example a hematological and/or solid tumor cell. In some embodiments, the γδ T cells described herein, or progeny thereof, express, or persistently express, pro-inflammatory cytokines such as tumor necrosis factor alpha or interferon gamma after contact with the CD70-expressing cell, e.g., in a host organism comprising a CD70-expressing hematological and/or solid tumor cell.

In some embodiments, the γδ T cell, or a pharmaceutical composition containing the γδ T cell, exhibits essentially no, or no graft versus host response when introduced into an allogeneic host. In some embodiments, the γδ T cell, or a pharmaceutical composition containing the γδ T cell, exhibits a clinically acceptable level of graft versus host response when introduced into an allogeneic host. In some embodiments, a clinically acceptable level is an amount of graft versus host response that does not require cessation of a γδ T cell treatment to achieve a therapeutically effective treatment. In some embodiments, a clinically acceptable level of graft versus host response (GvHD) is an acute response that is less severe than Grade C according to an applicable International Bone Marrow Transplant Registry (IBMTR) grading scale. The severity of acute graft versus host response is determined by an assessment of the degree of involvement of the skin, liver, and gastrointestinal tract. The stages of individual organ involvement are combined to produce an overall grade, which has prognostic significance. Grade I(A) GvHD is characterized as mild disease, grade II(B) GvHD as moderate, grade III(C) as severe, and grade IV(D) life-threatening. The IBMTR grading system defines the severity of acute GvHD as follows (Rowlings et al., Br J Haematol 1997; 97:855):
Grade A—Stage 1 skin involvement alone (maculopapular rash over <25 percent of the body) with no liver or gastrointestinal involvement
Grade B—Stage 2 skin involvement; Stage 1 to 2 gut or liver involvement
Grade C—Stage 3 involvement of any organ system (generalized erythroderma; bilirubin 6.1 to 15.0 mg/dL; diarrhea 1500 to 2000 mL/day)
Grade D—Stage 4 involvement of any organ system (generalized erythroderma with bullous formation; bilirubin >15 mg/dL; diarrhea >2000 mL/day OR pain OR ileus).
   See also, Schoemans et al., Bone Marrow Transplantation volume 53, pages 1401-1415 (2018), e.g., at Tables 1 and 2, which discloses criteria for assessing and grading acute GvHD.

In some embodiments, the γδ T cell, or a pharmaceutical composition containing the γδ T cell, exhibits reduced or substantially reduced graft versus host response when introduced into an allogeneic host as compared to a graft versus host response exhibited by control αβ T cells, or a control pharmaceutical composition comprising the control αβ T cells, administered to an allogeneic host. In some cases, the control αβ T cell is an allogeneic non-engineered control αβ T cell. In some cases, the control αβ T cell does not comprise a CAR or does not comprise the same CAR as a reference γδ T cell.

The γδ T cells described herein can be δ1, δ2, δ3, or δ4 γδ T cells, or combinations thereof. In some cases, the γδ T cells are mostly (>50%), substantially (>90%), essentially all, or entirely δ2⁻ γδ T cells. In some cases, the γδ T cells are mostly (>50%), substantially (>90%), essentially all, or entirely 81 γδ T cells.

In some embodiments, γδ T cells described herein can be modified to comprise one or more gene edits. Discussed herein, gene editing is a type of genetic engineering in which nucleotide(s)/nucleic acid(s) is/are inserted, deleted, and/or substituted in a DNA sequence, such as the genome of a γδ T cell. Targeted gene editing enables insertion, deletion, and/or substitution at pre-selected sites in the genome of a targeted cell. When a sequence of an endogenous gene is edited, for example by deletion, insertion, or substitution of nucleotide(s)/nucleic acid(s), the endogenous gene comprising the affected sequence may be knocked-out or knocked-down due to the sequence alteration. Therefore, targeted editing may be used to disrupt endogenous gene expression. Discussed herein, a “disrupted gene” refers to a gene comprising an insertion, deletion or substitution relative to an endogenous gene such that expression of a functional protein from the endogenous gene is reduced or inhibited. As used herein, “disrupting a gene” refers to a method of inserting, deleting, or substituting at least one nucleotide/nucleic acid in an endogenous gene such that expression of a functional protein from the endogenous gene is reduced or inhibited. Methods of disrupting a gene are known to those of skill in the art, and described, e.g., in U.S. Pat. No. 11,254,912, incorporated herein by reference in its entirety.

In embodiments, a nuclease-dependent approach can be used to conduct targeted gene editing of a T cell. Such a nuclease-dependent approach can achieve targeted editing through the specific introduction of double strand breaks (DSBs) by specific endonucleases. Such nuclease-dependent targeted editing utilizes DNA repair mechanisms, for example, non-homologous end joining (NHEJ), which occurs in response to DSBs. DNA repair by NHEJ often leads to random insertions or deletions (indels) of a small number of endogenous nucleotides. In contrast to NHEJ mediated repair, repair can also occur by a homology directed repair (HDR). When a donor template containing exogenous genetic material flanked by a pair of homology arms is present, the exogenous genetic material can be introduced into the genome by HDR, which results in targeted integration of the exogenous genetic material. Available endonucleases capable of introducing specific and targeted DSBs include, but are not limited to, zinc-finger nucleases (ZFN), transcription activator-like effector nucleases (TALEN), and RNA-guided CRISPR-Cas9 nuclease (CRISPR/Cas9; Clustered Regular Interspaced Short Palindromic Repeats Associated 9). Discussed herein, a CRISPR system, or CRISPR nuclease system can include a non-coding RNA molecule (e.g., guide RNA) that binds DNA and Cas proteins (e.g., Cas9) with nuclease functionality (Sander et al., Nature Biotechnology (2014); 32:347-355; Hsu et al., Cell (2014); 157(6):1262-1278).

In embodiments, a γδ T cell comprises one or more disrupted genes. For example, one or more genes whose expression is disrupted can comprise adenosine A2a receptor (ADORA), CD276, V-set domain containing T cell activation inhibitor 1 (VTCN1), B and T lymphocyte associated (BTLA), cytotoxic T-lymphocyte-associated protein 4 (CTLA4), indoleamine 2,3-dioxygenase 1 (IDO1), killer cell immunoglobulin-like receptor, three domains, long cytoplasmic tall, 1 (KIR3DL1), lymphocyte-activation gene 3 (LAG3), programmed cell death 1 (PD-1), hepatitis A virus cellular receptor 2 (HAVCR2), V-domain immunoglobulin suppressor of T-cell activation (VISTA), natural killer cell receptor 2B4 (CD244), cytokine inducible SH2-containing protein (CISH), hypoxanthine phosphoribosyltransferase 1 (HPRT), adeno-associated virus integration site (AAVS SITE (E.G. AAVS1, AAVS2, ETC.)), or chemokine (C-C motif) receptor 5 (gene/pseudogene) (CCRS), CD160 molecule (CD160), T-cell immunoreceptor with Ig and ITIM domains (TIGIT), CD96 molecule (CD96), cytotoxic and regulatory T-cell molecule (CRTAM), leukocyte associated immunoglobulin like receptor 1 (LAIR1), sialic acid binding Ig like lectin 7 (SIGLEC7), sialic acid binding Ig like lectin 9 (SIGLEC9), tumor necrosis factor receptor superfamily member 10b (TNERSF10B), tumor necrosis factor receptor superfamily member 10a (TNFRSF10A), caspase 8 (CASP8), caspase 10 (CASP10), caspase 3 (CASP3), caspase 6 (CASP6), caspase 7 (CASP7), Fas associated via death domain (FADD), Fas cell surface death receptor (FAS), transforming growth factor beta receptor II (TGFBRII), transforming growth factor beta receptor I (TGFBR1), SMAD family member 2 (SMAD2), SMAD family member 3 (SMAD3), SMAD family member 4 (SMAD4), SKI proto-oncogene (SKI), SKI-like proto-oncogene (SKIL), TGFB induced factor homeobox 1 (TGIF1), interleukin 10 receptor subunit alpha (ILIORA), interleukin 10 receptor subunit beta (IL10RB), heme oxygenase 2 (HMOX2), interleukin 6 receptor (ILGR), interleukin 6 signal transducer (IL6ST), c-sre tyrosine kinase (CSK), phosphoprotein membrane anchor with glycosphingolipid microdomains 1 (PAGI), signaling threshold regulating transmembrane adaptor 1 (SITI), forkhead box P3 (FOXP3), PR domain 1 (PRDM1), basic leucine zipper transcription factor, ATF-like (BATF), guanylate cyclase 1, soluble, alpha 2 (GUCY1A2), guanylate cyclase 1, soluble, alpha 3 (GUCY1A3), guanylate cyclase 1, soluble, beta 2 (GUCY1B2), guanylate cyclase 1, soluble, beta 3 (GUCY1B3), cytokine inducible SH2-containing protein (CISH), prolyl hydroxylase domain (PHD1, PHD2, PHD3) family of proteins, Cbl proto-oncogene B (CBL-B), Zinc Finger Protein 91 (ZFP91), Roquin, CD58, ICAM-1 or any combination thereof.

In a preferred embodiment, a gene whose expression is disrupted is CISH, a negative regulator of TCR signaling. Disruption of the CISH gene may provide a functional advantage over control cells that have an intact CISH gene in improving the sensitivity to certain cytokines (e.g., IL-2/IL-15), increasing T cell proliferation, and/or limiting T cell exhaustion. In some examples, the CISH gene may be disrupted by methods described in Daher M. et al, Targeting a cytokine checkpoint enhances the fitness of armored cord blood CAR-NK cells, Blood. 2021 Feb. 4; 137 (5): 624-636, which is incorporated by reference herein in its entirety. In one example, the CISH gene is disrupted by gene editing using a CRISPR-Cas system comprising one or more guide RNAs comprising the sequences of any one of SEQ ID NOs: 87-90. In another example, the CISH gene is disrupted by gene editing using a CRISPR-Cas system comprising one or more guide RNAs comprising the sequences of any one of SEQ ID NOs: 96-97.

In embodiments, a gene whose expression is disrupted is CBL-B, a negative regulator of T cell activation. Disruption of the CBL-B gene may provide a functional advantage over control cells that have an intact CBL-B gene in enhancing T cell activation. In some examples, the CBL-B gene may be disrupted by methods described in Augustin R. et al., Targeting Cbl-b in cancer immunotherapy, J Immunother Cancer. 2023 February; 11(2):e006007; Hooper K. et al., Knockout of CBLB Greatly Enhances Anti-Tumor Activity of CAR T Cells, Blood (2018) 132 (Supplement 1): 338; and Guo X. et al., CBLB ablation with CRISPR/Cas9 enhances cytotoxicity of human placental stem cell-derived NK cells for cancer immunotherapy, J Immunother Cancer. 2021 March; 9(3):e001975, each of which is incorporated by reference herein in its entirety. In one example, the CBL-B gene is disrupted by gene editing using a CRISPR-Cas system comprising one or more guide RNAs comprising the sequence of any one of SEQ ID NOs: 93-95.

In embodiments, a gene whose expression is disrupted is Roquin (e.g., Roquin-1). Disruption of the Roquin gene may provide a functional advantage over control cells that have an intact Roquin gene in increasing T cell proliferation and enhancing antitumor activity. In some examples, the Roquin gene may be disrupted by methods described in Mai D et al., Combined disruption of T cell inflammatory regulators Regnase-1 and Roquin-1 enhances antitumor activity of engineered human T cells, Proc Natl Acad Sci USA. 2023 Mar. 21; 120(12):e2218632120, which is incorporated by reference herein in its entirety.

In embodiments, a gene whose expression is disrupted is ZFP91. Disruption of the ZFP91 gene may provide a functional advantage over control cells that have an intact ZFP91 gene in improving T cell glycolytic fitness and effector function. In some examples, the ZFP91 gene may be disrupted by method described in Wang F. et al., J Clin Invest. 2021 Oct. 1; 131(19):e144318, which is incorporated by reference herein in its entirety. In embodiments, a gene whose expression is disrupted is CD58. In embodiments, a gene whose expression is disrupted is ICAM-1. In some embodiments, both CD58 and ICAM-1 are disrupted. Disruption of the CD58 and/or ICAM-1 genes may provide a functional advantage over control cells that have an intact CD58 and/or ICAM-1 gene in disrupting T cell adhesion and co-stimulatory interactions to reduce Host vs Graft allocytotoxicity. In one example, the ICAM-1 gene may be disrupted as described in Teo H Y et al. IL Dec. 18, 2021 Preactivation Enhances the Antitumor Efficacy of Expanded γδT Cells and Overcomes Resistance to Anti-PD-L1 Treatment, Cancer Immunol Res. 2023 Jul. 5; 11(7):978-999, which is incorporated by reference in its entirety. In one example, the ICAM-1 gene is disrupted by gene editing using an RNA-guided nuclease system (e.g., CRISPR-Cas or CRISPR-Mad7 system) comprising one or more guide RNAs comprising the sequences of any one of SEQ ID NOs: 103-104. In one example, the CD58 gene is disrupted by gene editing using an RNA-guided nuclease system (e.g., CRISPR-Cas or CRISPR-Mad7 system) comprising one or more guide RNAs comprising the sequences of any one of SEQ ID NOs: 105-106.

γδ T cells can be obtained from an allogeneic or an autologous donor. The γδ T cells can be, partially or entirely purified, or not purified, and expanded ex vivo. Methods and compositions for ex vivo expansion include, without limitation, those described in WO 2017/197347. The expansion may be performed before or after, or before and after, a CAR construct is introduced into the γδ T cell(s).

In some embodiments, the γδ T cells can be modified to include a nucleic acid construct that encodes a protein which imparts a desired functionality to the γδ T cells of the present disclosure. For example and without limitation, such a nucleic acid may encode for a chimeric DAP10 adaptor polypeptide, described in U.S. Provisional Application No. 63/272,613 and U.S. Provisional Application No. 63/347,194, the contents of each of which is hereby expressly incorporated herein by reference in their entirety. In some embodiments, a chimeric DAP10 adaptor polypeptide is capable of associating with a chimeric antigen receptor of the present disclosure, for example a CAR of the present disclosure may comprise a DAP10-interacting domain. In additional or alternative examples, a chimeric DAP10 adaptor polypeptide may not associate with a CAR of the present disclosure, but instead may interact with an endogenous or exogenous polypeptide that includes a DAP10-interacting domain.

γδ T cells described herein can be stored, e.g., cryopreserved, for use in adoptive cell transfer.

### D. Methods of Inhibiting or Killing CD70-Expressing Cells

Broadly speaking cells targeted by CARs of the present disclosure may be any kind of cell that comprises a CD70 antigen, including cancer cells. In specific embodiments said cancer cells are hematological malignant cells. In specific embodiments, said cancer cells are solid malignant cells. In certain aspects, said cancer cells are lymphoma cells, renal cell carcinoma cells, or gliobastoma cells, for example. In some aspects the cancer cells are HTLV-1-associated malignant cells or EBV-associated malignant cells, for example. In specific embodiments, the cancer cells are CD70-positive. In specific embodiments, the cancer being treated is renal cell cancer, thymic carcinoma, nasopharyngeal carcinoma, brain tumor, Hodgkin and non-Hodgkin lymphomas, Waldenstrom's macroglobulinemia, chronic lymphocytic leukemia, T-cell leukemia, multiple myeloma, EBV- and HTLV-I associated malignancies, kidney, pancreatic, larynx, pharynx, melanoma, ovarian, lung (including lung adenocarcinoma), colon, breast, or brain.

In specific embodiments of the invention, the T cell comprising the chimeric receptor targets any cell that comprises a CD70 antigen, whether or not that targeted cell is cancerous. For example, in some embodiments CD70 is expressed on cells that are related to autoimmune disorders, as in certain aspects associated with the invention there is dysregulation of CD70-CD27 co-stimulation that contributes to autoimmunity. In specific embodiments, the CD70 cells are present in an individual with an autoimmune disorder such as rheumatoid arthritis (RA), arthritis (including psoriatic arthritis), inflammation, autoimmune encephalitis, inflammatory bowel disease, colitis, and lupus.

One or multiple non-engineered γδ T-cell populations, engineered γδ T-cell populations, and/or admixtures thereof, having cytotoxic activity against a CD70-expressing cell, for example a CD70-expressing hematological and/or solid tumor cells, can be administered to a subject in any order or simultaneously. If simultaneously, the multiple non-engineered γδ T-cell population, engineered γδ T-cell population, and/or admixtures thereof, of the invention can be provided in a single, unified form, such as an intravenous injection, or in multiple forms, for example, as multiple intravenous infusions, s.c, injections or pills. The non-engineered γδ T-cell population, engineered γδ T-cell population, and/or admixtures thereof, of the invention can be packed together or separately, in a single package or in a plurality of packages. One or all of the non-engineered γδ T-cell population, engineered γδ T-cell population, and/or admixtures thereof, of the invention can be given in multiple doses. If not simultaneous, the timing between the multiple doses may vary to as much as about a week, a month, two months, three months, four months, five months, six months, or about a year. In some cases, a non-engineered, enriched γδ T-cell population, an engineered, enriched γδ T-cell population, and/or admixtures thereof, of the invention can proliferate within a subject's body, in vivo, after administration to a subject. One or more non-engineered γδ T-cell populations, one or more engineered γδ T-cell populations, and/or admixtures thereof, can be frozen to provide cells for multiple treatments with the same cell preparation. One or more non-engineered γδ T-cell populations, one or more engineered γδ T-cell populations, and/or admixtures thereof, of the disclosure, and pharmaceutical compositions comprising the same, can be packaged as a kit. A kit may include instructions (e.g., written instructions) on the use of the non-engineered γδ T-cell population, the engineered γδ T-cell population, and/or admixtures thereof, and compositions comprising the same.

In some cases, a method of treating a CD70-expressing disorder comprises administering to a subject a therapeutically effective amount of a non-engineered γδ T-cell population, an engineered γδ T-cell population, and/or admixtures thereof, wherein the administration treats the CD70-expressing disease or condition (e.g., cancer). In some embodiments the therapeutically effective amount of the non-engineered γδ T-cell population, the engineered γδ T-cell population, and/or admixtures thereof, is administered for at least about 10 seconds, 30 seconds, 1 minute, 10 minutes, 30 minutes, 1 hour, 2 hours, 3 hours, 4 hours, 5 hours, δ hours, 12 hours, 24 hours, 2 days, 3 days, 4 days, 5 days, 8 days, 1 week, 2 weeks, 3 weeks, 1 month, 2 months, 3 months, 4 months, 5 months, 6 months, or 1 year. In some embodiments the therapeutically-effective amount of the non-engineered γδ T-cell population, the engineered γδ T-cell population, and/or admixtures thereof, is administered for at least one week. In some embodiments the therapeutically-effective amount of the non-engineered γδ T-cell population, the engineered γδ T-cell population, and/or admixtures thereof, is administered for at least two weeks.

A non-engineered γδ T-cell population, an engineered γδ T-cell population, and/or admixtures thereof, described herein can be administered before, during, or after the occurrence of a disease or condition, and the timing of administering a pharmaceutical composition containing the γδ T-cell population can vary. For example, the γδ T-cell population can be used as a prophylactic and can be administered continuously to subjects with a propensity to conditions or diseases in order to lessen a likelihood of the occurrence of the disease or condition. The initial administration can be via any route practical, such as by any route described herein using any formulation described herein. In some examples, the administration of a γδ T-cell population of the disclosure is an intravenous administration. For example, one or multiple dosages of the γδ T-cell population can be administered as soon as is practicable after the onset of a CD70-expressing cancer and for a length of time necessary for the treatment of the disease, such as, for example, from about 24 hours to about 48 hours, from about 48 hours to about 1 week, from about 1 week to about 2 weeks, from about 2 weeks to about 1 month, from about 1 month to about 3 months. In some embodiments, one or multiple dosages of the γδ T-cell population can be administered years after onset of the cancer and before or after other treatments.

In some embodiments, the γδ T-cell population is administered simultaneously or sequentially with one or more methods to elevate common gamma chain cytokine(s). As used herein, “one or more methods to elevate common gamma chain cytokine(s): refers to a method, or combination of methods, that alters the physiological state of a subject, such that at least one common gamma chain cytokine level is elevated in the subject. In some embodiments, the method elevates the level of one or more common gamma chain cytokine(s) selected from the group consisting of IL-2, IL-7, and IL-15, preferably wherein the method elevates the level of IL-15 in the subject. In some embodiments, the method comprises lymphodepletion. In some embodiments, the method comprises administering one or more common gamma chain cytokine(s) to the subject. In some cases, IL-2, IL-7, and/or IL-15, preferably IL-15, are administered. In some embodiments, the method comprises secreting common gamma chain cytokine(s) from an administered, e.g., γδ T cell. In some cases, IL-2, IL-7, and/or IL-15, preferably IL-15, are secreted.

In some embodiments, the administering one or more methods to elevate common gamma chain cytokine(s) comprises lymphodepletion before introducing the γδ T cell(s). In some embodiments, the administering one or more methods to elevate common gamma chain cytokine(s) comprises administering simultaneously with introducing the γδ T cell(s) or sequentially an amount of common gamma chain cytokine(s) effective to increase proliferation, cytotoxic activity, persistence, or the combination thereof of the introduced γδ T cell(s), preferably wherein the method comprises administering IL-2 or one or more mimetics thereof, more preferably wherein the method comprises administering IL-15 or one or more mimetics thereof. The amount of administered common gamma chain cytokine(s) can be an amount effective to increase proliferation, cytotoxic activity, persistence, or the combination thereof of the introduced γδ T cell(s) before and/or after introducing the γδ T cell(s). Exemplary amounts of IL-15 include, without limitation between 0.01-10 μg/kg/dose every 24 hours for IL-15. Exemplary amounts of IL-2 include, without limitation, between about 3×10⁶ and about 22×10⁶ units every 8-48 hours. For example, the dosing regimen for IL2 in RCC is 600,000 International Units/kg (0.037 mg/kg) IV q8 hr infused over 15 minutes for a maximum 14 doses.

In some embodiments, the administering one or more methods to elevate common gamma chain cytokine(s) comprises lymphodepletion before administering the γδ T cell(s) and administering simultaneously with introducing the γδ T cell(s) or sequentially an amount of common gamma chain cytokine(s) effective to increase proliferation, cytotoxic activity, persistence, or the combination thereof of the introduced γδ T cell(s).

The γδ T cells of the subject invention can also be advantageously administered to a patient in conjunction with (e.g., before, simultaneously or following) any number of relevant treatment modalities (e.g., for treating cancer) including, e.g., chemotherapy, radiation therapy, or immunotherapy. A patient can also be preconditioned with a therapeutically effective amount of γδ T cells prior to receiving the chemotherapy, radiation therapy, or immunotherapy (e.g., cell therapy). Suitable immunotherapies for use in combination with γδ T cells include autologous and allogeneic cell therapies, engineered T and NK cells, immune engagers, fusion proteins, or other immune-oncology agents.

In some embodiments, the subject γδ T cells can be administered in conjunction with an appropriate cellular immunotherapy for treating a disease such as cancer (e.g. CAR T or CAR NK cells, or Treg therapy). For example, a method of treatment (e.g., for treating cancer) according to the present invention can include a conditioning step, e.g. a preconditioning step, of administering a therapeutically effective amount of γδ T cells to a subject simultaneously or sequentially with administration of a cellular immunotherapy directed against the disease (e.g., cancer). In embodiments, the cellular immunotherapy can comprise further administering an engineered T cell or NK cell therapy comprising a CAR binding to any tumor associated antigen of interest. In embodiments, the subject γδ T cells may comprise a dual CAR binding to CD70 and another tumor-associated antigen.

In embodiments, the γδ T cells of the subject invention may be advantageously administered in conjunction with adoptive cell therapies (ACT) (for reviews of HSCT and adoptive cell therapy approaches, see, Rager & Porter, Ther Adv Hematol (2011) 2(6) 409-428; Roddie & Peggs, Expert Opin. Biol. Ther (2011) 11(4):473-487; Wang et al. Int. J. Cancer: (2015) 136, 1751-1768; and Chang, Y. J. and X. J. Huang, Blood Rev, 2013. 27(1): 55-62), each of which is incorporated by reference herein in its entirety. Such adoptive cell therapies include, but are not limited to, allogeneic and autologous hematopoietic stem cell transplantation, donor leukocyte (or lymphocyte) infusion (DLI), adoptive transfer of tumor infiltrating lymphocytes, or adoptive transfer of T cells or NK cells (including recombinant cells, e.g., CAR T, CAR NK). Beyond the necessity for donor-derived cells to reconstitute hematopoiesis after radiation and chemotherapy, immunology reconstitution from transferred cells is important for the elimination of residual tumor cells. The efficacy of ACT as a curative option for malignancies is influenced by a number of factors including the origin, composition and phenotype (lymphocyte subset, activation status) of the donor cells, the underlying disease, the pre-transplant conditioning regimen and post-transplant immune support (e.g., IL-2 therapy) and the graft-versus-tumor (GVT) effect mediated by donor cells within the graft. Additionally, these factors may be balanced against transplant-related mortality, typically arising from the conditioning regimen and/or excessive immune activity of donor cells within the host (i.e., graft-versus-host disease, cytokine release syndrome, etc.).

### E. Methods of CAR Detection

Quantification of CAR transgene level in host cells of the present disclosure can be accomplished in a variety of ways. Examples include but are not limited to genomic detection (e.g., quantitative PCR, digital PCR), transcriptomic detection (e.g., RNA sequencing, RNA in situ hybridization), proteomic detection (e.g., fluorescence microscopy), and organismal detection (e.g., positron emission tomography (PET) scanning, two-photon microscopy), and various combinations thereof.

As discussed above, in embodiments, an isolated nucleic acid encoding a CAR polypeptide may include one or more labels or markers to facilitate an ability to monitor CAR expression level. However, it is herein recognized that inclusion of a label or marker, for example LNGFR, can disadvantageously impact viral titer levels given the increased molecular weight of the resultant CAR, particularly in a case where other polypeptides are also associated with the CAR (e.g., dnTGFβR2). Other additional or alternative methodology, for example reliance on vector copy number (VCN) is also associated with disadvantages, including but not limited to the fact that the ratio of VCN to CAR may require substantial process development time and may change throughout scale up/process refinement.

In one embodiment, quantification of a CAR may be determined via immunological detection of a linker region (e.g., T2A, P2A, E2A, F2A), for example a cleavage linker region that couples a CD27 domain followed by one or more costimulatory and/or intracellular domains to an additional polypeptide (e.g., dnTGFβR2). An advantage of such an approach is that the linker region is directly coupled to the CAR, and can enable intracellular C-terminal detection of said CAR. For example and without limitation, an antibody that can be used to detect 2A peptides includes anti-2A (3H4) (Novus Biologicals, Littleton, CO).

In the case of 2A peptides, the self-cleaving mechanism is the result of ribosome skipping the formation of a glycyl-prolyl peptide bond at the C-terminus of the 2A peptide. Because of efficient cleavage, in preferred embodiments a P2A peptide is used. The efficient cleavage of P2A peptides may reduce or eliminate exogenous epitopes associated with a gene of interest, which in turn may reduce immunogenicity and hence impart a lesser likelihood that a patient treated with a CAR-engineered host cell will exhibit an immune reaction against the engineered host cell (see WO 2020/223478).

### F. Dosages

One or multiple engineered host cell populations, non-engineered cells, and/or admixtures thereof, of the present disclosure, may be formulated in unit dosage forms suitable for single administration of precise dosages. In some cases, the unit dosage forms comprise additional lymphocytes. In unit dosage form, the formulation is divided into unit doses containing appropriate quantities of one or more compounds. The unit dosage can be in the form of a package containing discrete quantities of the formulation. Non-limiting examples are packaged tablets or capsules, and powders in vials or ampoules. Aqueous suspension compositions can be packaged in single-dose non-reclosable containers. Multiple-dose re-closable containers can be used, for example, in combination with a preservative or without a preservative. In some examples, the pharmaceutical composition does not comprise a preservative. Formulations for parenteral injection can be presented in unit dosage form, for example, in ampoules, or in multi-dose containers with a preservative.

One or multiple engineered host cell populations, non-engineered cells, and/or admixtures thereof, of the present disclosure, may be present in a composition in an amount of at least 5 cells, at least 10 cells, at least 20 cells, at least 30 cells, at least 40 cells, at least 50 cells, at least 60 cells, at least 70 cells, at least 80 cells, at least 90 cells, at least 100 cells, at least 200 cells, at least 300 cells, at least 400 cells, at least 500 cells, at least 600 cells, at least 700 cells, at least 800 cells, at least 900 cells, at least 1×10³ cells, at least 2×10³ cells, at least 3×10³ cells, at least 4×10³ cells, at least 5×10³ cells, at least 6×10³ cells, at least 7×10³ cells, at least 8×10³ cells, at least 9×10³ cells, at least 1×10⁴ cells, at least 2×10⁴ cells, at least 3×10⁴ cells, at least 4×10⁴ cells, at least 5×10⁴ cells, at least 6×10⁴ cells, at least 7×10⁴ cells, at least 8×10⁴ cells, at least 9×10⁴ cells, at least 1×10⁵ cells, at least 2×10⁵ cells, at least 3×10⁵ cells, at least 4×10⁵ cells, at least 5×10⁵ cells, at least 6×10⁵ cells, at least 7×10⁵ cells, at least 8×10⁵ cells, at least 9×10⁵ cells, at least 1×10⁶ cells, at least 2×10⁶ cells, at least 3×10⁶ cells, at least 4×10⁶ cells, at least 5×10⁶ cells, at least 6×10⁶ cells, at least 7×10⁶ cells, at least 8×10⁶ cells, at least 9×10⁶ cells, at least 1×10⁷ cells, at least 2×10⁷ cells, at least 3×10⁷ cells, at least 4×10⁷ cells, at least 5×10⁷ cells, at least 6×10⁷ cells, at least 7×10⁷ cells, at least 8×10⁷ cells, at least 9×10⁷ cells, at least 1×10⁸ cells, at least 2×10⁸ cells, at least 3×10⁸ cells, at least 4×10⁸ cells, at least 5×10⁸ cells, at least 6×10⁸ cells, at least 7×10⁸ cells, at least 8×10⁸ cells, at least 9×10⁸ cells, at least 1×10⁹ cells, or more.

The therapeutically effective dose of one or multiple engineered host cell populations, non-engineered cells, and/or admixtures thereof, of the invention can be from about 1 cell to about 10 cells, from about 1 cell to about 100 cells, from about 1 cell to about 10 cells, from about 1 cell to about 20 cells, from about 1 cell to about 30 cells, from about 1 cell to about 40 cells, from about 1 cell to about 50 cells, from about 1 cell to about 60 cells, from about 1 cell about 70 cells, from about 1 cell to about 80 cells, from about 1 cell to about 90 cells, from about 1 cell to about 100 cells, from about 1 cell to about 1×10³ cells, from about 1 cell to about 2×10³ cells, from about 1 cell to about 3×10³ cells, from about 1 cell to about 4×10³ cells, from about 1 cell to about 5×10³ cells, from about 1 cell to about 6×10³ cells, from about 1 cell to about 7×10³ cells, from about 1 cell to about 8×10³ cells, from about 1 cell to about 9×10³ cells, from about 1 cell to about 1×10⁴ cells, from about 1 cell to about 2×10⁴ cells, from about 1 cell to about 3×10⁴ cells, from about 1 cell to about 4×10⁴ cells, from about 1 cell to about 5×10⁴ cells, from about 1 cell to about 6×10⁴ cells, from about 1 cell to about 7×10⁴ cells, from about 1 cell to about 8×10⁴ cells, from about 1 cell to about 9×10⁴ cells, from about 1 cell to about 1×10⁵ cells, from about 1 cell to about 2×10⁵ cells, from about 1 cell to about 3×10⁵ cells, from about 1 cell to about 4×10⁵ cells, from about 1 cell to about 5×10⁵ cells, from about 1 cell to about 6×10⁵ cells, from about 1 cell to about 7×10⁵ cells, from about 1 cell to about 8×10⁵ cells, from about 1 cell to about 9×10⁵ cells, from about 1 cell to about 1×10⁶ cells, from about 1 cell to about 2×10⁶ cells, from about 1 cell to about 3×10⁶ cells, from about 1 cell to about 4×10⁶ cells, from about 1 cell to about 5×10⁶ cells, from about 1 cell to about 6×10⁶ cells, from about 1 cell to about 7×10⁶ cells, from about 1 cell to about 8×10⁶ cells, from about 1 cell to about 9×10⁶ cells, from about 1 cell to about 1×10⁷ cells, from about 1 cell to about 2×10⁷ cells, from about 1 cell to about 3×10⁷ cells, from about 1 cell to about 4×10⁷ cells, from about 1 cell to about 5×10⁷ cells, from about 1 cell to about 6×10⁷ cells, from about 1 cell to about 7×10⁷ cells, from about 1 cell to about 8×10⁷ cells, from about 1 cell to about 9×10⁷ cells, from about 1 cell to about 1×10⁸ cells, from about 1 cell to about 2×10⁸ cells, from about 1 cell to about 3×10⁸ cells, from about 1 cell to about 4×10⁸ cells, from about 1 cell to about 5×10⁸ cells, from about 1 cell to about 6×10⁸ cells, from about 1 cell to about 7×10⁸ cells, from about 1 cell to about 8×10⁸ cells, from about 1 cell to about 9×10⁸ cells, or from about 1 cell to about 1×10⁹ cells.

In some cases, the therapeutically effective dose of therapeutically effective dose of one or multiple engineered host cell populations, non-engineered cells, and/or admixtures thereof, of the invention can be from about 1×10³ cells to about 2×10³ cells, from about 1×10³ cells to about 3×10³ cells, from about 1×10³ cells to about 4×10³ cells, from about 1×10³ cells to about 5×10³ cells, from about 1×10³ cells to about 6×10³ cells, from about 1×10³ cells to about 7×10³ cells, from about 1×10³ cells to about 8×10³ cells, from about 1×10 cells to about 9×10³ cells, from about 1×10 cells to about 1×10⁴ cells, from about 1×10³ cells to about 2×10⁴ cells, from about 1×10³ cells to about 3×10⁴ cells, from about 1×10³ cells to about 4×10⁴ cells, from about 1×10³ cells to about 5×10⁴ cells, from about 1×10³ cells to about 6×10⁴ cells, from about 1×10³ cells to about 7×10⁴ cells, from about 1×10³ cells to about 8×10⁴ cells, from about 1×10³ cells to about 9×10⁴ cells, from about 1×10³ cells to about 1×10⁵ cells, from about 1×10³ cells to about 2×10⁵ cells, from about 1×10³ cells to about 3×10⁵ cells, from about 1×10³ cells to about 4×10⁵ cells, from about 1×10³ cells to about 5×10⁵ cells, from about 1×10³ cells to about 6×10⁵ cells, from about 1×10³ cells to about 7×10⁵ cells, from about 1×10³ cells to about 8×10⁵ cells, from about 1×10³ cells to about 9×10⁵ cells, from about 1×10³ cells to about 1×10⁶ cells, from about 1×10³ cells to about 2×10⁶ cells, from about 1×10³ cells to about 3×10⁶ cells, from about 1×10³ cells to about 4×10⁶ cells, from about 1×10³ cells to about 5×10⁶ cells, from about 1×10³ cells to about 6×10⁶ cells, from about 1×10³ cells to about 7×10⁶ cells, from about 1×10³ cells to about 8×10⁶ cells, from about 1×10³ cells to about 9×10⁶ cells, from about 1×10³ cells to about 1×10⁷ cells, from about 1×10³ cells to about 2×10⁷ cells, from about 1×10³ cells to about 3×10⁷ cells, from about 1×10³ cells to about 4×10⁷ cells, from about 1×10³ cells to about 5×10⁷ cells, from about 1×10³ cells to about 6×10⁷ cells, from about 1×10³ cells to about 7×10⁷ cells, from about 1×10³ cells to about 8×10⁷ cells, from about 1×10³ cells to about 9×10⁷ cells, from about 1×10³ cells to about 1×10⁸ cells, from about 1×10³ cells to about 2×10⁸ cells, from about 1×10³ cells to about 3×10⁸ cells, from about 1×10³ cells to about 4×10⁸ cells, from about 1×10³ cells to about 5×10⁸ cells, from about 1×10 cells to about 6×10⁸ cells, from about 1×10 cells to about 7×10⁸ cells, from about 1×10³ cells to about 8×10⁸ cells, from about 1×10³ cells to about 9×10⁸ cells, or from about 1×10³ cells to about 1×10⁹ cells.

In some cases, the therapeutically effective dose of one or multiple engineered host cell populations, non-engineered cells, and/or admixtures thereof, of the invention can be from about 1×10⁶ cells to about 2×10⁶ cells, from about 1×10⁶ cells to about 3×10⁶ cells, from about 1×10⁶ cells to about 4×10⁶ cells, from about 1×10⁶ cells to about 5×10⁶ cells, from about 1×10⁶ cells to about 6×10⁶ cells, from about 1×10⁶ cells to about 7×10⁶ cells, from about 1×10⁶ cells to about 8×10⁶ cells, from about 1×10⁶ cells to about 9×10⁶ cells, from about 1×10⁶ cells to about 1×10⁷ cells, from about 1×10⁶ cells to about 2×10⁷ cells, from about 1×10⁶ cells to about 3×10⁷ cells, from about 1×10⁶ cells to about 4×10⁷ cells, from about 1×10⁶ cells to about 5×10⁷ cells, from about 1×10⁶ cells to about 6×10⁷ cells, from about 1×10⁶ cells to about 7×10⁷ cells, from about 1×10⁶ cells to about 8×10⁷ cells, from about 1×10⁶ cells to about 9×10⁷ cells, from about 1×10⁶ cells to about 1×10⁸ cells, from about 1×10⁶ cells to about 2×10⁸ cells, from about 1×10⁶ cells to about 3×10⁸ cells, from about 1×10⁶ cells to about 4×10⁸ cells, from about 1×10⁶ cells to about 5×10⁸ cells, from about 1×10⁶ cells to about 6×10⁸ cells, from about 1×10⁶ cells to about 7×10⁸ cells, from about 1×10⁶ cells to about 8×10⁸ cells, from about 1×10⁶ cells to about 9×10⁸ cells, from about 1×10⁶ cells to about 1×10⁹ cells, from about 1×10⁶ cells to about 2×10⁹ cells, from about 1×10⁶ cells to about 3×10⁹ cells, from about 1×10⁶ cells to about 4×10⁹ cells, from about 1×10⁶ cells to about 5×10⁹ cells, from about 1×10⁶ cells to about 6×10⁹ cells, from about 1×10⁶ cells to about 7×10⁹ cells, from about 1×10⁶ cells to about 8×10⁹ cells, from about 1×10⁶ cells to about 9×10⁹ cells, from about 1×10⁷ cells to about 1×10⁹ cells, from about 1×10⁷ cells to about 2×10⁹ cells, from about 1×10⁷ cells to about 3×10⁹ cells, from about 1×10⁷ cells to about 4×10⁹ cells, from about 1×10⁷ cells to about 5×10⁹ cells, from about 1×10⁷ cells to about 6×10⁹ cells, from about 1×10⁷ cells to about 7×10⁹ cells, from about 1×10⁷ cells to about 8×10⁹ cells, from about 1×10⁷ cells to about 9×10⁹ cells, from about 1×10⁸ cells to about 1×10⁹ cells, from about 1×10⁸ cells to about 2×10⁹ cells, from about 1×10⁸ cells to about 3×10⁹ cells, from about 1×10⁸ cells to about 4×10⁹ cells, from about 1×10⁸ cells to about 5×10⁹ cells, from about 1×10⁸ cells to about 6×10⁹ cells, from about 1×10⁸ cells to about 7×10⁹ cells, from about 1×10⁸ cells to about 8×10⁹ cells, from about 1×10⁸ cells to about 9×10⁹ cells, or from about 1×10⁸ cells to about 1×10¹⁰ cells.

### G. Preservation

In some embodiments, the one or multiple engineered host cell populations, non-engineered cells, and/or admixtures thereof, of the invention may be formulated in freezing media and placed in cryogenic storage units such as liquid nitrogen freezers (−195 C) or ultra-low temperature freezers (−65 C, −80 C or −120 C) for long-term storage of at least about 1 month, 2 months, 3 months, 4 months, 5 months, 6 months, 1 year, 2 years, 3 years, or at least 5 years. The freeze media can contain dimethyl sulfoxide (DMSO), and/or sodium chloride (NaCl), and/or dextrose, and/or dextran sulfate and/or hydrohyethyl starch (HES) with physiological pH buffering agents to maintain pH between about 6.0 to about 6.5, about 6.5 to about 7.0, about 7.0 to about 7.5, about 7.5 to about 8.0 or about 6.5 to about 7.5. In embodiments, the cryopreserved cells can be thawed and further processed, for example by stimulation with antibodies, proteins, peptides, and/or cytokines as mentioned herein. The cryopreserved cells can be thawed and genetically modified with viral vectors (including retroviral and lentiviral vectors) or non-viral means (including RNA, DNA, and proteins) as described herein. Alternatively, host cells as described herein can be, e.g., optionally expanded by the methods described herein, genetically modified, and then cryopreserved.

Thus, genetically engineered and/or non-engineered cells as disclosed herein can be cryopreserved to generate cell banks in quantities of at least about 1, 5, 10, 100, 150, 200, 500 vials at about at least 10¹, 10², 10³, 10⁴, 10⁵, 10⁶, 10⁷, 10⁸, 10⁹, or at least about 10¹⁰ cells per mL in freeze media. The cryopreserved cell banks may retain their functionality and can be thawed and, optionally, be activated/stimulated and/or expanded. In some aspects, thawed cells can be stimulated and expanded in suitable closed vessels such as cell culture bags and/or bioreactors to generate quantities of cells as allogeneic cell product. In other examples, the croyperserved cells comprise an autologous cell product. Cryopreserved cells can maintain their biological functions for at least about 6 months, 7 months, 8 months, 9 months, 10 months, 11 months, 12 months, 13 months, 15 months, 18 months, 20 months, 24 months, 30 months, 36 months, 40 months, 50 months, or at least about 60 months under cryogenic storage condition. In some aspects, no preservatives are used in the formulation. In some embodiments, the cryopreserved cells can be thawed and infused into multiple patients as allogeneic off-the-shelf cell product.

### EXAMPLES

The following examples are put forth so as to provide those of ordinary skill in the art with a complete disclosure and description of how to make and use the methods and compositions of the invention, and are not intended to limit the scope of what the inventors regard as their invention. Efforts have been made to ensure accuracy with respect to numbers used (e.g., amounts, temperature, etc.) but some experimental errors and deviations should be accounted for. Unless indicated otherwise, parts are parts by weight, molecular weight is average molecular weight, temperature is in degrees Centigrade, and pressure is at or near atmospheric.

### Example 1. In Vitro Activity of Vδ1 CD27 Receptor CAR T Cells

This Example demonstrates that Vδ1 CD27 CAR T cells exhibit robust activity against cells expressing CD70 in vitro.

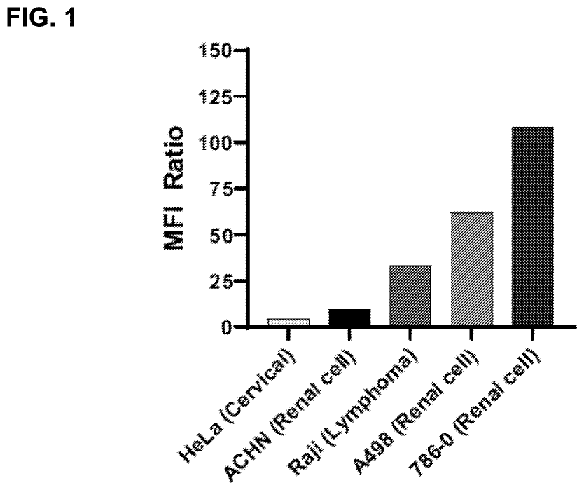

As shown at FIG. 1, target cell lines Raji, HeLa, ACHN, A498, and 786-O (ATCC) were stained with anti-CD70 antibody (Biolegend) or isotype (R&D Systems). Cells were analyzed on the Novocyte (Agilent). CD70 expression in different cell lines was graphed as MFI (mean fluorescence intensity) ratio. The resulting ratio of antibody MFI to isotype MFI was graphed. CD70 low-expressing: HeLa and ACHN, CD70 medium-expressing: Raji, and CD70 high-expressing: A498 and 786-O.

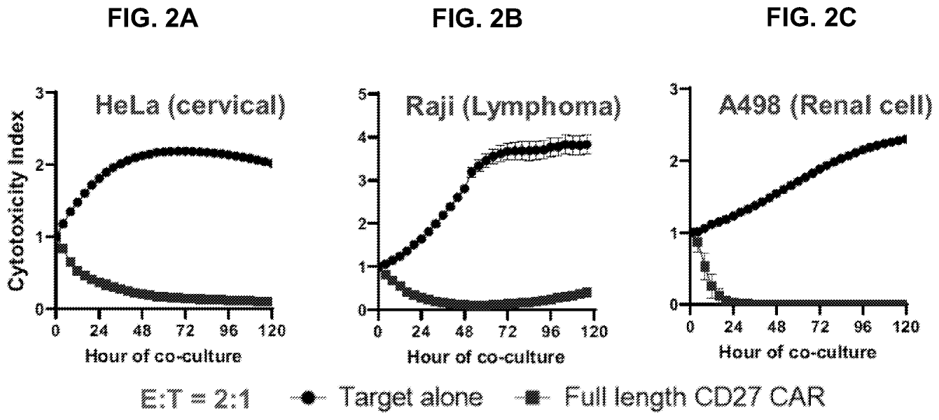

For the long-term cytotoxicity assay, Vδ1 CD27 receptor CAR T cells were cocultured with target cell lines (HeLa, FIG. 2A; Raji, FIG. 2B; A498, FIG. 2C) at an effector to target ratio (E:T) of 2:1 for 120 hours. Target cells were modified to express Nuc-NIR by transducing them with Incucyte® Nuclight NIR lentivirus (Sartorius). Viable Nuc-NIR expressing target cells were quantified every 4 hours by the Incucyte® SX5 (Sartorius). Cytotoxicity index was calculated by dividing the total NIR object area of each time point by the value of time 0 hours. Vδ1 CD27 receptor CAR T cells showed strong cytotoxic activity against different cancer cell lines such as HeLa (cervical), Raji (Lymphoma), and A498 (Renal cell). The lower cytotoxicity index indicates better cytotoxic potential of Vδ1 CD27 receptor CAR T cells (boxes) versus target cells alone (circles).

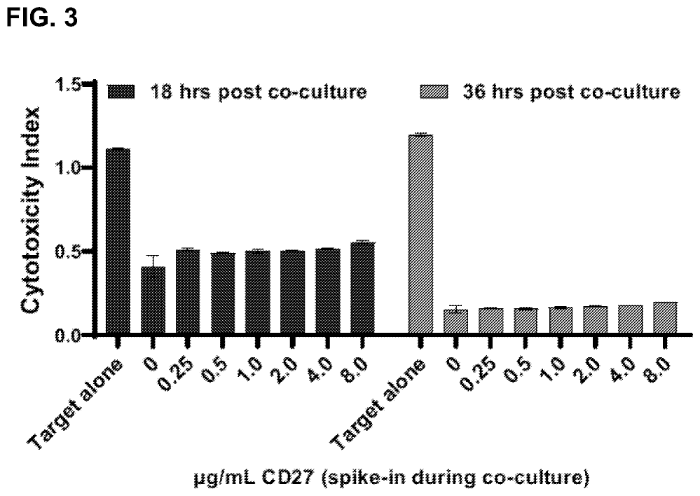

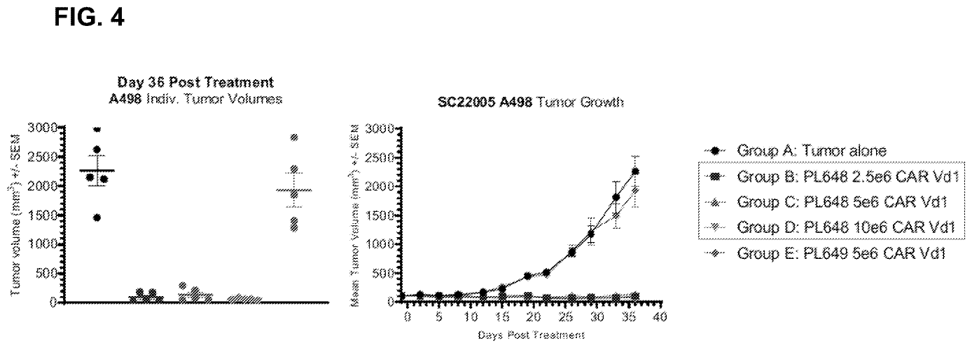

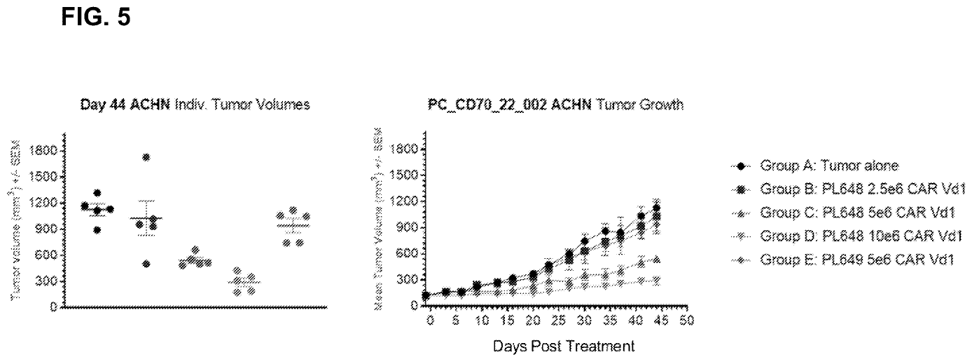

It was assessed as to whether soluble CD27 could impact cytotoxic potential of Vδ1 CD27 receptor CAR T cells. Briefly, Vδ1 CD27 receptor CAR T cells were cocultured with Nuc-NIR expressing A498 target cell line at an E:T ratio of 1:2. At time 0 hours, soluble CD27 (Acro Biosystems) was added to the coculture at different concentrations ranging from 0-8 μg/mL. Viable Nuc-NIR expressing target cells were quantified every 2 hours by the Incucyte® SX5 (Sartorius). Cytotoxicity index at time points 18-hour and 36-hour was calculated by dividing the total NIR object area of each time point by the value at time 0 hours. The lack of difference in the cytotoxic index between co-cultures at 0 μg and 0.25-8 μg/mL of soluble CD27 indicates that the presence of soluble CD27 does not interfere with the cytotoxic potential of Vδ1 CD27 receptor CAR T cells (FIG. 3).

### Example 2. In Vivo Control of Renal Cell Carcinoma by Vδ1 CD27 Receptor CAR T Cells

This Example demonstrates that full-length CD27 CAR with 4-1BB costimulatory domain and CD3ζ signaling domain exhibits improved in vivo tumor control as compared to truncated CD27 CAR with CD8 hinge/TM domain, 4-1BB costimulatory domain, and CD3ζ signaling domain.

The in vivo efficacy of Vδ1 CD27 receptor CAR T cells was assessed in a subcutaneous renal cancer model using A498 cells. Female NSG mice (The Jackson Laboratory) were implanted subcutaneously with 4×10⁶ A498 cells (ATCC), mixed with Matrigel (1:1 ratio) (Corning). Animals were randomized into groups of 5 when tumor volume reached an average of 100 mm³ and were injected IV (tail vein) with 2.5×10⁶, 5×10⁶, or 10×10⁶ Vδ1 CD27 receptor CAR T cells. Negative control groups were treated with non-functioning Vδ1 CD27 truncated receptor CAR T cells at a dose of 5×10⁶ CAR+ cells (Group E) and a tumor alone group (Group A, no treatment). Human IL-2 (Proleukin) was administered intraperitoneally prior to treatment and then thrice a week. Tumor volume was measured twice a week with calipers. Tumor response was durable at all three dose levels of Vδ1 CD27 receptor CAR T cells (Groups B-D) as compared to control groups (tumor alone and the group treated with non-functioning Vδ1 CD27 truncated receptor CAR T cells). These data support the cytotoxic potential of Vδ1 CD27 receptor CAR T cells in a human renal cancer cell xenograft model.

The in vivo efficacy of Vδ1 CD27 receptor CAR T cells was also assessed in a subcutaneous renal cancer model using ACHN cells. Female NSG mice (The Jackson Laboratory) were implanted subcutaneously with 5×10⁶ ACHN cells (ATCC), mixed with Matrigel (1:1 ratio) (Corning). Animals were randomized into groups of 5 when tumor volume reached an average of 120 mm³ and were injected IV (tail vein) with 2.5×10⁶, 5×10⁶, or 10×10⁶ Vδ1 CD27 receptor CAR-T cells. Negative control groups were treated with Vδ1 CD27 truncated receptor CAR-T cells at a dose of 5×10⁶ CAR+ cells (Group E) or left untreated (Group A). Human IL-2 was administered intraperitoneally prior to treatment and then thrice a week. Tumor volume was measured twice a week with calipers. Tumor response was durable at the higher dose levels of Vδ1 CD27 receptor CAR T cells (Group C, Group D) as compared to control groups (tumor alone and the group treated with non-functioning Vδ1 CD27 truncated receptor CAR-T cells). These data support the cytotoxic potential of Vδ1 CD27 receptor CAR-T cells in a human renal cancer cell xenograft model that has low CD70 expression.

### Example 3. Identification of CD27 CAR Expression

This Example demonstrates that antibodies to a 2A peptide included as part of a CD27 CAR can be used to determine CD27 CAR surface expression.

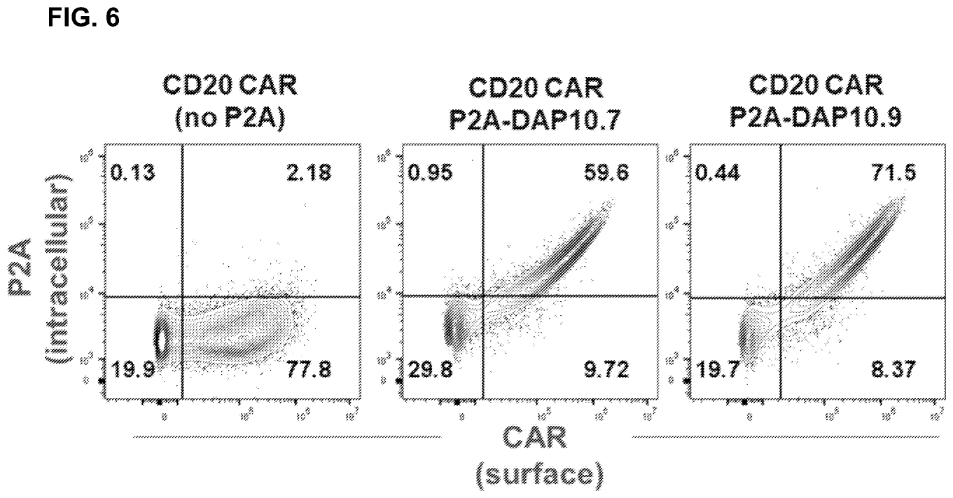

Vδ1 T cells modified with three different CD20 CAR constructs, two of which contain P2A and one that does not, were thawed and counted by Annexin-Dapi on the Novocyte (Agilent). Approximately 5×10⁵ cells were stained with cell viability dye Zombie Aqua (Biolegend). The cells were surface stained for Vδ1 (Biolegend) and anti-CAR idiotype antibody (Adicet Bio) and fixed with Fixation Buffer (Biolegend). The cells were then permeabilized with Permeabilization Buffer (BD Biosciences). After permeabilization, the cells were stained with anti-P2A antibody (Novus Biosciences) and acquired on the Novocyte (Agilent). The P2A positive population co-stained with CAR surface staining for samples CD20 CAR-P2A-DAP10.7 and CD20 CAR-P2A-DAP10.9 (FIG. 6). The results suggest intracellular staining of P2A can be used for CAR detection.

### Example 4. Gene Editing of γδ T Cells

This Example demonstrates the use of a CRISPR/Cas9 system to generate a CISH knockout in γδ T cells.

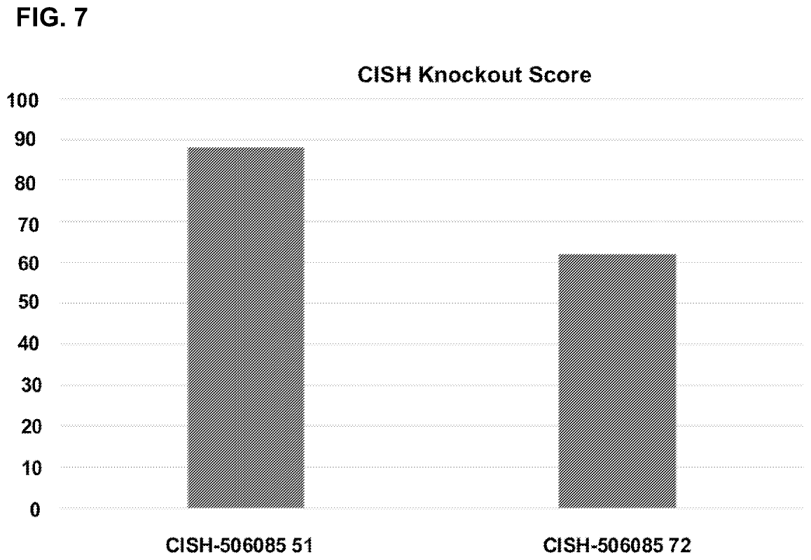

To knockout the CISH gene in Vδ1 T cells, a ribonucleoprotein (RNP) complex comprised of CRISPR/Cas9 (Integrated DNA Technologies) and CISH sgRNA's (Synthego Corporation) were combined with activated Vδ1 T cells from PBMC's (P3 Primary Cell 4D-Nucleofector X Kit L, Lonza). The RNP complex was delivered into Vδ1 T cells by electroporation (Lonza 4D-Nucleofector). Post electroporation, the cells were transferred to T flasks (Corning) containing X-Vivo15 (Lonza) for cell recovery and expansion in a 37 °C CO₂ incubator (ThermoFisher). To evaluate CISH KO efficiency, cell pellets were collected following αβ T cell depletion (StemCell Technologies) on day 7 post gene edit and genomic DNA was extracted using the NucleoSpin Tissue kit (MACHEREY-NAGEL). The edited region of the CISH gene was amplified by PCR and Sanger sequenced (Sequetech). Gene knockout efficiency was determined using the ICE Analysis tool (Synthego). High efficiency knockout of the CISH gene was observed from Vδ1 T cells (FIG. 7).

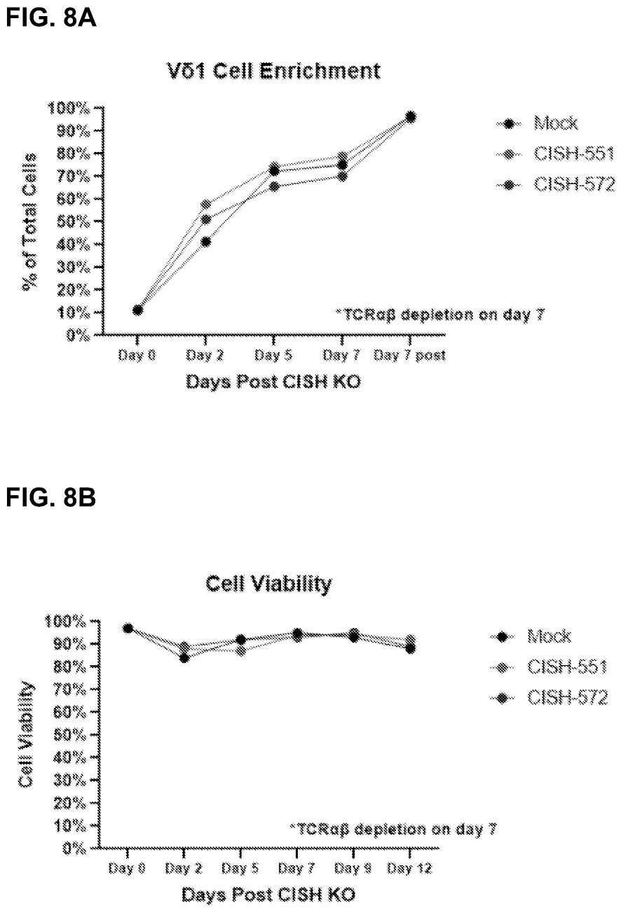

Post CISH gene knockout, Vδ1 T cell enrichment in PBMCs was monitored over time by flow cytometry (anti-Vδ1, BioLegend). CISH gene knockout did not affect Vδ1 T cell enrichment as compared to unedited Mock control cells. Similar purity of CISH gene edited Vδ1 T cells was achieved post TCRαβ depletion (StemCell Technologies) as compared to Mock control cells (FIG. 8A).

Cell viability of Vδ1 T cell enriched PBMCs was monitored post CISH gene knockout (Countess II, ThermoFisher). CISH KO cells maintained high cell viability similarly to unedited Mock control cells (FIG. 8B).

### Example 5. Identification of CAR Expression

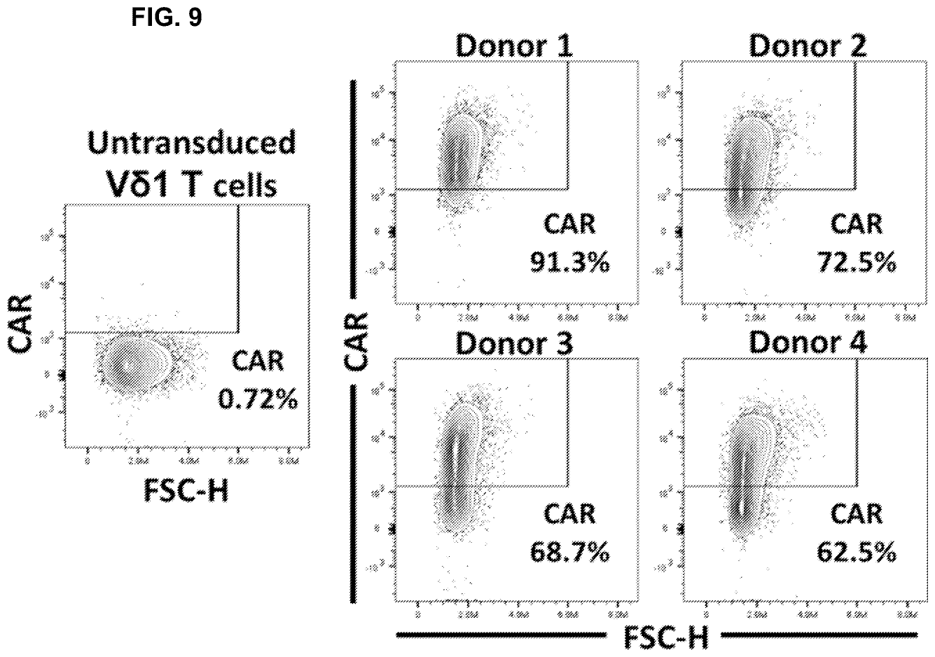

ADI-270 cells (cells expressing PL1179, SEQ ID NO: 57) were thawed and counted by Annexin-Dapi on the Novocyte (Agilent). Approximately 2×10⁵ cells were stained with cell viability dye Zombie Aqua (Biolegend). The cells were fixed with Fixation Buffer (Biolegend). The cells were then permeabilized with Permeabilization Buffer (BD Biosciences). After permeabilization, the cells were stained with anti-P2A antibody (Novus Biosciences) and acquired on the Novocyte. The P2A positive population represent the transduction efficiency of the CAR. The results are shown in FIG. 9, which suggest that intracellular staining of P2A can be used for CAR detection of ADI-270.

### Example 6. In Vitro Activity of ADI-270

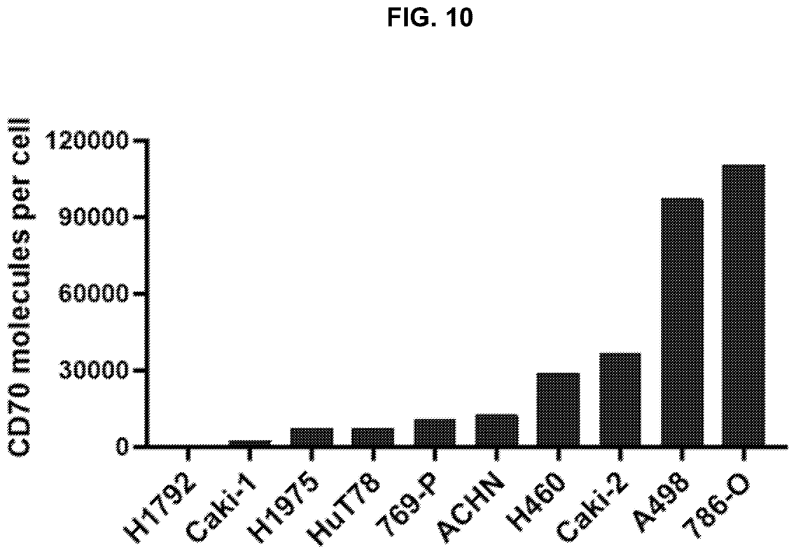

Target cell lines Caki-1, Caki-2, 769-P, ACHN, A498, 786-O, H1792, H1975, H460, and HuT78 (ATCC) were stained with anti-CD70 antibody conjugated to PE (Biolegend) or isotype control conjugated to PE (R&D Systems). Cells were analyzed on the Novocyte. The number of CD70 molecules per cell was quantified using the flow cytometric BD Quantibrite™ Bead Kit (BD Biosciences) (FIG. 10). Several tumor cell lines that expressed a wide range of CD70 levels were identified for subsequent in vitro functional evaluation for ADI-270.

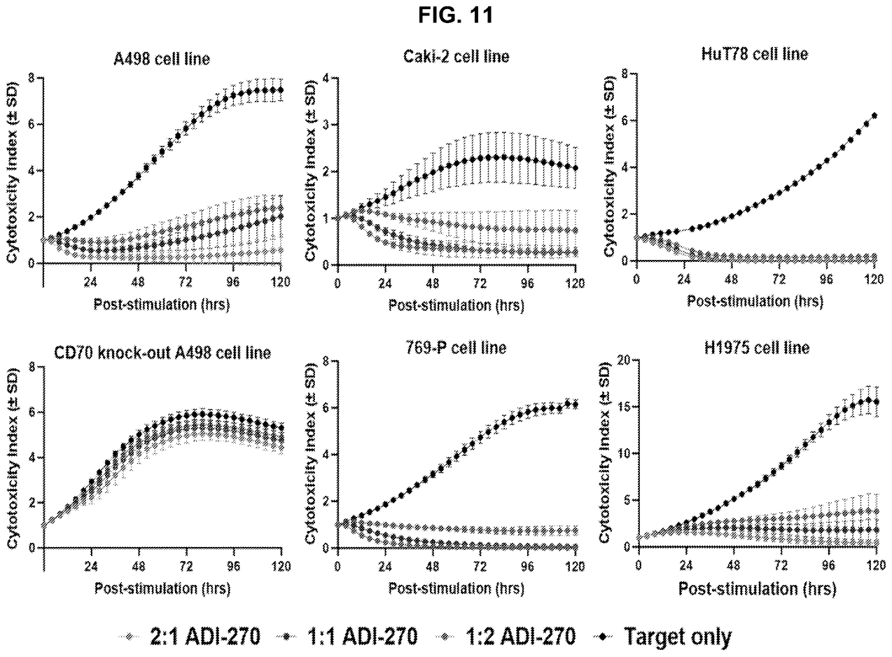

For the long-term cytotoxicity assay, ADI-270 was co-cultured with target cell lines at an effector to target ratio (E:T) of 2:1, 1:1, and 1:2 for 120 hours. Target cells were modified to express Nuc-NIR by transducing them with Incucyte® Nuclight NIR lentivirus (Sartorius). Viable Nuc-NIR expressing target cells were quantified every 4 hours by the Incucyte® SX5 (Sartorius). Cytotoxicity index was calculated by dividing the total NIR object area of each time point by the value of time 0 hours. Increased tumor cell killing is represented by a lower Cytotoxicity Index. As shown in FIG. 11, ADI-270 showed potent cytotoxic activity when co-cultured against multiple RCC cell lines (A498, 769-P, Caki-2) expressing a range of CD70 levels, and against CD70-positive NSCLC (H1945) and T cell lymphoma (HuT78) cell lines. No cytotoxicity against the CD70 knock-out A498 cell line was observed at the E:T ratios tested, suggesting the cytolytic activity of ADI-270 is specific and dependent on the expression of CD70.

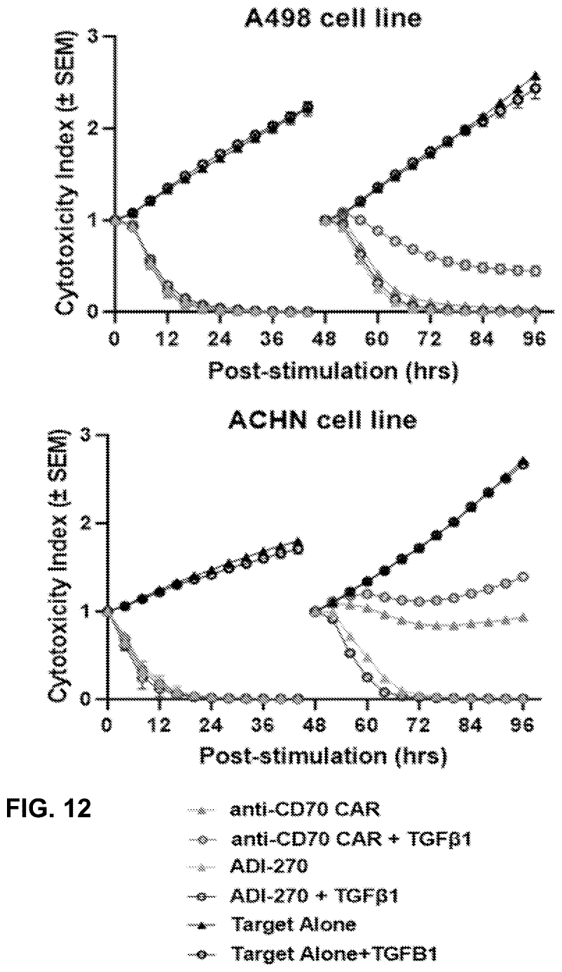

ADI-270 is equipped with dnTGFβRII armoring to mitigate the immunosuppressive effects of TGFβ1 that can exist within the tumor microenvironment (TME). To demonstrate the functionality of the dnTGFβRII expressed by ADI-270, cytolytic activity against CD70-positive cell lines was compared to anti-CD70 CAR Vδ1 γδ T cells (no dnTGFβRII) (PL1107, SEQ ID NO: 53) in the absence and presence of exogenously added TGFβ1 (R&D Systems) using an in vitro repetitive stimulation assay. A498- and ACHN-Nuc NIR fluorescently labeled RCC tumor cell lines and effector cells were added into a 96-well plate at an ET ratio of 5:1. TGFβ1 was added to the respective co-culture conditions at a final concentration of 20 ng/ml. The first stimulation was monitored for 2 days after which fresh target cells were plated in another 96-well plate. The effector cells from the first stimulation were then transferred into the plate with fresh target cells. The second stimulation was monitored for an additional 2 days. The co-cultures were monitored every 4 hours using Incucyte® SX5. The Cytotoxicity Index was calculated based on the remaining viable target tumor cells by dividing the total NIR object area of each time point by the value at time 0 for each stimulation. The results are shown in FIG. 12. In the presence of TGFβ1, ADI-270 maintained cytolytic activity against A498 and ACHN when compared to the no TGFβ1 condition after the second stimulation with tumor cells. The anti-CD70 CAR Vδ1 γδ T cells in the presence of TGFβ1 were associated with the lowest tumor control after the second stimulation due to the lack of armoring to mitigate the inhibitory effect by TGFβ1. The data suggests ADI-270 maintains its potent in vitro cytotoxicity against RCC cell lines in the presence of TGFβ1, demonstrating the functionality of dnTGFβRII armoring in Vδ1 γδ T cells.

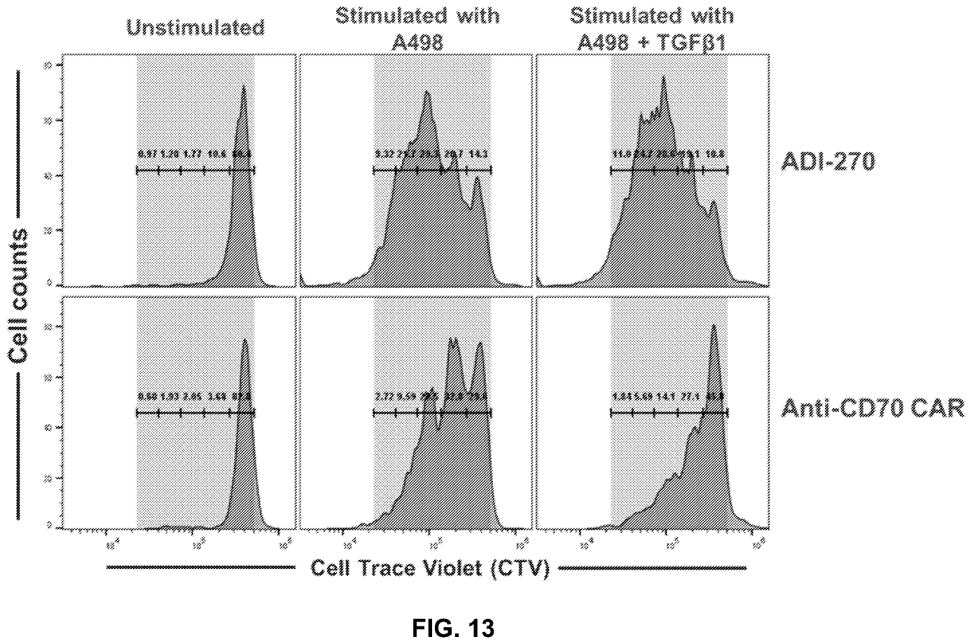

TGFβ1 is known to directly inhibit T cell proliferation. To further assess the functionality of dnTGFβR2 armoring, in vitro proliferation of ADI-270 was evaluated in the absence and presence of exogenously added TGFβ1 (R&D Systems). ADI-270 and anti-CD70 CAR Vδ1 γδ T cells (effector cells) were labeled with Cell Trace Violet (CTV) (ThermoFisher), to measure their proliferative capacity. The CTV-labeled effector cells were cocultured with A498 target cells at an E:T ratio of 1:2 for 7 days. The coculture was harvested, and the effector cells were evaluated for proliferation using flow cytometry (Novocyte) by determining the % of cell divisions with decreased CTV fluorescent intensity using FlowJo™ Proliferation Modeling software (FlowJo). The results are shown in FIG. 13. In the presence of TGFβ1, ADI-270 maintained in vitro proliferation after stimulation with A498 tumor cells when compared to without TGFβ1 added based on the similar % of cell divisions detected. In contrast, the anti-CD70 CAR Vδ1 γδ T cells had reduced % cell divisions detected when stimulated with A498 cells in the presence of TGFβ1 compared to the no TGFβ1 condition. The results indicate that dnTGFβRII armoring reduces the effects of TGFβ1 to inhibit proliferation of Vδ1 γδ T cells. The data is a representative of 4 different donors. The numbers represent the % of cells within each gated cell division.

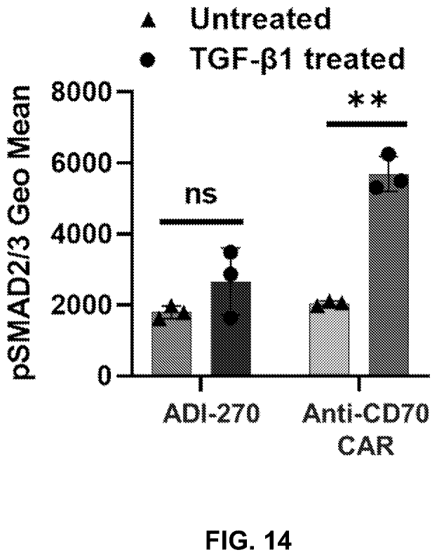

The binding of TGFβ1 to the heteromeric complex of TGFβ type II, and type I receptors induce a signaling cascade that phosphorylates SMAD2 and SMAD3 (pSMAD2/3). To investigate if dnTGFβRII armoring in ADI-270 can reduce the phosphorylation of SMAD2/3, ADI-270 and anti-CD70 CAR Vδ1 γδ T cells were serum starved for 2 hours and cultured in the presence and absence of exogenous TGFβ1 (R&D Systems) for 15 minutes. The cells were then fixed, permeabilized, and stained for anti-pSMAD2/3 and analyzed using flow cytometry (Novocyte). The results are shown in FIG. 14. After exposure to TGFβ1, ADI-270 was associated with lower levels of pSMAD2/3 compared to anti-CD70 CAR Vδ1 γδ T cells which further supports the functionality of the dnTGFβRII. The modest increase in pSMAD2/3 with ADI-270 compared to the no TGFβ1 condition is likely attributed to the presence of untransduced CAR Vδ1 γδ T cells within the total population that are not armored with dnTGFβR2. Data shown is the average Geo Mean from 3 different donors. Statistical analysis was performed using paired t-test ** p<0.005.

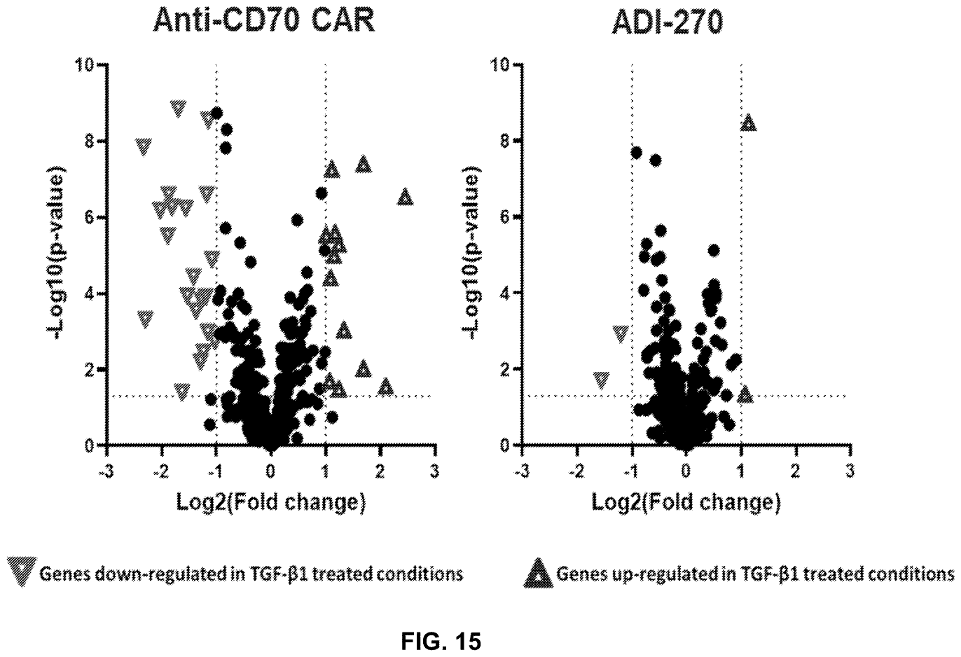

To determine if dnTGFβRII armoring of ADI-270 can minimize changes in transcripts levels associated with T cell effector functions, ADI-270 and anti-CD70 CAR Vδ1 γδ T cells were activated with rhuCD70 (AcroBio) for 24 hrs in the presence and absence of TGF-β1 (R&D Systems). RNA was extracted from the cells using the RNeasy Plus Kits (Qiagen). The nCounter® CAR-T Characterization Panel (Nanostring) and nCounter® SPRINT Profiler (Nanostring) were used to quantitate T cell-related transcripts from the samples. The nCounter® Advanced Analysis Software (Nanostring) was used to identify differentially expressed genes (DEGs). The results are shown in FIG. 15. Volcano plots represent DEGs from ADI-270 vs anti-CD70 CAR Vδ1 Y& T cells (not armored) activated with rhuCD70 (5 μg/mL) in the presence or absence of TGF-β1 (20 ng/mL) for 24 hrs from 2 different donors. Blue (up pointing) triangles represent DEGs upregulated in TGF-β1 treated vs untreated conditions. Red (down pointing) triangles represent DEGs downregulated in TGF-β1 treated vs untreated conditions. Stimulated anti-CD70 CAR Vδ1 γδ T cells were associated with increased transcript changes when compared between +/−TGF-1 conditions. Minimal transcript changes were observed from stimulated ADI-270 between +/−TGF-β1 conditions further supporting dnTGFβRII armoring is minimizing TGF-β1-induced transcript changes in Vδ1 γδ T cells.

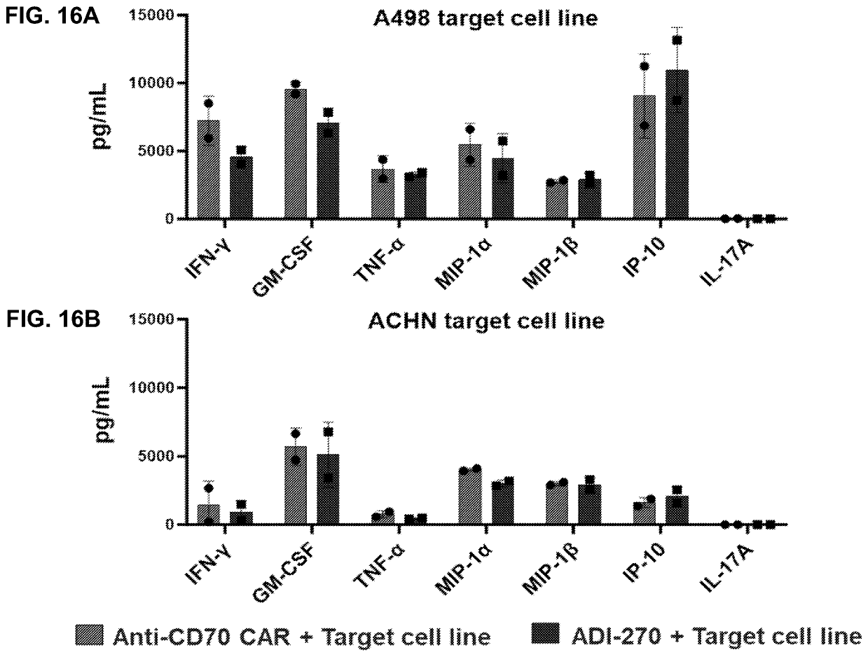

ADI-270 has multiple effector functions, such as the production of soluble mediators, and recognition and killing of CD70+ tumor cells, that contributes to effective anti-tumor activity. To investigate the cytokine and chemokine production of ADI-270 and anti-CD70 CAR Vδ1 γδ T cells, a coculture with A498 and ACHN target cell lines at an E:T ratio of 1:1 was performed, and supernatants were collected after 24 hours post-stimulation. Supernatants were analyzed using the Milliplex Immunology Multiplex Assay Panel on the Luminex® FLEXMAP 3D instrument. Analysis of the supernatants revealed that IFN-γ, GM-CSF, TNF-α, MIP-1α, MIP-1β, IL-17A, and IL-10 were measured at similar levels for both ADI-270 and anti-CD70 CAR Vδ1 γδ T cells after stimulation with A498 and ACHN cell lines. The results are shown in FIGS. 16A-16B. The data suggests that the dnTGFβRII armoring in ADI-270 does not alter the cytokine and chemokine release profile when compared to anti-CD70 CAR Vδ1 γδ T cells. Minimal cytokine and chemokine secretion was seen with ADI-270 and target alone. Data shown is the mean concentration (pg/mL) of cytokine and chemokines derived from two different donors.

### Example 7. In Vivo Control of Human Tumor Xenografts in NSG Mice with ADI-270 Treatment

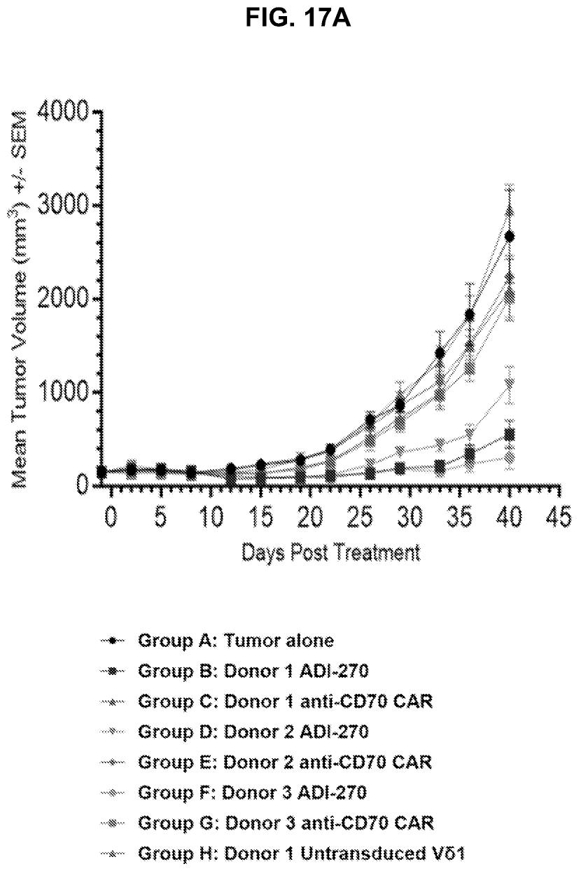

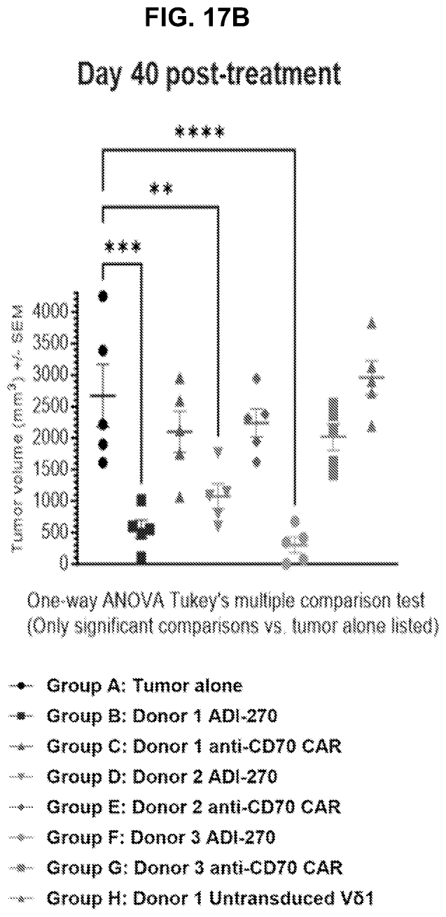

ADI-270 was designed with a dnTGFβRII armor against the immunosuppressive factor TGF-β1. Research grade ADI-270 and anti-CD70 CAR Vδ1 γδ T cells (no dnTGFβRII) were manufactured from three separate donors. The activity of ADI-270 with or without armoring was then tested in a CD70-expressing human renal cell carcinoma (RCC) cell line A498 implanted subcutaneously in female NSG mice (Charles River) (n=5 per group). Mice were randomized into study cohorts when tumor volumes were approximately 150 mm³. 5E6 ADI-270, 5E6 anti-CD70 CAR Vδ1 γδ T cells, and 5E6 untransduced γδ1 T cells were administered on Day 0 as a single bolus dose. Human IL-2 supplementation was given three times a week for the duration of the study. Tumor volume and mouse weight were monitored over the duration of the experiment. The results are shown in FIGS. 17A-17B. (FIG. 17A) Mean tumor volumes (MTV) with standard error bars for each group are shown over the duration of the study until the endpoint was reached on Day 40, when mean tumor volumes in the tumor alone cohort reached approximately 2000 mm³. (FIG. 17B) Statistical analysis of Day 40 tumor volume data (end-of-study) was performed using one-way ANOVA, with Tukey's multiple comparison test. ADI-270 caused no adverse health effects and showed significant tumor control compared to the untreated controls or mice treated with untransduced γδ T cells. Furthermore, ADI-270 demonstrated functional advantages with significant enhancement of efficacy when compared to unarmored anti-CD70 CAR Vδ1 γδ T cells.

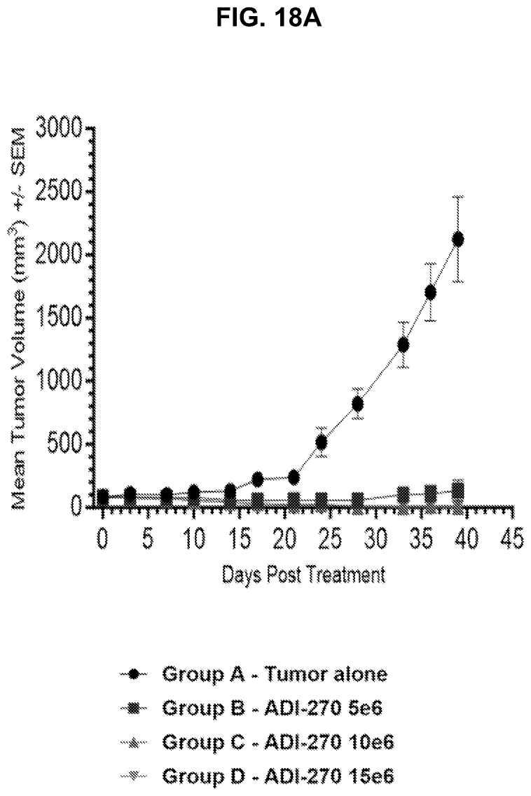

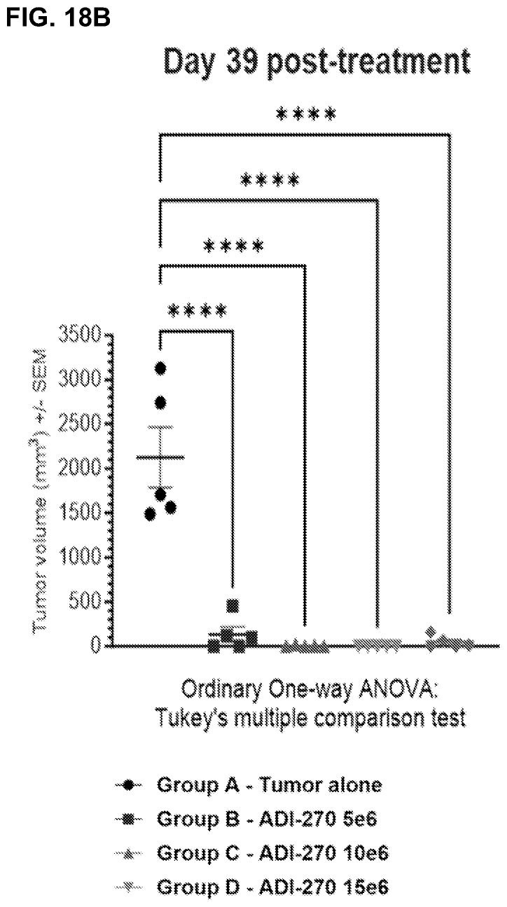

Technical grade ADI-270 material was evaluated in A498 human RCC subcutaneous xenograft tumor model at three dose levels (5e6, 10e6 and 15e6 CAR+ viable cells). Human renal cell carcinoma A498 subcutaneous xenografts were established by inoculation of 4E6 cells/mouse on the right hind flank of female NSG mice. Mice were randomized into study cohorts when tumor volumes were approximately 150 mm³ (N=5 mice per group). ADI-270 was administered on Day 0 as a single bolus dose. Human IL-2 supplementation was given three times a week for the duration of the study. Tumor volume and mouse weight were monitored over the duration of the experiment. The results are shown in FIGS. 18A-18B. (FIG. 18A) Average tumor volumes for the duration of the study and (FIG. 18B) statistical comparison (Tukey's multiple comparison test) between treatment groups and the vehicle control group at the end of the study (Day 39) when mean tumor volumes in the tumor alone cohort reached approximately 2000 mm³ was performed. **** represents a p value of ≤0.0001. Technical grade ADI-270 caused no adverse health effects and showed similar potency as research grade material previously tested in this model. Higher dose levels of ADI-270 (10e6 and 15e6) led to complete eradication of tumors in this RCC solid tumor model.

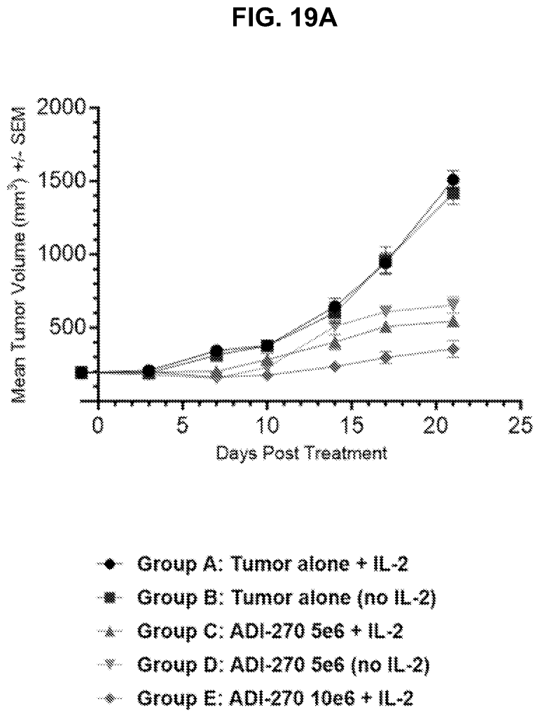

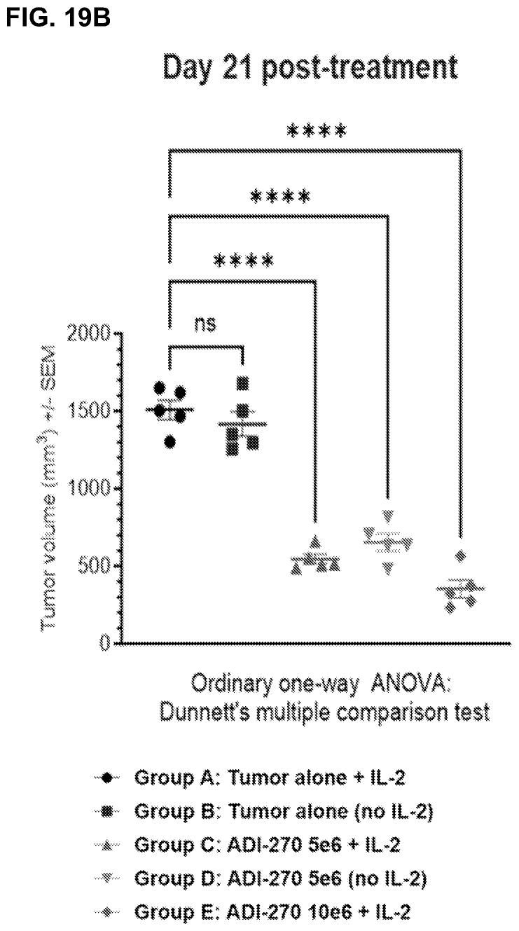

The activity of ADI-270 was assessed against a different indication with lower levels of CD70 expression. HuT78, a CD70-low expressing human T cell lymphoma cell line, was implanted subcutaneously by inoculation of 2E6 HuT78 cells on the right hind flank of female NSG mice. Mice were randomized into study cohorts when tumor volumes reached approximately 200 mm³ (N=5 per group). ADI-270 was administered on Day 0 as a single bolus dose. Human IL-2 supplementation was given to appropriate groups three times a week for the duration of the study. The results are shown in FIGS. 19A-19B. (FIG. 19A) Mean tumor volumes (MTV) with standard error bars for each group are shown over the duration of the study until the endpoint was reached on Day 21, when mean tumor volumes in the tumor alone cohort reached approximately 2000 mm³. (FIG. 19B) Statistical analysis of Day 21 tumor volume data (end-of-study) was performed using one-way ANOVA with Dunnett's multiple comparison test. **** represents a p value of ≤0.0001. ADI-270 showed significant control with and without IL-2 supplementation in a dose-dependent fashion.

### Example 8. Ex Vivo Tissue Analysis from ADI-270 Treated NSG Mice Implanted with Human Tumor Xenografts

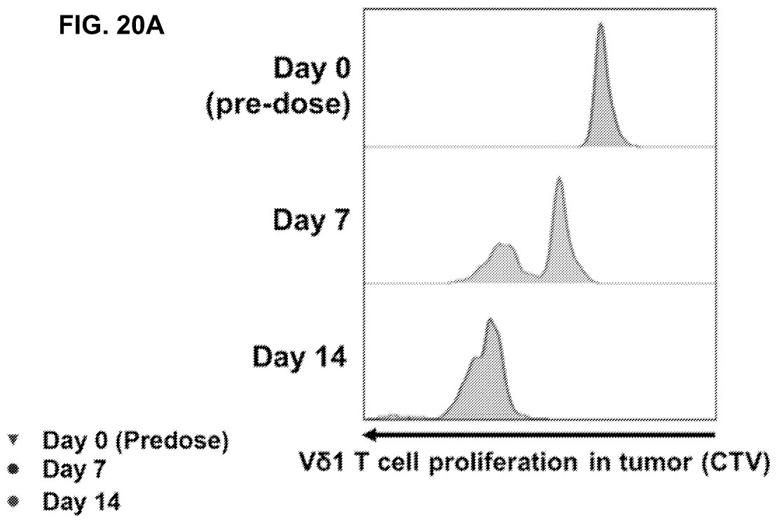

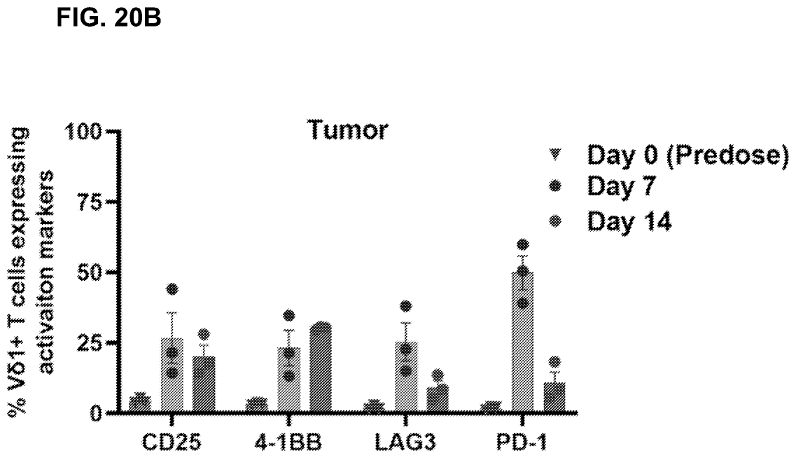

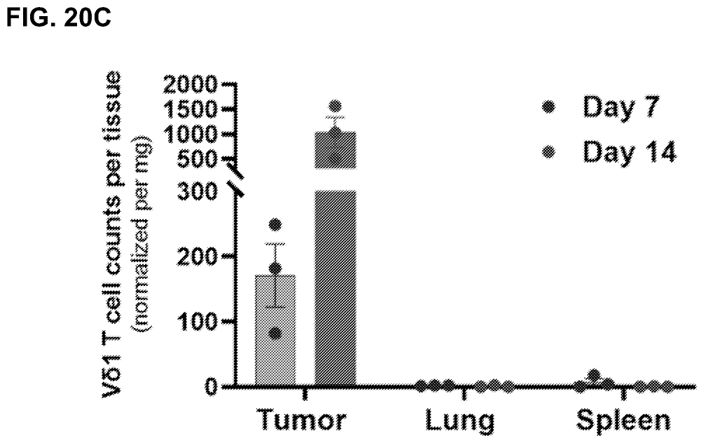

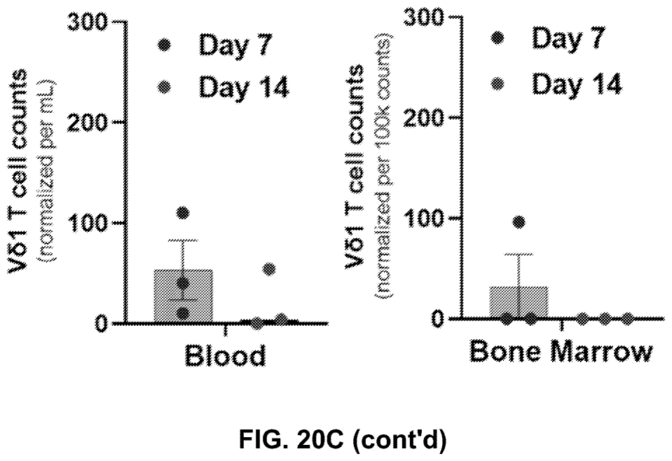

Based on the robust in vivo tumor control without incidence of xenogeneic GvHD in A498 tumor-bearing NSG mice, the proliferation and activation of ADI-270 specifically within the tumor, with little to no accumulation in normal tissues was confirmed. Tissues from A498-bearing NSG mice receiving a single IV dose of Cell Trace Violet (CTV) (ThermoFisher) labeled ADI-270 were harvested at day 7 and 14. The results are shown in FIGS. 20A-20C. (FIG. 20A) CTV labeled ADI-270 shows increased cell proliferation in tumor as indicated by the dilution of the CTV dye (histogram plots) compared to day 0 control. (FIG. 20B) Harvested ADI-270 were stained for activation-associated markers and (FIG. 20C) ADI-270 was seen to specifically home to the tumor when compared to lung, spleen, blood, and bone marrow at day 7 and 14.

### Example 9. Host Vs GRAFT (HvG) Armoring of ADI-270

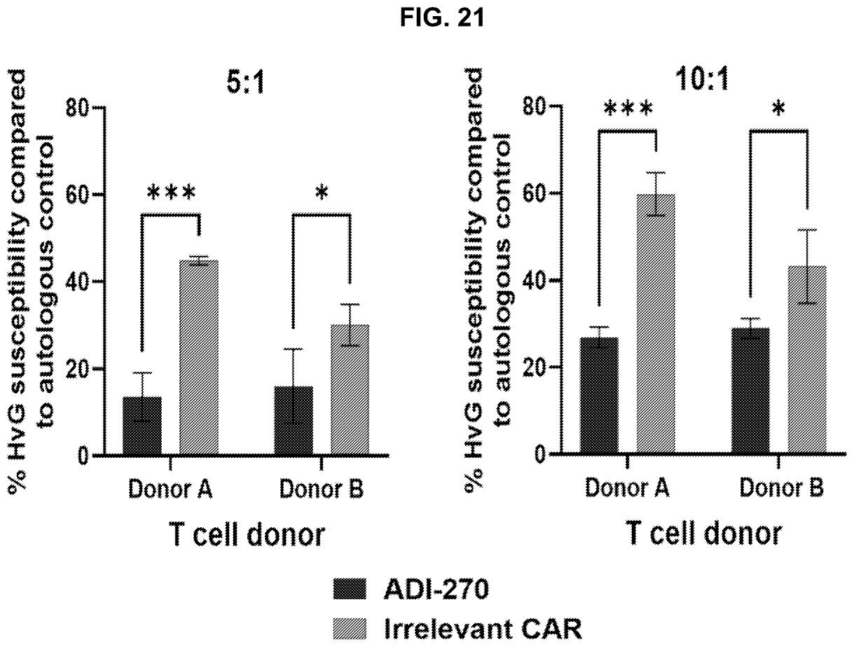

ADI-270 and irrelevant CAR (targeting CD20) Vδ1 γδ T cells (target cells) were co-cultured with T cells (effectors) isolated from autologous and allogeneic (Donor A and B) PBMC donors using the EasySep™ Human T Cell Enrichment Kit (StemCell Technologies) at a fixed ratio E:T ratio of 5:1 and 10:1 with IL-2 supplementation. Half of the culture media (10% FBS RPMI-1640) was replaced every 2-3 days with fresh IL-2. On day 5, the cells were harvested and analyzed by flow cytometry (Novocyte) to quantitate the absolute counts of ADI-270 and irrelevant CAR Vδ1 γδ T cells from the co-culture with autologous and allogeneic T cells. The % HvG susceptibility was determined by comparing the absolute counts of ADI-270 and irrelevant CAR Vδ1 γδ T cells effectors cells of the T cell autologous co-cultures (control for minimal cell killing) to the T cell allogeneic co-cultures. The results are shown in FIG. 21. Reduced HvG susceptibility of ADI-270 was observed compared to Vδ1 T cells expressing an irrelevant CAR suggesting ADI-270 potentially targets CD70+ activated alloreactive host T cells and can limit HvG Vδ1 T cell rejection to aid in persistence. Statistical comparison between ADI-270 and irrelevant CAR groups was determined using 2-way ANOVA. The results suggest that using a dual CAR binding to CD70 and another tumor-associated antigen, or co-administering a CAR binding to CD70 and a CAR binding to another tumor associated antigen may have reduced alloreactivity compared to using the CAR binding to another tumor-associated antigen alone.

### Example 10: Enhancement of CAR Vδ1 T Cells Effector Function Using Membrane Bound IL-12 (mbIL-12)

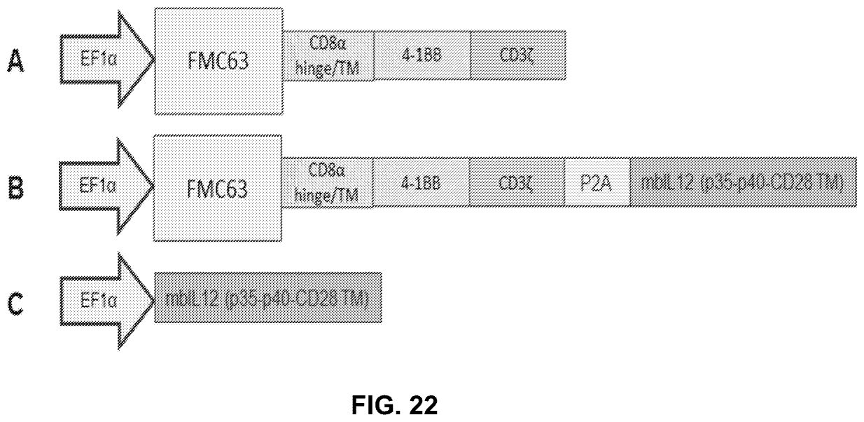

CAR vector, consisting of the anti-CD19 (clone FMC63) scFv (as described in Kochendefer J N et al., Adoptive transfer of syngeneic T cells transduced with a chimeric antigen receptor that recognizes murine CD19 can eradicate lymphoma and normal B cells, Blood. 2010 Nov. 11; 116(19):3875-86), CD8a hinge and transmembrane domains, and 4-1BB and CD3ζ signaling domains followed by a self-cleaving P2A sequence that is used to separate the mbIL-12 molecule (as described in Lee et al., Antigen-dependent IL-12 signaling in CAR T cells promotes regional to systemic disease targeting, bioRxiv. 2023 Jan. 7; 2023.01.06.522784), was constructed by cloning a DNA fragment containing all CAR domains (Integrated DNA Technologies) into a self-inactivating (SIN) Moloney Murine Leukemia Virus (MMLV) gamma-retroviral plasmid. The constructs design is shown in FIG. 22 (construct A) CAR (FMC63) construct, (construct B) CAR-mbIL-12 construct, and (construct C) mbIL-12 construct.

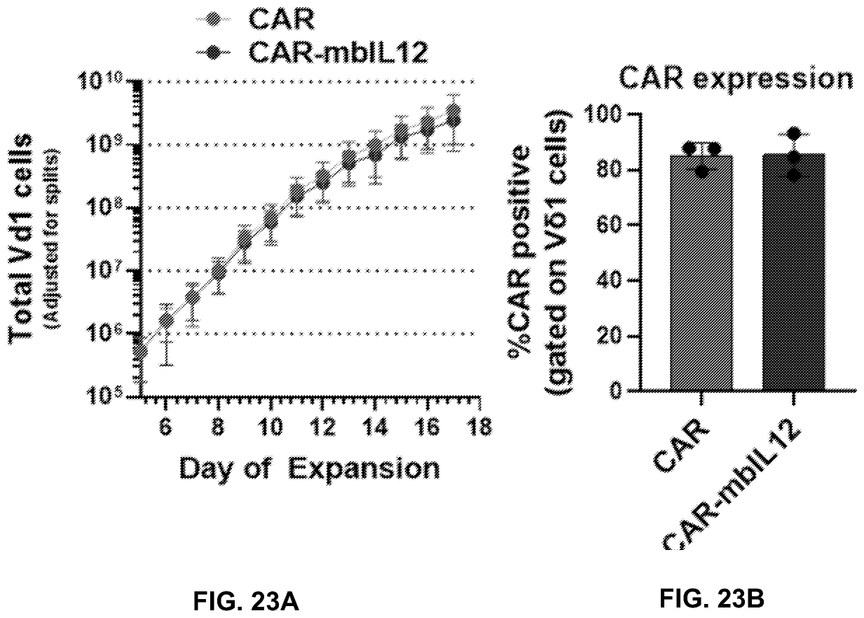

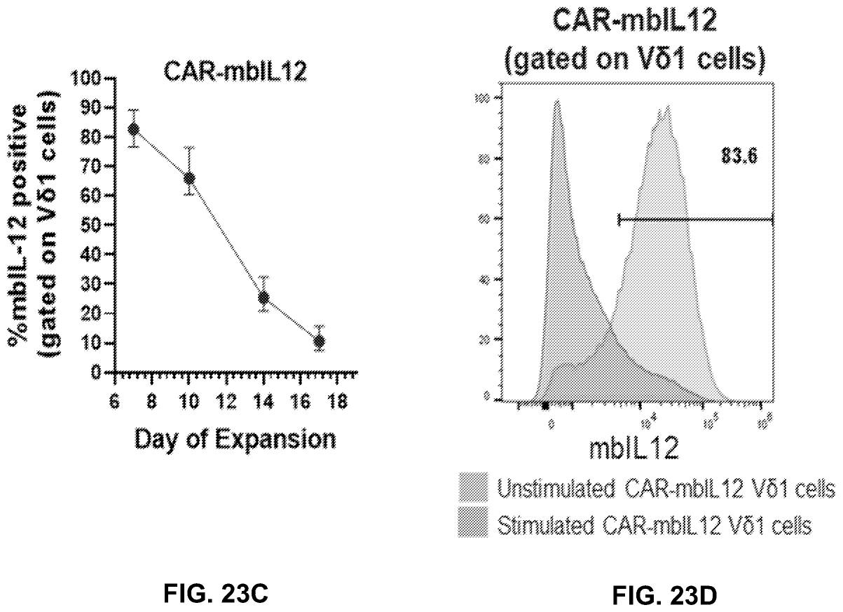

Healthy donor peripheral blood mononuclear cells (PBMCs) were used to activate, expand and engineer Vδ1 γδ T cells to express the CD19 CAR-mbIL-12 transgene. The results are shown in FIGS. 23A-23D. (FIG. 23A) Vδ1 γδ T cells (post-transduction) were sampled at different time points over the course of the expansion to determine cell counts. The total Vδ1 γδ T cell counts did not differ between CAR and CAR-mbIL-12, suggesting mbIL-12 has no negative impact on the expansion of Vδ1 yd T cells. Cells were harvested, stained for CAR and mbIL-12 using anti-FMC63 antibody conjugated to PE (Acrobio Systems, Newark, DE) and anti-IL12 conjugated to APC (Miltenyi Biotec) respectively, and acquired on the Novocyte flow cytometer (Agilent Technologies) to determine the percentage of CAR and mbIL-12 positive Vδ1 γδ T cells. (FIG. 23B) No difference in % CAR was observed between Vδ1 γδ T cells expressing CAR and CAR-mbIL-12 at the end of the process to further support mbIL-12 did not affect CAR surface expression. (FIG. 23C) Over the course of the expansion, the % mbIL-12 expressed on the cell surface of Vδ1 γδ T cells decreased over time. (FIG. 23D) CAR-mbIL-12 Vδ1 γδ T cells were co-cultured with and without CD19+ Raji cells for 18 hrs. After stimulation with target cells, mbIL-12 increased on the cell surface of the Vδ1 γδ T cells compared to unstimulated conditions suggesting the surface expression of mbIL-12 is dependent on CAR activation.

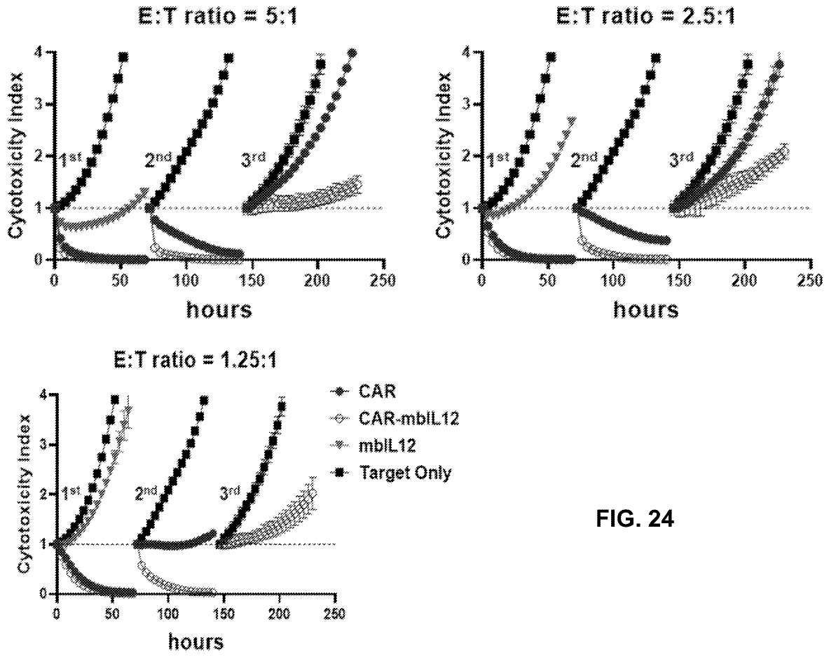

To demonstrate the functionality of CAR-mbIL-12, cytolytic activity against CD19-positive cell lines using CAR, CAR-mbIL-12, and mbIL-12 transduced Vδ1 γδ T cells was evaluated in an in vitro repetitive stimulation assay. Raji-Nuc NIR fluorescently labeled cell line and effector cells were added into a 96-well plate at E:T ratios of 5:1, 2.5:1, and 1.25:1. The first stimulation was monitored for 3 days after which fresh target cells were plated in another 96-well plate. The effector cells from the first stimulation were then transferred into the plate with fresh target cells. The second stimulation was monitored for an additional 3 days, and the same procedure was performed for a third stimulation. The co-cultures were monitored every 4 hours using Incucyte® SX5 (Satorius). The Cytotoxicity Index was calculated based on the remaining viable target tumor cells by dividing the total NIR object area of each time point by the value at time 0 for each stimulation. The lower the cytotoxicity index, the better the cytotoxic potential. After the second and third stimulation, the CAR-mbIL-12 Vδ1 γδ T cells were associated with greater tumor cell killing compared to CAR and mbIL12 Vδ1 γδ T cells across the E:T ratios tested. Co-culture conditions with a Cytotoxicity Index>1 (indication of incomplete tumor control), were not analyzed in the subsequent stimulation. The results are shown in FIG. 24 and demonstrate the functionality of the CAR-mbIL-12 expressed by Vδ1 γδ T cells to sustain cytolytic killing of tumor cells.

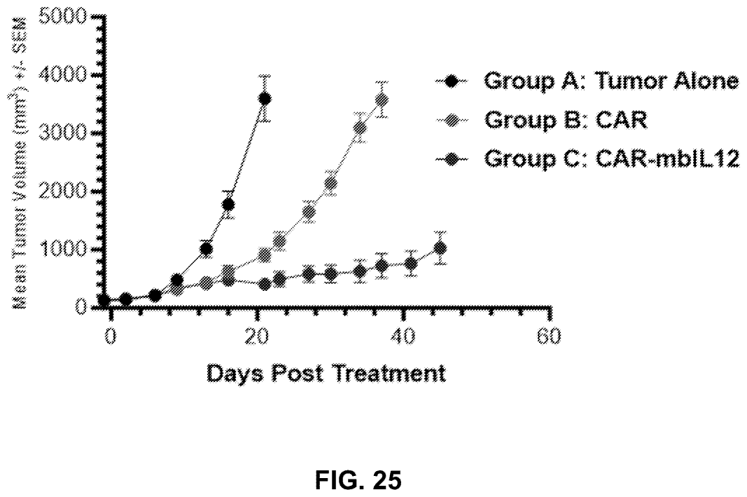

CAR-mbIL-12 Vδ1 γδ T cells were evaluated in Raji cell subcutaneous xenograft tumor model at a suboptimal dose of 2.5e6 CAR+ viable cells. Raji cell subcutaneous xenografts were established by inoculation of 1E6 cells/mouse on the right hind flank of female NSG mice. Mice were randomized into study cohorts when tumor volumes were approximately 200 mm³ (N=5 mice per group). V8l yd T cells expressing CAR and CAR-mbIL-12 were administered on Day 0 as a single bolus dose. Human IL-2 supplementation was given three times a week for the duration of the study. Tumor volume and mouse weight were monitored over the duration of the experiment. The results are shown in FIG. 25. The CAR-mbIL-12 Vδ1 γδ T cells were associated with a substantially greater inhibition of tumor cell growth compared to the CAR Vδ1 γδ T cell group at the suboptimal treatment dose. The data supports the enhanced functionality of Vδ1 γδ T cells expressing CAR-mbIL-12.

### Example 11: CISH KO Enhances the Function of Vδ1 T Cells

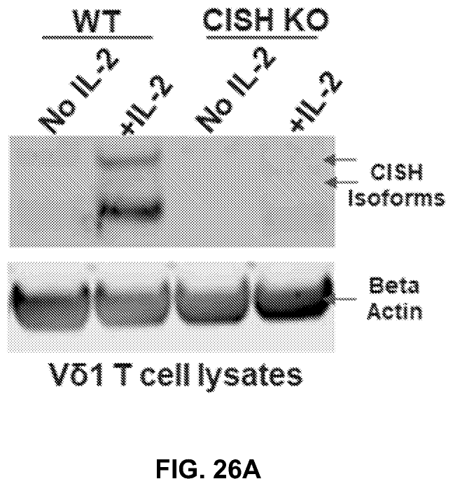

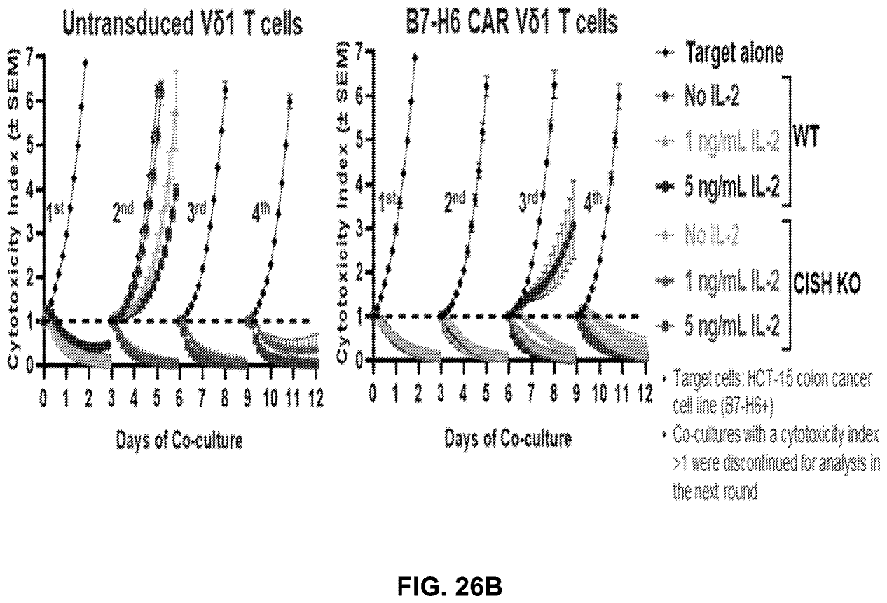

Healthy donor peripheral blood mononuclear cells (PBMCs) were used to activate, expand, CRISPR-gene edit, and engineer Vδ1 γδ T cells to express a CAR. To knockout the CISH gene in Vδ1 T cells, a ribonucleoprotein (RNP) complex comprising CRISPR-Cas9 (Integrated DNA Technologies) or -MAD7 (Aldevron) and CISH sgRNA were combined with activated Vδ1 T cells from PBMCs (P3 Primary Cell 4D-Nucleofector X Kit L, Lonza). The RNP complex was delivered into Vδ1 T cells by electroporation (Lonza 4D-Nucleofector). Post electroporation, the cells were transferred to T flasks (Corning) containing X-Vivo15 (Lonza) for cell recovery prior to transduction with gammaretroviral vector encoding the CAR and then were subsequently expanded. To evaluate CISH KO efficiency, cell pellets were collected, and genomic DNA was extracted using the NucleoSpin Tissue kit (MACHEREY-NAGEL). The edited region of the CISH gene was amplified by PCR and Sanger sequenced (Sequetech). Gene knockout efficiency was determined using the ICE Analysis tool (Synthego). To evaluate the KO of CISH protein in CAR Vδ1 T cells, WT (unedited) and CISH KO CAR Vδ1 T cells were incubated with and without IL-2 (Peprotech) for 5 hours. Cell lysates were generated for western blot detection of CISH protein using anti-CISH Rabbit mAb (Cell Signaling). An IRDye 800CW goat anti-Rabbit IgG secondary antibody (LI-COR) was used for detection of the primary antibody and the presence of the IRDye was imaged with the Odyssey imaging system (LI-COR). The results are shown in FIGS. 26A-26B. (FIG. 26A) WT CAR Vδ1 T cells showed an increase in levels of CISH protein when exposed to IL-2. In contrast, CISH KO CAR Vδ1 T cells had substantially reduced levels of CISH protein in the presence of IL-2 supporting the KO of CISH. (FIG. 26B) To demonstrate enhanced function of CISH KO CAR-transduced and untransduced Vδ1 T cells, the cytolytic activity against the B7-H6+ HCT-15 cell line (ATCC) was evaluated using an in vitro repetitive stimulation assay. HCT-15 NIR fluorescently labeled cell line and effector cells were added into a 96-well plate at an ET ratio of 5:1 with or without different concentrations of IL-2. Each stimulation was monitored for 3 days after which fresh target cells were plated in another 96-well plate. The effector cells from each stimulation were then transferred into the plate with fresh target cells. The co-cultures were monitored every 4 hours using Incucyte® SX5 (Satorius). The Cytotoxicity Index was calculated based on the remaining viable target tumor cells by dividing the total NIR object area of each time point by the value at time 0 for each stimulation. The lower the cytotoxicity index, the better the cytotoxic potential. After the second stimulation, CISH KO untransduced Vδ1 T cells were associated with greater tumor cell killing compared to WT untransduced Vδ1 T cells only in the presence of IL-2. Without IL-2, CISH KO CAR Vδ1 T cells had superior tumor control compared to WT CAR Vδ1 T cells. The results suggest CISH KO enhances the in vitro cytotoxicity of Vδ1 T cells.

CAS9 CISH sgRNA:
(SEQ ID NO: 96)
TGTACAGCAGTGGCTGGTGG(AGG)
MAD7 CISH sgRNA:
(SEQ ID NO: 97)
(TTTA)GGTGTACAGCAGTGGCTGGTG

### Example 12: CBL-B KO Enhances the Function of Vδ1 T Cells

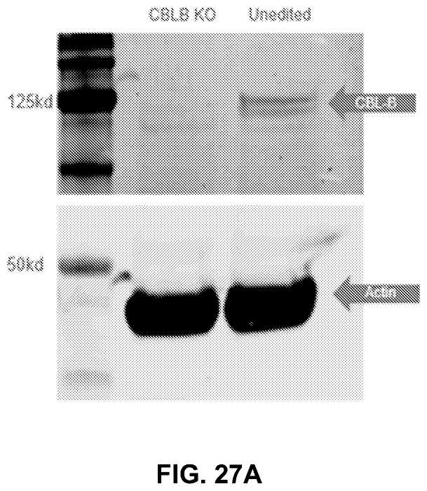

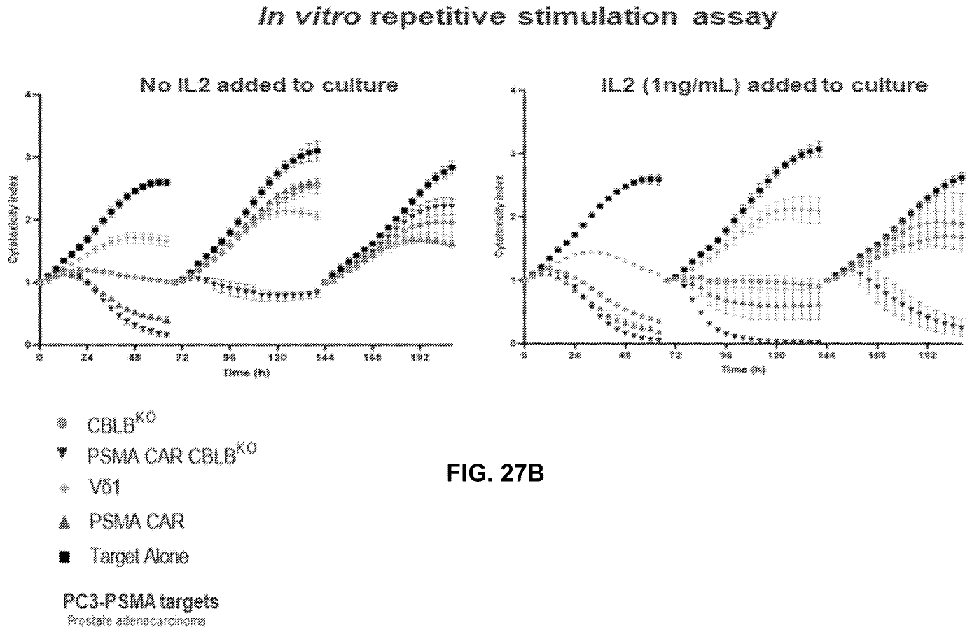

Healthy donor peripheral blood mononuclear cells (PBMCs) were used to activate, expand, CRISPR-gene edit, and engineer Vδ1 γδ T cells to express a CAR. To knockout the CBL-B gene in Vδ1 T cells, a ribonucleoprotein (RNP) complex comprising CRISPR-Cas9 (Integrated DNA Technologies) and CBL-B sgRNAs were combined with activated Vδ1 T cells from PBMCs (P3 Primary Cell 4D-Nucleofector X Kit L, Lonza). The RNP complex was delivered into Vδ1 T cells by electroporation (Lonza 4D-Nucleofector). Post electroporation, the cells were transferred to T flasks (Corning) containing X-Vivo15 (Lonza) for cell recovery prior to transduction with gammaretroviral vector encoding the CAR and then were subsequently expanded. The results are shown in FIGS. 27A-27B. (FIG. 27A) To determine CBL-B KO efficiency, genomic DNA was extracted from cell pellets using the NucleoSpin Tissue kit (MACHARY-NAGEL). The edited region was amplified by PCR using previously in-house optimized primers. The PCR reaction was run on an agarose gel, and the prominent band excised, and gel extracted using NucleoSpin Gel and PCR Cleanup kit (MACHARY-NAGEL). The extracted DNA was sequenced by Sanger sequencing (Sequetech). % Indels were determined using the ICE Analysis tool (Synthego). (FIG. 27B) To demonstrate enhanced function of CBL-B KO CAR-transduced and untransduced Vδ1 T cells, the cytolytic activity against the PSMA+ PC3 cell line was evaluated using an in vitro repetitive stimulation assay. PC3-PSMA NIR fluorescently labeled cell line and effector cells were added into a 96-well plate at an ET ratio of ~3:1 with or without IL-2. Each stimulation was monitored for 3 days after which fresh target cells were plated in another 96-well plate. The effector cells from each stimulation were then transferred into the plate with fresh target cells. The co-cultures were monitored every 4 hours using Incucyte® SX5 (Satorius). The Cytotoxicity Index was calculated based on the remaining viable target tumor cells by dividing the total NIR object area of each time point by the value at time 0 for each stimulation.

Cas9 Multi-Guide sgRNA Sequences:

(SEQ ID NO: 93)
AAGACUCUUUAAAGAAGGCA
(SEQ ID NO: 94)
AGUACUCAUUCUCACUGAGU
(SEQ ID NO: 95)
CGUAAAUGCUGAUAUGUAUC
Cas9 Single-guide:
(SEQ ID NO: 98)
TAATCTGGTGGACCTCATGA(AGG)

### Example 13: Roquin-1 KO Enhances the Function of Vδ1 T Cells

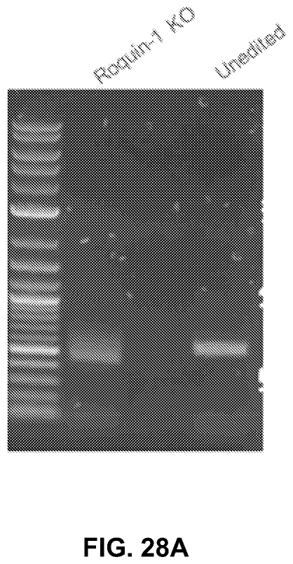

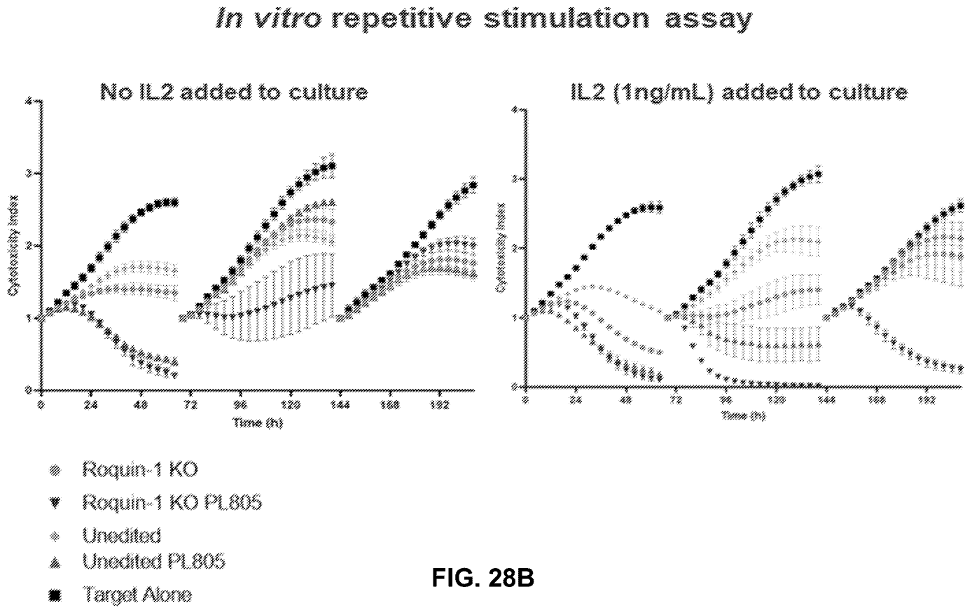

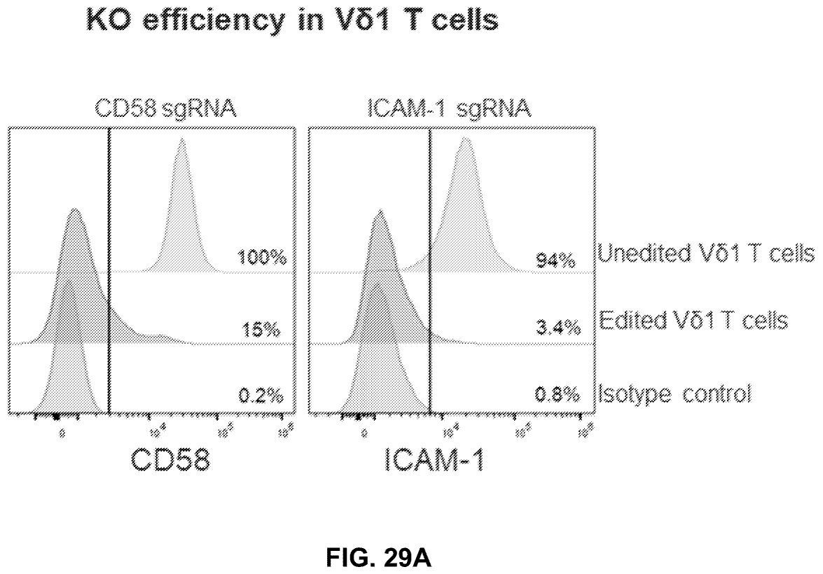

Healthy donor peripheral blood mononuclear cells (PBMCs) were used to activate, expand, CRISPR-gene edit, and engineer Vδ1 γδ T cells to express a CAR. To knockout the Roquin-1 gene in Vδ1 T cells, a ribonucleoprotein (RNP) complex comprising CRISPR-Cas9 (Integrated DNA Technologies) and Roquin-1 sgRNAs were combined with activated Vδ1 T cells from PBMCs (P3 Primary Cell 4D-Nucleofector X Kit L, Lonza). The RNP complex was delivered into Vδ1 T cells by electroporation (Lonza 4D-Nucleofector). Post electroporation, the cells were transferred to T flasks (Corning) containing X-Vivo15 (Lonza) for cell recovery prior to transduction with gammaretroviral vector encoding the CAR and then were subsequently expanded. The results are shown in FIGS. 28A-28B. (FIG. 28A) To determine the Roquin KO efficiency genomic DNA was extracted from cell pellets using the NucleoSpin Tissue kit (MACHARY-NAGEL). The edited region was amplified by PCR using previously in-house optimized primers. The PCR reaction was run on an agarose gel, and the prominent band was excised, and gel extracted using NucleoSpin Gel and PCR Cleanup kit (MACHARY-NAGEL). The extracted DNA was sequenced by Sanger sequencing (Sequetech). % Indels were determined using the ICE Analysis tool (Synthego). Edits were first generated using a 1:1:1 ratio of 3 gRNA sequences to demonstrate the ability to edit the gene in Vd1 T cells, prior to single-guide optimization to identify a gRNA with the highest KO efficiency. (FIG. 28B) To demonstrate enhanced function of Roquin-1 KO CAR-transduced and untransduced Vδ1 T cells, the cytolytic activity against the PSMA+PC3 cell line was evaluated using an in vitro repetitive stimulation assay. PC3-PSMA NIR fluorescently labeled cell line and effector cells were added into a 96-well plate at an E:T ratio of ~3:1 with or without IL-2. Each stimulation was monitored for 3 days after which fresh target cells were plated in another 96-well plate. The effector cells from each stimulation were then transferred into the plate with fresh target cells. The co-cultures were monitored every 4 hours using Incucyte® SX5 (Satorius). The Cytotoxicity Index was calculated based on the remaining viable target tumor cells by dividing the total NIR object area of each time point by the value at time 0 for each stimulation.

Multi-Guide sgRNA Sequences:

(SEQ ID NO: 99)
AAGCCCAUCAGUUUGGGUUG
(SEQ ID NO: 100)
UGUACAAGCUCCACAAUGGA
(SEQ ID NO: 101)
CAAAUGGGCAAGCCUUGCGG
In-house optimized single gRNA sequence:
(SEQ ID NO: 102)
CCTGAATAAACTCCACCGCA(AGG)

### Example 14: ICAM-1 and CD58 KO Enhances the In Vitro Cell Survival of CAR Vδ1 T Cells in the Presence of Allogeneic PBMCs

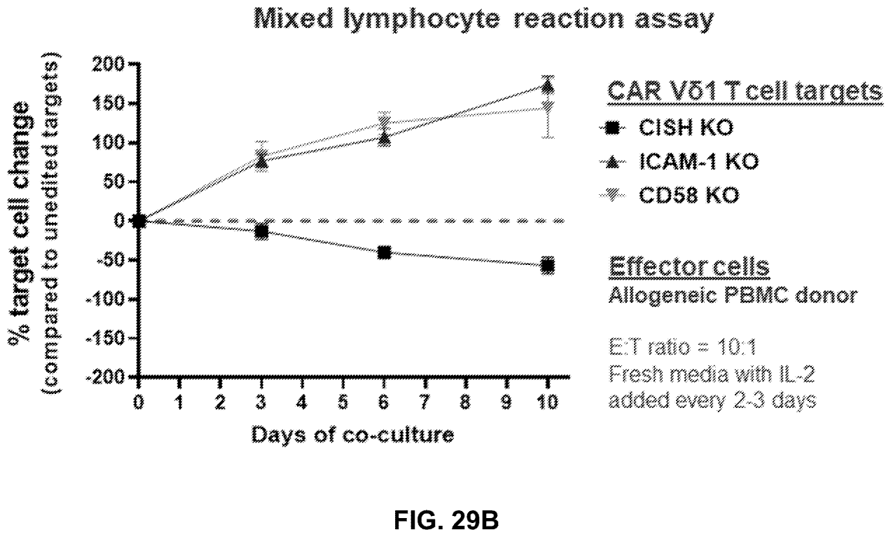

Healthy donor peripheral blood mononuclear cells (PBMCs) were used to activate, expand, CRISPR-gene edit, and engineer Vδ1 γδ T cells to express a CAR. To knockout the CISH gene in Vδ1 T cells, a ribonucleoprotein (RNP) complex comprised of CRISPR-Cas9 (Integrated DNA Technologies) or -MAD7 (Aldevron) and ICAM-1 or CD58 sgRNA were combined with activated Vδ1 T cells from PBMCs (P3 Primary Cell 4D-Nucleofector X Kit L, Lonza). The RNP complex was delivered into Vδ1 T cells by electroporation (Lonza 4D-Nucleofector). Post electroporation, the cells were transferred to T flasks (Corning) containing X-Vivo15 (Lonza) for cell recovery prior to transduction with gammaretroviral vector encoding the CAR and then were subsequently expanded. To determine the KO efficiency of ICAM-1 and CD58, WT (unedited), ICAM-1 KO, and CD58 KO CAR Vδ1 γδ T cells were stained with anti-ICAM-1, anti-CD58, or isotype control antibody conjugated to PE (BioLegend) and acquired using the Novocyte flow cytometer (Agilent Technologies). The results are shown in FIGS. 29A-29B. (FIG. 29A) Greater than 80% KO efficiency was observed for both ICAM-1 and CD58 KO in CAR Vδ1 γδ T cells. (FIG. 29B)

To evaluate if the of ICAM-1 and CD58 can enhance the survival of CAR Vδ1 T cells in the presence of allogeneic PBMCs, a 10-day mixed-lymphocyte reaction assay (MLR) was performed. WT (unedited), CISH KO, ICAM-1 KO, and CD58 KO CAR Vδ1 γδ T cells (target cells) were co-cultured with allogeneic PBMCs (effector cells) at an E:T ratio of 10:1 in the presence of IL-2. During the co-culture, half of the medium was replaced with fresh media containing IL-2 every 2-3 days. At days 3, 6, and 10, cells were harvested from the co-culture and stained for CAR+ and Vδ1+ γδ T cells using B7-H6 conjugated to APC (Adicet Bio generated) for CAR detection and anti-Vδ1 antibody conjugated to PE-CY7 (Adicet Bio generated) for Vδ1 T cell detection. The cells were acquired using the Novocyte flow cytometer to obtain absolute counts of double positive CAR+ Vδ1+ T cells. To determine the % target cell change at each time point, the absolute count of WT CAR+ Vδ1+ γδ T cells was compared to the KO CAR+ Vδ1+ γδ T cell count conditions using the formula: [(WT CAR+ Vδ1+ γδ T cells count−KO CAR+ Vδ1+ γδ T cell count)÷(WT CAR+ Vδ1+ γδ T cells count)]×100. Based on the % target cell change relative to the WT CAR+Vδ1+γδ T cells counts, only the ICAM-1 KO and CD58 KO CAR+ Vδ1+ γδ T cells had an increased % target change, suggesting the disruption of ICAM-1 and CD58 reduced the in vitro killing by allogeneic PBMCs for improved cell survival.

CAS9 ICAM-1 sgRNA:
(SEQ ID NO: 103)
AAAGUCAUCCUGCCCCGGGG
MAD7 ICAM-1 sgRNA:
(SEQ ID NO: 104)
TTTG/AATAGCACATTGGTTGGCTAT
CAS9 CD58 sgRNA:
(SEQ ID NO: 105)
AAAUAUAUGGUGUUGUGUAU
MAD7 CD58 sgRNA:
(SEQ ID NO: 106)
TTTA/GACACTGTGTCAGGTAGCCTC

The embodiments and examples described above are intended to be merely illustrative and non-limiting. Those skilled in the art will recognize or will be able to ascertain using no more than routine experimentation, numerous equivalents of specific compounds, materials and procedures. All such equivalents are considered to be within the scope and are encompassed by the appended claims.

TABLE 2
Sequence information (Amino acid/Nucleotide Sequence)

| SEQ | | |
|---|---|---|
| ID | | |
| No. | Name | Sequence |
| 1 | Full-length | MPEEGSGCSVRRRPYGCVLRAALVPLVAGLVICLVVCIQRF |
| | human CD70 | AQAQQQLPLESLGWDVAELQLNHTGPQQDPRLYWQGGPA |
| | (NCBI | LGRSFLHGPELDKGQLRIHRDGIYMVHIQVTLAICSSTTASR |
| | Accession | HHPTTLAVGICSPASRSISLLRLSFHQGCTIASQRLTPLARGD |
| | NP_001243.1) | TLCTNLTGTLLPSRNTDETFFGVQWVRP |
| | (GI:4507605) | |
| 2 | Signal Peptide | MARPHPWWLCVLGTLVGLS |
| | (amino acid) | |
| 3 | Signal Peptide | ATGGCCAGACCTCATCCTTGGTGGCTGTGTGTGCTGGGC |
| | (nucleotide) | ACACTCGTTGGCCTGTCT |
| 4 | 4-1BB | KRGRKKLLYIFKQPFMRPVQTTQEEDGCSCRFPEEEEGGCE |
| | co-stimulatory | L |
| | domain | |
| | (amino acid) | |
| 5 | 4-1BB | AAACGGGGCAGAAAGAAACTCCTGTATATATTCAAACA |
| | co-stimulatory | ACCATTTATGAGACCAGTACAAACTACTCAAGAGGAAGA |
| | domain | TGGCTGTAGCTGCCGATTTCCAGAAGAAGAAGAAGGAG |
| | (nucleotide) | GATGTGAACTG |
| 6 | Furin P2A | RAKRSGSGATNFSLLKQAGDVEENPGP |
| | (amino acid) | |
| 7 | Furin P2A | CGCGCGAAGCGATCAGGCAGCGGGGCGACAAATTTCAG |
| | (nucleotide) | CCTTCTGAAACAAGCAGGCGACGTGGAAGAAAACCCCG |
| | | GTCCA |
| 8 | P2A cleavage | GSGATNFSLLKQAGDVEENPGP |
| | motif - 1 | |
| | (amino acid) | |
| 9 | P2A cleavage | GGCAGCGGGGCGACAAATTTCAGCCTTCTGAAACAAGCA |
| | motif | GGCGACGTGGAAGAAAACCCCGGTCCA |
| | (nucleotide) | |
| 10 | CD3ζ signaling | RVKFSRSADAPAYQQGQNQLYNELNLGRREEYDVLDKRR |
| | domain-1 | GRDPEMGGKPQRRKNPQEGLYNELQKDKMAEAYSEIGMK |
| | (amino acid) | GERRRGKGHDGLYQGLSTATKDTYDALHMQALPPR |
| 11 | CD3ζ signaling | AGAGTGAAGTTCAGCAGGAGCGCAGACGCCCCCGCGTA |
| | domain -1 | CCAGCAGGGCCAGAACCAGCTCTATAACGAGCTCAATCT |
| | (nucleotide) | AGGACGAAGAGAGGAGTACGATGTTTTGGACAAGAGAC |
| | | GTGGCCGGGACCCTGAGATGGGGGGAAAGCCGCAGAGA |
| | | AGGAAGAACCCTCAGGAAGGCCTGTACAATGAACTGCA |
| | | GAAAGATAAGATGGCGGAGGCCTACAGTGAGATTGGGA |
| | | TGAAAGGCGAGCGCCGGAGGGGCAAGGGGCACGATGGC |
| | | CTTTACCAGGGTCTCAGTACAGCCACCAAGGACACCTAC |
| | | GACGCCCTTCACATGCAGGCCCTGCCCCCTCGC |
| 12 | CD37 signaling | RVKFSRSADAPAYQQGQNQLYNELNLGRREEYDVLDKRR |
| | domain-2 (1XX) | GRDPEMGGKPQRRKNPQEGLFNELQKDKMAEAFSEIGMK |
| | (amino acid) | GERRRGKGHDGLFQGLSTATKDTFDALHMQALPPR |
| 13 | CD33 signaling | RVKFSRSADAPAYQQGQNQLYNELNLGRREEYDVLDKRR |
| | domain-3 | GRDPEMGGKPRRKNPQEGLYNELQKDKMAEAYSEIGMKG |
| | (amino acid) | ERRRGKGHDGLYQGLSTATKDTYDALHMQALPPR |
| 15 | EGFP | MVSKGEELFTGVVPILVELDGDVNGHKFSVSGEGEGDATY |
| | (amino acid) | GKLTLKFICTTGKLPVPWPTLVTTLTYGVQCFSRYPDHMKQ |
| | | HDFFKSAMPEGYVQERTIFFKDDGNYKTRAEVKFEGDTLV |
| | | NRIELKGIDFKEDGNILGHKLEYNYNSHNVYIMADKQKNGI |
| | | KVNFKIRHNIEDGSVQLADHYQQNTPIGDGPVLLPDNHYLS |
| | | TQSALSKDPNEKRDHMVLLEFVTAAGITLGMDELYK |
| 16 | EGFP | ATGGTGAGCAAGGGCGAGGAGCTGTTCACCGGGGTGGT |
| | (nucleotide) | GCCCATCCTGGTCGAGCTGGACGGCGACGTAAACGGCCA |
| | | CAAGTTCAGCGTGTCCGGCGAGGGCGAGGGCGATGCCA |
| | | CCTACGGCAAGCTGACCCTGAAGTTCATCTGCACCACCG |
| | | GCAAGCTGCCCGTGCCCTGGCCCACCCTCGTGACCACCC |
| | | TGACCTACGGCGTGCAGTGCTTCAGCCGCTACCCCGACC |
| | | ACATGAAGCAGCACGACTTCTTCAAGTCCGCCATGCCCG |
| | | AAGGCTACGTCCAGGAGCGCACCATCTTCTTCAAGGACG |
| | | ACGGCAACTACAAGACCCGCGCCGAGGTGAAGTTCGAG |
| | | GGCGACACCCTGGTGAACCGCATCGAGCTGAAGGGCATC |
| | | GACTTCAAGGAGGACGGCAACATCCTGGGGCACAAGCT |
| | | GGAGTACAACTACAACAGCCACAACGTCTATATCATGGC |
| | | CGACAAGCAGAAGAACGGCATCAAGGTGAACTTCAAGA |
| | | TCCGCCACAACATCGAGGACGGCAGCGTGCAGCTCGCCG |
| | | ACCACTACCAGCAGAACACCCCCATCGGCGACGGCCCCG |
| | | TGCTGCTGCCCGACAACCACTACCTGAGCACCCAGTCCG |
| | | CCCTGAGCAAAGACCCCAACGAGAAGCGCGATCACATG |
| | | GTCCTGCTGGAGTTCGTGACCGCCGCCGGGATCACTCTC |
| | | GGCATGGACGAGCTGTACAAGTAA |
| 17 | GMCSFR alpha | MLLLVTSLLLCELPHPAFLLIP |
| | chain signal | |
| | sequence | |
| | (amino acid) | |
| 18 | GMCSFR alpha | atgcttctcctggtgacaagccttctgctctgtgagttaccacacccagcattcctcctgatccca |
| | chain signal | |
| | sequence | |
| | (nucleotide) | |
| 19 | Truncated EGFR | RKVCNGIGIGEFKDSLSINATNIKHFKNCTSISGDLHILPVAF |
| | (EGFRt) | RGDSFTHTPPLDPQELDILKTVKEITGFLLIQAWPENRTDLH |
| | (amino acid) | AFENLEIIRGRTKQHGQFSLAVVSLNITSLGLRSLKEISDGDV |
| | | IISGNKNLCYANTINWKKLFGTSGQKTKIISNRGENSCKATG |
| | | QVCHALCSPEGCWGPEPRDCVSCRNVSRGRECVDKCNLLE |
| | | GEPREFVENSECIQCHPECLPQAMNITCTGRGPDNCIQCAH |
| | | YIDGPHCVKTCPAGVMGENNTLVWKYADAGHVCHLCHPN |
| | | CTYGCTGPGLEGCPTNGPKIPSIATGMVGALLLLLVVALGI |
| | | GLFM |
| 20 | Truncated EGFR | cgcaaagtgtgtaacggaataggtattggtgaatttaaagactcactctccataaatgctacgaa |
| | (EGFRt) | tattaaacacttcaaaaactgcacctccatcagtggcgatctccacatcctgccggtggcattta |
| | (nucleotide) | ggggtgactccttcacacatactcctcctctggatccacaggaactggatattctgaaaaccgta |
| | | aaggaaatcacagggtttttgctgattcaggcttggcctgaaaacaggacggacctccatgcct |
| | | ttgagaacctagaaatcatacgcggcaggaccaagcaacatggtcagttttctcttgcagtcgt |
| | | cagcctgaacataacatccttgggattacgctccctcaaggagataagtgatggagatgtgata |
| | | atttcaggaaacaaaaatttgtgctatgcaaatacaataaactggaaaaaactgtttgggacctc |
| | | cggtcagaaaaccaaaattataagcaacagaggtgaaaacagctgcaaggccacaggccag |
| | | gtctgccatgccttgtgctcccccgagggctgctggggcccggagcccagggactgcgtctc |
| | | ttgccggaatgtcagccgaggcagggaatgcgtggacaagtgcaaccttctggagggtgag |
| | | ccaagggagtttgtggagaactctgagtgcatacagtgccacccagagtgcctgcctcaggc |
| | | catgaacatcacctgcacaggacggggaccagacaactgtatccagtgtgcccactacattga |
| | | cggcccccactgcgtcaagacctgcccggcaggagtcatgggagaaaacaacaccctggtc |
| | | tggaagtacgcagacgccggccatgtgtgccacctgtgccatccaaactgcacctacggatg |
| | | cactgggccaggtcttgaaggctgtccaacgaatgggcctaagatcccgtccatcgccactg |
| | | ggatggtgggggccctcctcttgctgctggtggtggccctggggatcggcctcttcatgTGA |
| | | TAA |
| 21 | dnTGFBR2 | MGRGLLRGLWPLHIVLWTRIAS |
| | signal peptide | |
| | (amino acid) | |
| 22 | dnTGFBR2 | atgggtcgggggctgctcaggggcctgtggccgctgcacatcgtcctgtggacgcgtatcgc |
| | signal peptide | cagc |
| | (nucleotide) | |
| 23 | dnTGFBR2 | TIPPHVQKSVNNDMIVTDNNGAVKFPQLCKFCDVRFSTCD |
| | (amino acid) | NQKSCMSNCSITSICEKPQEVCVAVWRKNDENITLETVCHD |
| | | PKLPYHDFILEDAASPKCIMKEKKKPGETFFMCSCSSDECN |
| | | DNIIFSEEYNTSNPDLLLVIFQVTGISLLPPLGVAISVIIIFYCY |
| | | RVNRQQKLSS |
| 24 | dnTGFBR2 | acgatcccaccgcacgttcagaagtcggttaataacgacatgatagtcactgacaacaacggt |
| | (nucleotide) | gcagtcaagtttccacaactgtgtaaattttgtgatgtgagattttccacctgtgacaaccagaaa |
| | | tcctgcatgagcaactgcagcatcacctccatctgtgagaagccacaggaagtctgtgtggct |
| | | gtatggagaaagaatgacgagaacataacactagagacagtttgccatgaccccaagctccc |
| | | ctaccatgactttattctggaagatgctgcttctccaaagtgcattatgaaggaaaaaaaaaagc |
| | | ctggtgagactttcttcatgtgttcctgtagctctgatgagtgcaatgacaacatcatcttctcaga |
| | | agaatataacaccagcaatcctgacttgttgctagtcatatttcaagtgacaggcatcagcctcct |
| | | gccaccactgggagttgccatatctgtcatcatcatcttctactgctaccgcgttaaccggcagc |
| | | agaagctgagttcatagtaa |
| 25 | LTBR signal | MLLPWATSAPGLAWGPLVLGLFGLLAASQP |
| | peptide | |
| | (amino acid) | |
| 26 | LTBR signal | ATGCTGCTGCCTTGGGCTACATCTGCTCCAGGACTTGCTT |
| | peptide | GGGGACCTCTGGTGCTGGGACTGTTTGGACTGCTGGCTG |
| | (nucleotide) | CCTCTCAGCCT |
| 27 | Full-length | QAVPPYASENQTCRDQEKEYYEPQHRICCSRCPPGTYVSAK |
| | LTBR | CSRIRDTVCATCAENSYNEHWNYLTICQLCRPCDPVMGLEE |
| | (amino acid) | IAPCTSKRKTQCRCQPGMFCAAWALECTHCELLSDCPPGTE |
| | | AELKDEVGKGNNHCVPCKAGHFQNTSSPSARCQPHTRCEN |
| | | QGLVEAAPGTAQSDTTCKNPLEPLPPEMSGTMLMLAVLLP |
| | | LAFFLLLATVFSCIWKSHPSLCRKLGSLLKRRPQGEGPNPV |
| | | AGSWEPPKAHPYFPDLVQPLLPISGDVSPVSTGLPAAPVLE |
| | | AGVPQQQSPLDLTREPQLEPGEQSQVAHGTNGIHVTGGSM |
| | | TITGNIYIYNGPVLGGPPGPGDLPATPEPPYPIPEEGDPGPPG |
| | | LSTPHQEDGKAWHLAETEHCGATPSNRGPRNQFITHD |
| 28 | Full-length | CAAGCCGTTCCTCCATACGCCAGCGAGAACCAGACCTGC |
| | LTBR | CGGGACCAAGAGAAAGAGTACTACGAGCCCCAGCACCG |
| | (nucleotide) | GATCTGCTGTAGCAGATGTCCTCCTGGCACATACGTGTC |
| | | CGCCAAGTGCAGCAGAATCCGGGATACCGTGTGTGCCAC |
| | | CTGTGCCGAGAACAGCTACAACGAGCACTGGAACTACCT |
| | | GACCATCTGCCAGCTGTGCAGGCCCTGCGATCCTGTGAT |
| | | GGGACTCGAGGAAATCGCCCCTTGCACCAGCAAGCGCA |
| | | AGACCCAGTGCAGATGCCAGCCAGGCATGTTCTGTGCCG |
| | | CCTGGGCTCTCGAATGTACCCACTGTGAACTGCTGAGCG |
| | | ACTGCCCTCCTGGAACAGAGGCCGAGCTGAAGGATGAA |
| | | GTCGGCAAGGGCAACAACCACTGCGTGCCATGTAAAGCC |
| | | GGCCACTTCCAGAACACAAGCAGCCCTAGTGCCAGGTGC |
| | | CAGCCTCACACCAGATGCGAGAATCAGGGCCTCGTGGAA |
| | | GCTGCCCCTGGAACTGCTCAGAGCGACACCACATGCAAG |
| | | AACCCTCTGGAACCCCTGCCTCCTGAGATGAGCGGCACA |
| | | ATGCTGATGCTGGCCGTTCTGCTGCCTCTGGCCTTCTTTC |
| | | TGCTGCTGGCCACCGTGTTCAGCTGCATCTGGAAGTCTC |
| | | ACCCCAGCCTGTGCAGAAAGCTGGGCAGCCTGCTTAAGA |
| | | GAAGGCCTCAAGGCGAGGGCCCTAATCCTGTGGCTGGAT |
| | | CTTGGGAGCCTCCTAAGGCTCACCCTTACTTCCCCGATCT |
| | | GGTGCAGCCTCTGCTGCCAATCAGCGGAGATGTGTCCCC |
| | | TGTGTCTACCGGACTTCCTGCCGCTCCTGTTCTTGAAGCT |
| | | GGCGTTCCCCAGCAGCAAAGCCCTCTGGATCTGACCAGA |
| | | GAGCCTCAGCTGGAACCTGGCGAACAGTCTCAAGTGGCC |
| | | CACGGCACAAATGGCATCCATGTGACAGGCGGCAGCAT |
| | | GACCATCACCGGCAACATCTACATCTACAACGGCCCCGT |
| | | GCTCGGCGGACCTCCTGGACCAGGCGATCTTCCTGCTAC |
| | | ACCCGAGCCTCCATATCCTATTCCAGAGGAAGGCGACCC |
| | | TGGACCACCTGGCCTTTCTACACCTCACCAAGAGGATGG |
| | | CAAGGCCTGGCACCTCGCCGAGACAGAACATTGTGGCGC |
| | | TACCCCTAGCAACAGGGGCCCCAGAAATCAGTTCATCAC |
| | | CCACGACTGA |
| 29 | Truncated | MGAGATGRAMDGPRLLLLLLLGVSLGGA |
| | LNGFR signal | |
| | peptide | |
| | (amino acid) | |
| 30 | Truncated | ATGGGAGCTGGTGCTACAGGCAGAGCCATGGATGGACCT |
| | LNGFR signal | AGACTGCTGCTGCTCCTGCTGCTTGGAGTGTCTCTTGGCG |
| | peptide | GAGCC |
| | (nucleotide) | |
| 31 | Truncated | KEACPTGLYTHSGECCKACNLGEGVAQPCGANQTVCEPCL |
| | LNGFR | DSVTFSDVVSATEPCKPCTECVGLQSMSAPCVEADDAVCR |
| | (amino acid) | CAYGYYQDETTGRCEACRVCEAGSGLVFSCQDKQNTVCEE |
| | | CPDGTYSDEANHVDPCLPCTVCEDTERQLRECTRWADAEC |
| | | EEIPGRWITRSTPPEGSDSTAPSTQEPEAPPEQDLIASTVAGV |
| | | VTTVMGSSQPVVTRGTTDNLIPVYCSILAAVVVGLVAYIAF |
| | | KR |
| 32 | Truncated | AAAGAGGCCTGTCCTACCGGCCTGTATACACACTCTGGC |
| | LNGFR | GAGTGCTGCAAGGCCTGCAATCTTGGAGAAGGCGTGGCA |
| | (nucleotide) | CAGCCTTGCGGCGCTAATCAGACAGTGTGCGAGCCTTGC |
| | | CTGGACAGCGTGACCTTTAGCGACGTGGTGTCTGCCACC |
| | | GAGCCATGCAAGCCTTGTACCGAGTGTGTGGGCCTGCAG |
| | | AGCATGTCTGCCCCTTGTGTGGAAGCCGACGATGCCGTG |
| | | TGTAGATGCGCCTACGGCTACTACCAGGACGAGACAACA |
| | | GGCAGATGCGAGGCCTGTAGAGTGTGTGAAGCCGGCTCT |
| | | GGACTGGTGTTCAGCTGCCAAGACAAGCAGAACACCGTG |
| | | TGCGAGGAATGCCCCGATGGCACCTATAGCGACGAGGCC |
| | | AACCATGTGGATCCCTGCCTGCCTTGTACTGTGTGCGAA |
| | | GATACCGAGCGGCAGCTGCGCGAGTGTACAAGATGGGC |
| | | TGATGCCGAGTGCGAAGAGATCCCCGGCAGATGGATCAC |
| | | CAGAAGCACACCTCCAGAGGGCAGCGATAGCACAGCCC |
| | | CTTCTACACAAGAGCCCGAGGCTCCTCCTGAGCAGGATC |
| | | TGATTGCCTCTACAGTGGCCGGCGTGGTCACAACAGTGA |
| | | TGGGATCTTCTCAGCCCGTGGTCACCAGAGGCACCACCG |
| | | ACAATCTGATCCCCGTGTACTGTAGCATCCTGGCCGCCG |
| | | TGGTTGTGGGACTCGTGGCCTATATCGCCTTCAAGAGAT |
| | | GATAA |
| 33 | Full length CD27 | MARPHPWWLCVLGTLVGLSATPAPKSCPERHYWAQGKLC |
| | (amino acid) | CQMCEPGTFLVKDCDQHRKAAQCDPCIPGVSFSPDHHTRP |
| | | HCESCRHCNSGLLVRNCTITANAECACRNGWQCRDKECTE |
| | | CDPLPNPSLTARSSQALSPHPQPTHLPYVSEMLEARTAGHM |
| | | QTLADFRQLPARTLSTHWPPQRSLCSSDFIRILVIFSGMFLVF |
| | | TLAGALFLHQRRKYRSNKGESPVEPAEPCHYSCPREEEGSTI |
| | | PIQEDYRKPEPACSP |
| 34 | Full length CD27 | ATGGCCAGACCTCATCCTTGGTGGCTGTGTGTGCTGGGC |
| | (nucleotide) | ACACTCGTTGGCCTGTCTGCTACACCAGCTCCTAAGAGC |
| | | TGCCCCGAGAGACACTATTGGGCCCAGGGCAAGCTGTGC |
| | | TGCCAGATGTGTGAACCTGGCACCTTCCTGGTCAAGGAC |
| | | TGCGACCAGCACAGAAAGGCCGCTCAGTGCGATCCTTGT |
| | | ATCCCCGGCGTGTCCTTCTCTCCCGACCACCACACAAGA |
| | | CCTCACTGCGAGAGCTGCAGACACTGCAATTCTGGACTG |
| | | CTCGTGCGGAACTGCACCATCACAGCCAATGCCGAGTGC |
| | | GCCTGCAGAAATGGCTGGCAGTGCCGGGACAAAGAATG |
| | | TACCGAGTGCGACCCTCTGCCTAATCCTAGCCTGACCGC |
| | | CAGAAGCAGCCAGGCTTTGTCTCCTCATCCTCAGCCTAC |
| | | ACATCTGCCCTACGTGTCCGAGATGCTGGAAGCCAGAAC |
| | | AGCCGGCCATATGCAGACCCTGGCCGACTTTAGACAGCT |
| | | GCCCGCCAGAACACTGAGCACCCATTGGCCTCCACAGAG |
| | | AAGCCTGTGCAGCAGCGACTTCATCCGGATCCTCGTGAT |
| | | CTTCAGCGGCATGTTCCTGGTGTTCACACTGGCTGGCGCC |
| | | CTGTTTCTGCACCAGCGGAGAAAGTACAGAAGCAACAA |
| | | GGGCGAGAGCCCCGTGGAACCTGCCGAGCCTTGTCACTA |
| | | CAGCTGTCCCAGAGAGGAAGAGGGCAGCACAATCCCCA |
| | | TCCAAGAGGACTACAGAAAGCCCGAGCCTGCCTGCTCTC |
| | | CT |
| 35 | CD27 | ATPAPKSCPERHYWAQGKLCCQMCEPGTFLVKDCDQHRK |
| | extracellular | AAQCDPCIPGVSFSPDHHTRPHCESCRHCNSGLLVRNCTITA |
| | domain | NAECACRNGWQCRDKECTECDPLPNPSLTARSSQALSPH |
| | (amino acid) | PQPTHLPYVSEMLEARTAGHMQTLADFRQLPARTLSTHWP |
| | | PQRSLCSSDFIR |
| 36 | CD27 | GCTACACCAGCTCCTAAGAGCTGCCCCGAGAGACACTAT |
| | extracellular | TGGGCCCAGGGCAAGCTGTGCTGCCAGATGTGTGAACCT |
| | domain | GGCACCTTCCTGGTCAAGGACTGCGACCAGCACAGAAAG |
| | (nucleotide) | GCCGCTCAGTGCGATCCTTGTATCCCCGGCGTGTCCTTCT |
| | | CTCCCGACCACCACACAAGACCTCACTGCGAGAGCTGCA |
| | | GACACTGCAATTCTGGACTGCTCGTGCGGAACTGCACCA |
| | | TCACAGCCAATGCCGAGTGCGCCTGCAGAAATGGCTGGC |
| | | AGTGCCGGGACAAAGAATGTACCGAGTGCGACCCTCTGC |
| | | CTAATCCTAGCCTGACCGCCAGAAGCAGCCAGGCTTTGT |
| | | CTCCTCATCCTCAGCCTACACATCTGCCCTACGTGTCCGA |
| | | GATGCTGGAAGCCAGAACAGCCGGCCATATGCAGACCCT |
| | | GGCCGACTTTAGACAGCTGCCCGCCAGAACACTGAGCAC |
| | | CCATTGGCCTCCACAGAGAAGCCTGTGCAGCAGCGACTT |
| | | CATCCGG |
| 37 | CD27 | ILVIFSGMFLVFTLAGALFLH |
| | transmembrane | |
| | domain | |
| | (amino acid) | |
| 38 | CD27 | ATCCTCGTGATCTTCAGCGGCATGTTCCTGGTGTTCACAC |
| | transmembrane | TGGCTGGCGCCCTGTTTCTGCAC |
| | domain | |
| | (nucleotide) | |
| 39 | CD27 | QRRKYRSNKGESPVEPAEPCHYSCPREEEGSTIPIQEDYRKP |
| | cytoplasmic | EPACSP |
| | domain | |
| | (amino acid) | |
| 40 | CD27 | CAGCGGAGAAAGTACAGAAGCAACAAGGGCGAGAGCCC |
| | cytoplasmic | CGTGGAACCTGCCGAGCCTTGTCACTACAGCTGTCCCAG |
| | domain | AGAGGAAGAGGGCAGCACAATCCCCATCCAAGAGGACT |
| | (nucleotide) | ACAGAAAGCCCGAGCCTGCCTGCTCTCCT |
| 41 | CD27 | ATPAPKSCPERHYWAQGKLCCQMCEPGTFLVKDCDQHRK |
| | extracellular | AAQCDPCIPGVSFSPDHHTWPHCESCRHCNSGLLVRNCTIT |
| | domain (R58W) | ANAECACRNGWQCRDKECTECDPLPNPSLTARSSQALSPH |
| | (amino acid) | PQPTHLPYVSEMLEARTAGHMQTLADFRQLPARTLSTHWP |
| | | PQRSLCSSDFIR |
| 42 | CD27 | GCTACACCAGCTCCTAAGAGCTGCCCCGAGAGACACTAT |
| | extracellular | TGGGCCCAGGGCAAGCTGTGCTGCCAGATGTGTGAACCT |
| | domain (R58W) | GGCACCTTCCTGGTCAAGGACTGCGACCAGCACAGAAAG |
| | (nucleotide) | GCCGCTCAGTGCGATCCTTGTATCCCCGGCGTGTCCTTCT |
| | | CTCCCGACCACCACACATGGCCTCACTGCGAGAGCTGCA |
| | | GACACTGCAATTCTGGACTGCTCGTGCGGAACTGCACCA |
| | | TCACAGCCAATGCCGAGTGCGCCTGCAGAAATGGCTGGC |
| | | AGTGCCGGGACAAAGAATGTACCGAGTGCGACCCTCTGC |
| | | CTAATCCTAGCCTGACCGCCAGAAGCAGCCAGGCTTTGT |
| | | CTCCTCATCCTCAGCCTACACATCTGCCCTACGTGTCCGA |
| | | GATGCTGGAAGCCAGAACAGCCGGCCATATGCAGACCCT |
| | | GGCCGACTTTAGACAGCTGCCCGCCAGAACACTGAGCAC |
| | | CCATTGGCCTCCACAGAGAAGCCTGTGCAGCAGCGACTT |
| | | CATCCGG |
| 43 | Truncated CD27 | MARPHPWWLCVLGTLVGLSATPAPKSCPERHYWAQGKLC |
| | (amino acid) | CQMCEPGTFLVKDCDQHRKAAQCDPCIPGVSFSPDHHTRP |
| | | HCESCRHCNSGLLVRNCTITANAECACRNGWQCRDKECTE |
| | | CDPLPNPSLTARSSQALSPHPQPTHLPYVSEMLEARTAGHM |
| | | QTLADFRQLPARTLSTHWPPQRSLCSSDFIRILVIFSGMFLVF |
| | | TLAGALFLH |
| 44 | Truncated CD27 | ATGGCCAGACCTCATCCTTGGTGGCTGTGTGTGCTGGGC |
| | (nucleotide) | ACACTCGTTGGCCTGTCTGCTACACCAGCTCCTAAGAGC |
| | | TGCCCCGAGAGACACTATTGGGCCCAGGGCAAGCTGTGC |
| | | TGCCAGATGTGTGAACCTGGCACCTTCCTGGTCAAGGAC |
| | | TGCGACCAGCACAGAAAGGCCGCTCAGTGCGATCCTTGT |
| | | ATCCCCGGCGTGTCCTTCTCTCCCGACCACCACACAAGA |
| | | CCTCACTGCGAGAGCTGCAGACACTGCAATTCTGGACTG |
| | | CTCGTGCGGAACTGCACCATCACAGCCAATGCCGAGTGC |
| | | GCCTGCAGAAATGGCTGGCAGTGCCGGGACAAAGAATG |
| | | TACCGAGTGCGACCCTCTGCCTAATCCTAGCCTGACCGC |
| | | CAGAAGCAGCCAGGCTTTGTCTCCTCATCCTCAGCCTAC |
| | | ACATCTGCCCTACGTGTCCGAGATGCTGGAAGCCAGAAC |
| | | AGCCGGCCATATGCAGACCCTGGCCGACTTTAGACAGCT |
| | | GCCCGCCAGAACACTGAGCACCCATTGGCCTCCACAGAG |
| | | AAGCCTGTGCAGCAGCGACTTCATCCGGATCCTCGTGAT |
| | | CTTCAGCGGCATGTTCCTGGTGTTCACACTGGCTGGCGCC |
| | | CTGTTTCTGCAC |
| 45 | PL648 | MARPHPWWLCVLGTLVGLSATPAPKSCPERHYWAQGKLC |
| | (amino acid) | CQMCEPGTFLVKDCDQHRKAAQCDPCIPGVSFSPDHHTRP |
| | | HCESCRHCNSGLLVRNCTITANAECACRNGWQCRDKECTE |
| | | CDPLPNPSLTARSSQALSPHPQPTHLPYVSEMLEARTAGHM |
| | | QTLADFRQLPARTLSTHWPPQRSLCSSDFIRILVIFSGMFLVF |
| | | TLAGALFLHQRRKYRSNKGESPVEPAEPCHYSCPREEEGSTI |
| | | PIQEDYRKPEPACSPKRGRKKLLYIFKQPFMRPVQTTQEED |
| | | GCSCRFPEEEEGGCELRVKFSRSADAPAYQQGQNQLYNEL |
| | | NLGRREEYDVLDKRRGRDPEMGGKPQRRKNPQEGLYNEL |
| | | QKDKMAEAYSEIGMKGERRRGKGHDGLYQGLSTATKDTY |
| | | DALHMQALPPRRAKRSGSGATNFSLLKQAGDVEENPGPM |
| | | VSKGEELFTGVVPILVELDGDVNGHKFSVSGEGEGDATYG |
| | | KLTLKFICTTGKLPVPWPTLVTTLTYGVQCFSRYPDHMKQH |
| | | DFFKSAMPEGYVQERTIFFKDDGNYKTRAEVKFEGDTLVN |
| | | RIELKGIDFKEDGNILGHKLEYNYNSHNVYIMADKQKNGIK |
| | | VNFKIRHNIEDGSVQLADHYQQNTPIGDGPVLLPDNHYLST |
| | | QSALSKDPNEKRDHMVLLEFVTAAGITLGMDELYK |
| 46 | PL648 | ATGGCCAGACCTCATCCTTGGTGGCTGTGTGTGCTGGGC |
| | (nucleotide) | ACACTCGTTGGCCTGTCTGCTACACCAGCTCCTAAGAGC |
| | | TGCCCCGAGAGACACTATTGGGCCCAGGGCAAGCTGTGC |
| | | TGCCAGATGTGTGAACCTGGCACCTTCCTGGTCAAGGAC |
| | | TGCGACCAGCACAGAAAGGCCGCTCAGTGCGATCCTTGT |
| | | ATCCCCGGCGTGTCCTTCTCTCCCGACCACCACACAAGA |
| | | CCTCACTGCGAGAGCTGCAGACACTGCAATTCTGGACTG |
| | | CTCGTGCGGAACTGCACCATCACAGCCAATGCCGAGTGC |
| | | GCCTGCAGAAATGGCTGGCAGTGCCGGGACAAAGAATG |
| | | TACCGAGTGCGACCCTCTGCCTAATCCTAGCCTGACCGC |
| | | CAGAAGCAGCCAGGCTTTGTCTCCTCATCCTCAGCCTAC |
| | | ACATCTGCCCTACGTGTCCGAGATGCTGGAAGCCAGAAC |
| | | AGCCGGCCATATGCAGACCCTGGCCGACTTTAGACAGCT |
| | | GCCCGCCAGAACACTGAGCACCCATTGGCCTCCACAGAG |
| | | AAGCCTGTGCAGCAGCGACTTCATCCGGATCCTCGTGAT |
| | | CTTCAGCGGCATGTTCCTGGTGTTCACACTGGCTGGCGCC |
| | | CTGTTTCTGCACCAGCGGAGAAAGTACAGAAGCAACAA |
| | | GGGCGAGAGCCCCGTGGAACCTGCCGAGCCTTGTCACTA |
| | | CAGCTGTCCCAGAGAGGAAGAGGGCAGCACAATCCCCA |
| | | TCCAAGAGGACTACAGAAAGCCCGAGCCTGCCTGCTCTC |
| | | CTAAACGGGGCAGAAAGAAACTCCTGTATATATTCAAAC |
| | | AACCATTTATGAGACCAGTACAAACTACTCAAGAGGAAG |
| | | ATGGCTGTAGCTGCCGATTTCCAGAAGAAGAAGAAGGA |
| | | GGATGTGAACTGAGAGTGAAGTTCAGCAGGAGCGCAGA |
| | | CGCCCCCGCGTACCAGCAGGGCCAGAACCAGCTCTATAA |
| | | CGAGCTCAATCTAGGACGAAGAGAGGAGTACGATGTTTT |
| | | GGACAAGAGACGTGGCCGGGACCCTGAGATGGGGGGAA |
| | | AGCCGCAGAGAAGGAAGAACCCTCAGGAAGGCCTGTAC |
| | | AATGAACTGCAGAAAGATAAGATGGCGGAGGCCTACAG |
| | | TGAGATTGGGATGAAAGGCGAGCGCCGGAGGGGCAAGG |
| | | GGCACGATGGCCTTTACCAGGGTCTCAGTACAGCCACCA |
| | | AGGACACCTACGACGCCCTTCACATGCAGGCCCTGCCCC |
| | | CTCGCCGCGCGAAGCGATCAGGCAGCGGGGCGACAAAT |
| | | TTCAGCCTTCTGAAACAAGCAGGCGACGTGGAAGAAAA |
| | | CCCCGGTCCAATGGTGAGCAAGGGCGAGGAGCTGTTCAC |
| | | CGGGGTGGTGCCCATCCTGGTCGAGCTGGACGGCGACGT |
| | | AAACGGCCACAAGTTCAGCGTGTCCGGCGAGGGCGAGG |
| | | GCGATGCCACCTACGGCAAGCTGACCCTGAAGTTCATCT |
| | | GCACCACCGGCAAGCTGCCCGTGCCCTGGCCCACCCTCG |
| | | TGACCACCCTGACCTACGGCGTGCAGTGCTTCAGCCGCT |
| | | ACCCCGACCACATGAAGCAGCACGACTTCTTCAAGTCCG |
| | | CCATGCCCGAAGGCTACGTCCAGGAGCGCACCATCTTCT |
| | | TCAAGGACGACGGCAACTACAAGACCCGCGCCGAGGTG |
| | | AAGTTCGAGGGCGACACCCTGGTGAACCGCATCGAGCTG |
| | | AAGGGCATCGACTTCAAGGAGGACGGCAACATCCTGGG |
| | | GCACAAGCTGGAGTACAACTACAACAGCCACAACGTCTA |
| | | TATCATGGCCGACAAGCAGAAGAACGGCATCAAGGTGA |
| | | ACTTCAAGATCCGCCACAACATCGAGGACGGCAGCGTGC |
| | | AGCTCGCCGACCACTACCAGCAGAACACCCCCATCGGCG |
| | | ACGGCCCCGTGCTGCTGCCCGACAACCACTACCTGAGCA |
| | | CCCAGTCCGCCCTGAGCAAAGACCCCAACGAGAAGCGC |
| | | GATCACATGGTCCTGCTGGAGTTCGTGACCGCCGCCGGG |
| | | ATCACTCTCGGCATGGACGAGCTGTACAAGTAA |
| 47 | PL1090 | MARPHPWWLCVLGTLVGLSATPAPKSCPERHYWAQGKLC |
| | (amino acid) | CQMCEPGTFLVKDCDQHRKAAQCDPCIPGVSFSPDHHTRP |
| | | HCESCRHCNSGLLVRNCTITANAECACRNGWQCRDKECTE |
| | | CDPLPNPSLTARSSQALSPHPQPTHLPYVSEMLEARTAGHM |
| | | QTLADFRQLPARTLSTHWPPQRSLCSSDFIRILVIFSGMFLVF |
| | | TLAGALFLHQRRKYRSNKGESPVEPAEPCHYSCPREEEGSTI |
| | | PIQEDYRKPEPACSPKRGRKKLLYIFKQPFMRPVQTTQEED |
| | | GCSCRFPEEEEGGCELRVKFSRSADAPAYQQGQNQLYNEL |
| | | NLGRREEYDVLDKRRGRDPEMGGKPQRRKNPQEGLYNEL |
| | | QKDKMAEAYSEIGMKGERRRGKGHDGLYQGLSTATKDTY |
| | | DALHMQALPPRRAKRSGSGATNFSLLKQAGDVEENPGPML |
| | | LLVTSLLLCELPHPAFLLIPRKVCNGIGIGEFKDSLSINATNIK |
| | | HFKNCTSISGDLHILPVAFRGDSFTHTPPLDPQELDILKTVKE |
| | | ITGFLLIQAWPENRTDLHAFENLEIIRGRTKQHGQFSLAVVS |
| | | LNITSLGLRSLKEISDGDVIISGNKNLCYANTINWKKLFGTS |
| | | GQKTKIISNRGENSCKATGQVCHALCSPEGCWGPEPRDCVS |
| | | CRNVSRGRECVDKCNLLEGEPREFVENSECIQCHPECLPQA |
| | | MNITCTGRGPDNCIQCAHYIDGPHCVKTCPAGVMGENNTL |
| | | VWKYADAGHVCHLCHPNCTYGCTGPGLEGCPTNGPKIPSI |
| | | ATGMVGALLLLLVVALGIGLFM |
| 48 | PL1090 | ATGGCCAGACCTCATCCTTGGTGGCTGTGTGTGCTGGGC |
| | (nucleotide) | ACACTCGTTGGCCTGTCTGCTACACCAGCTCCTAAGAGC |
| | | TGCCCCGAGAGACACTATTGGGCCCAGGGCAAGCTGTGC |
| | | TGCCAGATGTGTGAACCTGGCACCTTCCTGGTCAAGGAC |
| | | TGCGACCAGCACAGAAAGGCCGCTCAGTGCGATCCTTGT |
| | | ATCCCCGGCGTGTCCTTCTCTCCCGACCACCACACAAGA |
| | | CCTCACTGCGAGAGCTGCAGACACTGCAATTCTGGACTG |
| | | CTCGTGCGGAACTGCACCATCACAGCCAATGCCGAGTGC |
| | | GCCTGCAGAAATGGCTGGCAGTGCCGGGACAAAGAATG |
| | | TACCGAGTGCGACCCTCTGCCTAATCCTAGCCTGACCGC |
| | | CAGAAGCAGCCAGGCTTTGTCTCCTCATCCTCAGCCTAC |
| | | ACATCTGCCCTACGTGTCCGAGATGCTGGAAGCCAGAAC |
| | | AGCCGGCCATATGCAGACCCTGGCCGACTTTAGACAGCT |
| | | GCCCGCCAGAACACTGAGCACCCATTGGCCTCCACAGAG |
| | | AAGCCTGTGCAGCAGCGACTTCATCCGGATCCTCGTGAT |
| | | CTTCAGCGGCATGTTCCTGGTGTTCACACTGGCTGGCGCC |
| | | CTGTTTCTGCACCAGCGGAGAAAGTACAGAAGCAACAA |
| | | GGGCGAGAGCCCCGTGGAACCTGCCGAGCCTTGTCACTA |
| | | CAGCTGTCCCAGAGAGGAAGAGGGCAGCACAATCCCCA |
| | | TCCAAGAGGACTACAGAAAGCCCGAGCCTGCCTGCTCTC |
| | | CTAAACGGGGCAGAAAGAAACTCCTGTATATATTCAAAC |
| | | AACCATTTATGAGACCAGTACAAACTACTCAAGAGGAAG |
| | | ATGGCTGTAGCTGCCGATTTCCAGAAGAAGAAGAAGGA |
| | | GGATGTGAACTGAGAGTGAAGTTCAGCAGGAGCGCAGA |
| | | CGCCCCCGCGTACCAGCAGGGCCAGAACCAGCTCTATAA |
| | | CGAGCTCAATCTAGGACGAAGAGAGGAGTACGATGTTTT |
| | | GGACAAGAGACGTGGCCGGGACCCTGAGATGGGGGGAA |
| | | AGCCGCAGAGAAGGAAGAACCCTCAGGAAGGCCTGTAC |
| | | AATGAACTGCAGAAAGATAAGATGGCGGAGGCCTACAG |
| | | TGAGATTGGGATGAAAGGCGAGCGCCGGAGGGGCAAGG |
| | | GGCACGATGGCCTTTACCAGGGTCTCAGTACAGCCACCA |
| | | AGGACACCTACGACGCCCTTCACATGCAGGCCCTGCCCC |
| | | CTCGCCGCGCGAAGCGATCAGGCAGCGGGGCGACAAAT |
| | | TTCAGCCTTCTGAAACAAGCAGGCGACGTGGAAGAAAA |
| | | CCCCGGTCCAatgcttctcctggtgacaagccttctgctctgtgagttaccacacccag |
| | | cattcctcctgatcccacgcaaagtgtgtaacggaataggtattggtgaatttaaagactcactct |
| | | ccataaatgctacgaatattaaacacttcaaaaactgcacctccatcagtggcgatctccacatc |
| | | ctgccggtggcatttaggggtgactccttcacacatactcctcctctggatccacaggaactgg |
| | | atattctgaaaaccgtaaaggaaatcacagggtttttgctgattcaggcttggcctgaaaacagg |
| | | acggacctccatgcctttgagaacctagaaatcatacgcggcaggaccaagcaacatggtca |
| | | gttttctcttgcagtcgtcagcctgaacataacatccttgggattacgctccctcaaggagataag |
| | | tgatggagatgtgataatttcaggaaacaaaaatttgtgctatgcaaatacaataaactggaaaa |
| | | aactgtttgggacctccggtcagaaaaccaaaattataagcaacagaggtgaaaacagctgca |
| | | aggccacaggccaggtctgccatgccttgtgctcccccgagggctgctggggcccggagcc |
| | | cagggactgcgtctcttgccggaatgtcagccgaggcagggaatgcgtggacaagtgcaac |
| | | cttctggagggtgagccaagggagtttgtggagaactctgagtgcatacagtgccacccaga |
| | | gtgcctgcctcaggccatgaacatcacctgcacaggacggggaccagacaactgtatccagt |
| | | gtgcccactacattgacggcccccactgcgtcaagacctgcccggcaggagtcatgggaga |
| | | aaacaacaccctggtctggaagtacgcagacgccggccatgtgtgccacctgtgccatccaa |
| | | actgcacctacggatgcactgggccaggtcttgaaggctgtccaacgaatgggcctaagatc |
| | | ccgtccatcgccactgggatggtgggggccctcctcttgctgctggtggtggccctggggatc |
| | | ggcctcttcatgTGATAA |
| 49 | PL1093 | MARPHPWWLCVLGTLVGLSATPAPKSCPERHYWAQGKLC |
| | (amino acid) | CQMCEPGTFLVKDCDQHRKAAQCDPCIPGVSFSPDHHTRP |
| | | HCESCRHCNSGLLVRNCTITANAECACRNGWQCRDKECTE |
| | | CDPLPNPSLTARSSQALSPHPQPTHLPYVSEMLEARTAGHM |
| | | QTLADFRQLPARTLSTHWPPQRSLCSSDFIRILVIFSGMFLVF |
| | | TLAGALFLHQRRKYRSNKGESPVEPAEPCHYSCPREEEGSTI |
| | | PIQEDYRKPEPACSPKRGRKKLLYIFKQPFMRPVQTTQEED |
| | | GCSCRFPEEEEGGCELRVKFSRSADAPAYQQGQNQLYNEL |
| | | NLGRREEYDVLDKRRGRDPEMGGKPQRRKNPQEGLYNEL |
| | | QKDKMAEAYSEIGMKGERRRGKGHDGLYQGLSTATKDTY |
| | | DALHMQALPPRRAKRSGSGATNFSLLKQAGDVEENPGPM |
| | | GRGLLRGLWPLHIVLWTRIASTIPPHVQKSVNNDMIVTDNN |
| | | GAVKFPQLCKFCDVRFSTCDNQKSCMSNCSITSICEKPQEV |
| | | CVAVWRKNDENITLETVCHDPKLPYHDFILEDAASPKCIMK |
| | | EKKKPGETFFMCSCSSDECNDNIIFSEEYNTSNPDLLLVIFQ |
| | | VTGISLLPPLGVAISVIIIFYCYRVNRQQKLSS |
| 50 | PL1093 | ATGGCCAGACCTCATCCTTGGTGGCTGTGTGTGCTGGGC |
| | (nucleotide) | ACACTCGTTGGCCTGTCTGCTACACCAGCTCCTAAGAGC |
| | | TGCCCCGAGAGACACTATTGGGCCCAGGGCAAGCTGTGC |
| | | TGCCAGATGTGTGAACCTGGCACCTTCCTGGTCAAGGAC |
| | | TGCGACCAGCACAGAAAGGCCGCTCAGTGCGATCCTTGT |
| | | ATCCCCGGCGTGTCCTTCTCTCCCGACCACCACACAAGA |
| | | CCTCACTGCGAGAGCTGCAGACACTGCAATTCTGGACTG |
| | | CTCGTGCGGAACTGCACCATCACAGCCAATGCCGAGTGC |
| | | GCCTGCAGAAATGGCTGGCAGTGCCGGGACAAAGAATG |
| | | TACCGAGTGCGACCCTCTGCCTAATCCTAGCCTGACCGC |
| | | CAGAAGCAGCCAGGCTTTGTCTCCTCATCCTCAGCCTAC |
| | | ACATCTGCCCTACGTGTCCGAGATGCTGGAAGCCAGAAC |
| | | AGCCGGCCATATGCAGACCCTGGCCGACTTTAGACAGCT |
| | | GCCCGCCAGAACACTGAGCACCCATTGGCCTCCACAGAG |
| | | AAGCCTGTGCAGCAGCGACTTCATCCGGATCCTCGTGAT |
| | | CTTCAGCGGCATGTTCCTGGTGTTCACACTGGCTGGCGCC |
| | | CTGTTTCTGCACCAGCGGAGAAAGTACAGAAGCAACAA |
| | | GGGCGAGAGCCCCGTGGAACCTGCCGAGCCTTGTCACTA |
| | | CAGCTGTCCCAGAGAGGAAGAGGGCAGCACAATCCCCA |
| | | TCCAAGAGGACTACAGAAAGCCCGAGCCTGCCTGCTCTC |
| | | CTAAACGGGGCAGAAAGAAACTCCTGTATATATTCAAAC |
| | | AACCATTTATGAGACCAGTACAAACTACTCAAGAGGAAG |
| | | ATGGCTGTAGCTGCCGATTTCCAGAAGAAGAAGAAGGA |
| | | GGATGTGAACTGAGAGTGAAGTTCAGCAGGAGCGCAGA |
| | | CGCCCCCGCGTACCAGCAGGGCCAGAACCAGCTCTATAA |
| | | CGAGCTCAATCTAGGACGAAGAGAGGAGTACGATGTTTT |
| | | GGACAAGAGACGTGGCCGGGACCCTGAGATGGGGGGAA |
| | | AGCCGCAGAGAAGGAAGAACCCTCAGGAAGGCCTGTAC |
| | | AATGAACTGCAGAAAGATAAGATGGCGGAGGCCTACAG |
| | | TGAGATTGGGATGAAAGGCGAGCGCCGGAGGGGCAAGG |
| | | GGCACGATGGCCTTTACCAGGGTCTCAGTACAGCCACCA |
| | | AGGACACCTACGACGCCCTTCACATGCAGGCCCTGCCCC |
| | | CTCGCCGCGCGAAGCGATCAGGCAGCGGGGCGACAAAT |
| | | TTCAGCCTTCTGAAACAAGCAGGCGACGTGGAAGAAAA |
| | | CCCCGGTCCAatgggtcgggggctgctcaggggcctgtggccgctgcacatcgtcc |
| | | tgtggacgcgtatcgccagcacgatcccaccgcacgttcagaagtcggttaataacgacatga |
| | | tagtcactgacaacaacggtgcagtcaagtttccacaactgtgtaaattttgtgatgtgagattttc |
| | | cacctgtgacaaccagaaatcctgcatgagcaactgcagcatcacctccatctgtgagaagcc |
| | | acaggaagtctgtgtggctgtatggagaaagaatgacgagaacataacactagagacagtttg |
| | | ccatgaccccaagctcccctaccatgactttattctggaagatgctgcttctccaaagtgcattat |
| | | gaaggaaaaaaaaaagcctggtgagactttcttcatgtgttcctgtagctctgatgagtgcaatg |
| | | acaacatcatcttctcagaagaatataacaccagcaatcctgacttgttgctagtcatatttcaagt |
| | | gacaggcatcagcctcctgccaccactgggagttgccatatctgtcatcatcatcttctactgct |
| | | accgcgttaaccggcagcagaagctgagttcatagtaa |
| 51 | PL1106 | MARPHPWWLCVLGTLVGLSATPAPKSCPERHYWAQGKLC |
| | (amino acid) | CQMCEPGTFLVKDCDQHRKAAQCDPCIPGVSFSPDHHTRP |
| | | HCESCRHCNSGLLVRNCTITANAECACRNGWQCRDKECTE |
| | | CDPLPNPSLTARSSQALSPHPQPTHLPYVSEMLEARTAGHM |
| | | QTLADFRQLPARTLSTHWPPQRSLCSSDFIRILVIFSGMFLVF |
| | | TLAGALFLHQRRKYRSNKGESPVEPAEPCHYSCPREEEGSTI |
| | | PIQEDYRKPEPACSPKRGRKKLLYIFKQPFMRPVQTTQEED |
| | | GCSCRFPEEEEGGCELRVKFSRSADAPAYQQGQNQLYNEL |
| | | NLGRREEYDVLDKRRGRDPEMGGKPQRRKNPQEGLYNEL |
| | | QKDKMAEAYSEIGMKGERRRGKGHDGLYQGLSTATKDTY |
| | | DALHMQALPPRRAKRSGSGATNFSLLKQAGDVEENPGPML |
| | | LPWATSAPGLAWGPLVLGLFGLLAASQPQAVPPYASENQT |
| | | CRDQEKEYYEPQHRICCSRCPPGTYVSAKCSRIRDTVCATC |
| | | AENSYNEHWNYLTICQLCRPCDPVMGLEEIAPCTSKRKTQC |
| | | RCQPGMFCAAWALECTHCELLSDCPPGTEAELKDEVGKGN |
| | | NHCVPCKAGHFQNTSSPSARCQPHTRCENQGLVEAAPGTA |
| | | QSDTTCKNPLEPLPPEMSGTMLMLAVLLPLAFFLLLATVFS |
| | | CIWKSHPSLCRKLGSLLKRRPQGEGPNPVAGSWEPPKAHPY |
| | | FPDLVQPLLPISGDVSPVSTGLPAAPVLEAGVPQQQSPLDLT |
| | | REPQLEPGEQSQVAHGTNGIHVTGGSMTITGNIYIYNGPVL |
| | | GGPPGPGDLPATPEPPYPIPEEGDPGPPGLSTPHQEDGKAW |
| | | HLAETEHCGATPSNRGPRNQFITHD |
| 52 | PL1106 | ATGGCCAGACCTCATCCTTGGTGGCTGTGTGTGCTGGGC |
| | (nucleotide) | ACACTCGTTGGCCTGTCTGCTACACCAGCTCCTAAGAGC |
| | | TGCCCCGAGAGACACTATTGGGCCCAGGGCAAGCTGTGC |
| | | TGCCAGATGTGTGAACCTGGCACCTTCCTGGTCAAGGAC |
| | | TGCGACCAGCACAGAAAGGCCGCTCAGTGCGATCCTTGT |
| | | ATCCCCGGCGTGTCCTTCTCTCCCGACCACCACACAAGA |
| | | CCTCACTGCGAGAGCTGCAGACACTGCAATTCTGGACTG |
| | | CTCGTGCGGAACTGCACCATCACAGCCAATGCCGAGTGC |
| | | GCCTGCAGAAATGGCTGGCAGTGCCGGGACAAAGAATG |
| | | TACCGAGTGCGACCCTCTGCCTAATCCTAGCCTGACCGC |
| | | CAGAAGCAGCCAGGCTTTGTCTCCTCATCCTCAGCCTAC |
| | | ACATCTGCCCTACGTGTCCGAGATGCTGGAAGCCAGAAC |
| | | AGCCGGCCATATGCAGACCCTGGCCGACTTTAGACAGCT |
| | | GCCCGCCAGAACACTGAGCACCCATTGGCCTCCACAGAG |
| | | AAGCCTGTGCAGCAGCGACTTCATCCGGATCCTCGTGAT |
| | | CTTCAGCGGCATGTTCCTGGTGTTCACACTGGCTGGCGCC |
| | | CTGTTTCTGCACCAGCGGAGAAAGTACAGAAGCAACAA |
| | | GGGCGAGAGCCCCGTGGAACCTGCCGAGCCTTGTCACTA |
| | | CAGCTGTCCCAGAGAGGAAGAGGGCAGCACAATCCCCA |
| | | TCCAAGAGGACTACAGAAAGCCCGAGCCTGCCTGCTCTC |
| | | CTAAACGGGGCAGAAAGAAACTCCTGTATATATTCAAAC |
| | | AACCATTTATGAGACCAGTACAAACTACTCAAGAGGAAG |
| | | ATGGCTGTAGCTGCCGATTTCCAGAAGAAGAAGAAGGA |
| | | GGATGTGAACTGAGAGTGAAGTTCAGCAGGAGCGCAGA |
| | | CGCCCCCGCGTACCAGCAGGGCCAGAACCAGCTCTATAA |
| | | CGAGCTCAATCTAGGACGAAGAGAGGAGTACGATGTTTT |
| | | GGACAAGAGACGTGGCCGGGACCCTGAGATGGGGGGAA |
| | | AGCCGCAGAGAAGGAAGAACCCTCAGGAAGGCCTGTAC |
| | | AATGAACTGCAGAAAGATAAGATGGCGGAGGCCTACAG |
| | | TGAGATTGGGATGAAAGGCGAGCGCCGGAGGGGCAAGG |
| | | GGCACGATGGCCTTTACCAGGGTCTCAGTACAGCCACCA |
| | | AGGACACCTACGACGCCCTTCACATGCAGGCCCTGCCCC |
| | | CTCGCCGCGCGAAGCGATCAGGCAGCGGGGCGACAAAT |
| | | TTCAGCCTTCTGAAACAAGCAGGCGACGTGGAAGAAAA |
| | | CCCCGGTCCAATGCTGCTGCCTTGGGCTACATCTGCTCCA |
| | | GGACTTGCTTGGGGACCTCTGGTGCTGGGACTGTTTGGA |
| | | CTGCTGGCTGCCTCTCAGCCTCAAGCCGTTCCTCCATACG |
| | | CCAGCGAGAACCAGACCTGCCGGGACCAAGAGAAAGAG |
| | | TACTACGAGCCCCAGCACCGGATCTGCTGTAGCAGATGT |
| | | CCTCCTGGCACATACGTGTCCGCCAAGTGCAGCAGAATC |
| | | CGGGATACCGTGTGTGCCACCTGTGCCGAGAACAGCTAC |
| | | AACGAGCACTGGAACTACCTGACCATCTGCCAGCTGTGC |
| | | AGGCCCTGCGATCCTGTGATGGGACTCGAGGAAATCGCC |
| | | CCTTGCACCAGCAAGCGCAAGACCCAGTGCAGATGCCA |
| | | GCCAGGCATGTTCTGTGCCGCCTGGGCTCTCGAATGTAC |
| | | CCACTGTGAACTGCTGAGCGACTGCCCTCCTGGAACAGA |
| | | GGCCGAGCTGAAGGATGAAGTCGGCAAGGGCAACAACC |
| | | ACTGCGTGCCATGTAAAGCCGGCCACTTCCAGAACACAA |
| | | GCAGCCCTAGTGCCAGGTGCCAGCCTCACACCAGATGCG |
| | | AGAATCAGGGCCTCGTGGAAGCTGCCCCTGGAACTGCTC |
| | | AGAGCGACACCACATGCAAGAACCCTCTGGAACCCCTGC |
| | | CTCCTGAGATGAGCGGCACAATGCTGATGCTGGCCGTTC |
| | | TGCTGCCTCTGGCCTTCTTTCTGCTGCTGGCCACCGTGTT |
| | | CAGCTGCATCTGGAAGTCTCACCCCAGCCTGTGCAGAAA |
| | | GCTGGGCAGCCTGCTTAAGAGAAGGCCTCAAGGCGAGG |
| | | GCCCTAATCCTGTGGCTGGATCTTGGGAGCCTCCTAAGG |
| | | CTCACCCTTACTTCCCCGATCTGGTGCAGCCTCTGCTGCC |
| | | AATCAGCGGAGATGTGTCCCCTGTGTCTACCGGACTTCC |
| | | TGCCGCTCCTGTTCTTGAAGCTGGCGTTCCCCAGCAGCA |
| | | AAGCCCTCTGGATCTGACCAGAGAGCCTCAGCTGGAACC |
| | | TGGCGAACAGTCTCAAGTGGCCCACGGCACAAATGGCAT |
| | | CCATGTGACAGGCGGCAGCATGACCATCACCGGCAACAT |
| | | CTACATCTACAACGGCCCCGTGCTCGGCGGACCTCCTGG |
| | | ACCAGGCGATCTTCCTGCTACACCCGAGCCTCCATATCC |
| | | TATTCCAGAGGAAGGCGACCCTGGACCACCTGGCCTTTC |
| | | TACACCTCACCAAGAGGATGGCAAGGCCTGGCACCTCGC |
| | | CGAGACAGAACATTGTGGCGCTACCCCTAGCAACAGGG |
| | | GCCCCAGAAATCAGTTCATCACCCACGACTGA |
| 53 | PL1107 | MARPHPWWLCVLGTLVGLSATPAPKSCPERHYWAQGKLC |
| | (amino acid) | CQMCEPGTFLVKDCDQHRKAAQCDPCIPGVSFSPDHHTRP |
| | | HCESCRHCNSGLLVRNCTITANAECACRNGWQCRDKECTE |
| | | CDPLPNPSLTARSSQALSPHPQPTHLPYVSEMLEARTAGHM |
| | | QTLADFRQLPARTLSTHWPPQRSLCSSDFIRILVIFSGMFLVF |
| | | TLAGALFLHQRRKYRSNKGESPVEPAEPCHYSCPREEEGSTI |
| | | PIQEDYRKPEPACSPKRGRKKLLYIFKQPFMRPVQTTQEED |
| | | GCSCRFPEEEEGGCELRVKFSRSADAPAYQQGQNQLYNEL |
| | | NLGRREEYDVLDKRRGRDPEMGGKPQRRKNPQEGLYNEL |
| | | QKDKMAEAYSEIGMKGERRRGKGHDGLYQGLSTATKDTY |
| | | DALHMQALPPR |
| 54 | PL1107 | ATGGCCAGACCTCATCCTTGGTGGCTGTGTGTGCTGGGC |
| | (nucleotide) | ACACTCGTTGGCCTGTCTGCTACACCAGCTCCTAAGAGC |
| | | TGCCCCGAGAGACACTATTGGGCCCAGGGCAAGCTGTGC |
| | | TGCCAGATGTGTGAACCTGGCACCTTCCTGGTCAAGGAC |
| | | TGCGACCAGCACAGAAAGGCCGCTCAGTGCGATCCTTGT |
| | | ATCCCCGGCGTGTCCTTCTCTCCCGACCACCACACAAGA |
| | | CCTCACTGCGAGAGCTGCAGACACTGCAATTCTGGACTG |
| | | CTCGTGCGGAACTGCACCATCACAGCCAATGCCGAGTGC |
| | | GCCTGCAGAAATGGCTGGCAGTGCCGGGACAAAGAATG |
| | | TACCGAGTGCGACCCTCTGCCTAATCCTAGCCTGACCGC |
| | | CAGAAGCAGCCAGGCTTTGTCTCCTCATCCTCAGCCTAC |
| | | ACATCTGCCCTACGTGTCCGAGATGCTGGAAGCCAGAAC |
| | | AGCCGGCCATATGCAGACCCTGGCCGACTTTAGACAGCT |
| | | GCCCGCCAGAACACTGAGCACCCATTGGCCTCCACAGAG |
| | | AAGCCTGTGCAGCAGCGACTTCATCCGGATCCTCGTGAT |
| | | CTTCAGCGGCATGTTCCTGGTGTTCACACTGGCTGGCGCC |
| | | CTGTTTCTGCACCAGCGGAGAAAGTACAGAAGCAACAA |
| | | GGGCGAGAGCCCCGTGGAACCTGCCGAGCCTTGTCACTA |
| | | CAGCTGTCCCAGAGAGGAAGAGGGCAGCACAATCCCCA |
| | | TCCAAGAGGACTACAGAAAGCCCGAGCCTGCCTGCTCTC |
| | | CTAAACGGGGCAGAAAGAAACTCCTGTATATATTCAAAC |
| | | AACCATTTATGAGACCAGTACAAACTACTCAAGAGGAAG |
| | | ATGGCTGTAGCTGCCGATTTCCAGAAGAAGAAGAAGGA |
| | | GGATGTGAACTGAGAGTGAAGTTCAGCAGGAGCGCAGA |
| | | CGCCCCCGCGTACCAGCAGGGCCAGAACCAGCTCTATAA |
| | | CGAGCTCAATCTAGGACGAAGAGAGGAGTACGATGTTTT |
| | | GGACAAGAGACGTGGCCGGGACCCTGAGATGGGGGGAA |
| | | AGCCGCAGAGAAGGAAGAACCCTCAGGAAGGCCTGTAC |
| | | AATGAACTGCAGAAAGATAAGATGGCGGAGGCCTACAG |
| | | TGAGATTGGGATGAAAGGCGAGCGCCGGAGGGGCAAGG |
| | | GGCACGATGGCCTTTACCAGGGTCTCAGTACAGCCACCA |
| | | AGGACACCTACGACGCCCTTCACATGCAGGCCCTGCCCC |
| | | CTCGCTAA |
| 55 | PL1110 | MARPHPWWLCVLGTLVGLSATPAPKSCPERHYWAQGKLC |
| | (amino acid) | CQMCEPGTFLVKDCDQHRKAAQCDPCIPGVSFSPDHHTRP |
| | | HCESCRHCNSGLLVRNCTITANAECACRNGWQCRDKECTE |
| | | CDPLPNPSLTARSSQALSPHPQPTHLPYVSEMLEARTAGHM |
| | | QTLADFRQLPARTLSTHWPPQRSLCSSDFIRILVIFSGMFLVF |
| | | TLAGALFLHQRRKYRSNKGESPVEPAEPCHYSCPREEEGSTI |
| | | PIQEDYRKPEPACSPKRGRKKLLYIFKQPFMRPVQTTQEED |
| | | GCSCRFPEEEEGGCELRVKFSRSADAPAYQQGQNQLYNEL |
| | | NLGRREEYDVLDKRRGRDPEMGGKPQRRKNPQEGLYNEL |
| | | QKDKMAEAYSEIGMKGERRRGKGHDGLYQGLSTATKDTY |
| | | DALHMQALPPRRAKRSGSGATNFSLLKQAGDVEENPGPM |
| | | GAGATGRAMDGPRLLLLLLLGVSLGGAKEACPTGLYTHSG |
| | | ECCKACNLGEGVAQPCGANQTVCEPCLDSVTFSDVVSATE |
| | | PCKPCTECVGLQSMSAPCVEADDAVCRCAYGYYQDETTG |
| | | RCEACRVCEAGSGLVFSCQDKQNTVCEECPDGTYSDEANH |
| | | VDPCLPCTVCEDTERQLRECTRWADAECEEIPGRWITRSTP |
| | | PEGSDSTAPSTQEPEAPPEQDLIASTVAGVVTTVMGSSQPV |
| | | VTRGTTDNLIPVYCSILAAVVVGLVAYIAFKR |
| 56 | PL1110 | ATGGCCAGACCTCATCCTTGGTGGCTGTGTGTGCTGGGC |
| | (nucleotide) | ACACTCGTTGGCCTGTCTGCTACACCAGCTCCTAAGAGC |
| | | TGCCCCGAGAGACACTATTGGGCCCAGGGCAAGCTGTGC |
| | | TGCCAGATGTGTGAACCTGGCACCTTCCTGGTCAAGGAC |
| | | TGCGACCAGCACAGAAAGGCCGCTCAGTGCGATCCTTGT |
| | | ATCCCCGGCGTGTCCTTCTCTCCCGACCACCACACAAGA |
| | | CCTCACTGCGAGAGCTGCAGACACTGCAATTCTGGACTG |
| | | CTCGTGCGGAACTGCACCATCACAGCCAATGCCGAGTGC |
| | | GCCTGCAGAAATGGCTGGCAGTGCCGGGACAAAGAATG |
| | | TACCGAGTGCGACCCTCTGCCTAATCCTAGCCTGACCGC |
| | | CAGAAGCAGCCAGGCTTTGTCTCCTCATCCTCAGCCTAC |
| | | ACATCTGCCCTACGTGTCCGAGATGCTGGAAGCCAGAAC |
| | | AGCCGGCCATATGCAGACCCTGGCCGACTTTAGACAGCT |
| | | GCCCGCCAGAACACTGAGCACCCATTGGCCTCCACAGAG |
| | | AAGCCTGTGCAGCAGCGACTTCATCCGGATCCTCGTGAT |
| | | CTTCAGCGGCATGTTCCTGGTGTTCACACTGGCTGGCGCC |
| | | CTGTTTCTGCACCAGCGGAGAAAGTACAGAAGCAACAA |
| | | GGGCGAGAGCCCCGTGGAACCTGCCGAGCCTTGTCACTA |
| | | CAGCTGTCCCAGAGAGGAAGAGGGCAGCACAATCCCCA |
| | | TCCAAGAGGACTACAGAAAGCCCGAGCCTGCCTGCTCTC |
| | | CTAAACGGGGCAGAAAGAAACTCCTGTATATATTCAAAC |
| | | AACCATTTATGAGACCAGTACAAACTACTCAAGAGGAAG |
| | | ATGGCTGTAGCTGCCGATTTCCAGAAGAAGAAGAAGGA |
| | | GGATGTGAACTGAGAGTGAAGTTCAGCAGGAGCGCAGA |
| | | CGCCCCCGCGTACCAGCAGGGCCAGAACCAGCTCTATAA |
| | | CGAGCTCAATCTAGGACGAAGAGAGGAGTACGATGTTTT |
| | | GGACAAGAGACGTGGCCGGGACCCTGAGATGGGGGGAA |
| | | AGCCGCAGAGAAGGAAGAACCCTCAGGAAGGCCTGTAC |
| | | AATGAACTGCAGAAAGATAAGATGGCGGAGGCCTACAG |
| | | TGAGATTGGGATGAAAGGCGAGCGCCGGAGGGGCAAGG |
| | | GGCACGATGGCCTTTACCAGGGTCTCAGTACAGCCACCA |
| | | AGGACACCTACGACGCCCTTCACATGCAGGCCCTGCCCC |
| | | CTCGCCGCGCGAAGCGATCAGGCAGCGGGGCGACAAAT |
| | | TTCAGCCTTCTGAAACAAGCAGGCGACGTGGAAGAAAA |
| | | CCCCGGTCCAATGGGAGCTGGTGCTACAGGCAGAGCCAT |
| | | GGATGGACCTAGACTGCTGCTGCTCCTGCTGCTTGGAGT |
| | | GTCTCTTGGCGGAGCCAAAGAGGCCTGTCCTACCGGCCT |
| | | GTATACACACTCTGGCGAGTGCTGCAAGGCCTGCAATCT |
| | | TGGAGAAGGCGTGGCACAGCCTTGCGGCGCTAATCAGAC |
| | | AGTGTGCGAGCCTTGCCTGGACAGCGTGACCTTTAGCGA |
| | | CGTGGTGTCTGCCACCGAGCCATGCAAGCCTTGTACCGA |
| | | GTGTGTGGGCCTGCAGAGCATGTCTGCCCCTTGTGTGGA |
| | | AGCCGACGATGCCGTGTGTAGATGCGCCTACGGCTACTA |
| | | CCAGGACGAGACAACAGGCAGATGCGAGGCCTGTAGAG |
| | | TGTGTGAAGCCGGCTCTGGACTGGTGTTCAGCTGCCAAG |
| | | ACAAGCAGAACACCGTGTGCGAGGAATGCCCCGATGGC |
| | | ACCTATAGCGACGAGGCCAACCATGTGGATCCCTGCCTG |
| | | CCTTGTACTGTGTGCGAAGATACCGAGCGGCAGCTGCGC |
| | | GAGTGTACAAGATGGGCTGATGCCGAGTGCGAAGAGAT |
| | | CCCCGGCAGATGGATCACCAGAAGCACACCTCCAGAGG |
| | | GCAGCGATAGCACAGCCCCTTCTACACAAGAGCCCGAG |
| | | GCTCCTCCTGAGCAGGATCTGATTGCCTCTACAGTGGCC |
| | | GGCGTGGTCACAACAGTGATGGGATCTTCTCAGCCCGTG |
| | | GTCACCAGAGGCACCACCGACAATCTGATCCCCGTGTAC |
| | | TGTAGCATCCTGGCCGCCGTGGTTGTGGGACTCGTGGCC |
| | | TATATCGCCTTCAAGAGATGATAA |
| 57 | PL1179 | MARPHPWWLCVLGTLVGLSATPAPKSCPERHYWAQGKLC |
| | (amino acid) | CQMCEPGTFLVKDCDQHRKAAQCDPCIPGVSFSPDHHTRP |
| | | HCESCRHCNSGLLVRNCTITANAECACRNGWQCRDKECTE |
| | | CDPLPNPSLTARSSQALSPHPQPTHLPYVSEMLEARTAGHM |
| | | QTLADFRQLPARTLSTHWPPQRSLCSSDFIRILVIFSGMFLVF |
| | | TLAGALFLHQRRKYRSNKGESPVEPAEPCHYSCPREEEGSTI |
| | | PIQEDYRKPEPACSPKRGRKKLLYIFKQPFMRPVQTTQEED |
| | | GCSCRFPEEEEGGCELRVKFSRSADAPAYQQGQNQLYNEL |
| | | NLGRREEYDVLDKRRGRDPEMGGKPQRRKNPQEGLYNEL |
| | | QKDKMAEAYSEIGMKGERRRGKGHDGLYQGLSTATKDTY |
| | | DALHMQALPPRGSGATNFSLLKQAGDVEENPGPMGRGLLR |
| | | GLWPLHIVLWTRIASTIPPHVQKSVNNDMIVTDNNGAVKFP |
| | | QLCKFCDVRFSTCDNQKSCMSNCSITSICEKPQEVCVAVWR |
| | | KNDENITLETVCHDPKLPYHDFILEDAASPKCIMKEKKKPG |
| | | ETFFMCSCSSDECNDNIIFSEEYNTSNPDLLLVIFQVTGISLLP |
| | | PLGVAISVIIIFYCYRVNRQQKLSS |
| 58 | PL1179 | ATGGCCAGACCTCATCCTTGGTGGCTGTGTGTGCTGGGC |
| | (nucleotide) | ACACTCGTTGGCCTGTCTGCTACACCAGCTCCTAAGAGC |
| | | TGCCCCGAGAGACACTATTGGGCCCAGGGCAAGCTGTGC |
| | | TGCCAGATGTGTGAACCTGGCACCTTCCTGGTCAAGGAC |
| | | TGCGACCAGCACAGAAAGGCCGCTCAGTGCGATCCTTGT |
| | | ATCCCCGGCGTGTCCTTCTCTCCCGACCACCACACAAGA |
| | | CCTCACTGCGAGAGCTGCAGACACTGCAATTCTGGACTG |
| | | CTCGTGCGGAACTGCACCATCACAGCCAATGCCGAGTGC |
| | | GCCTGCAGAAATGGCTGGCAGTGCCGGGACAAAGAATG |
| | | TACCGAGTGCGACCCTCTGCCTAATCCTAGCCTGACCGC |
| | | CAGAAGCAGCCAGGCTTTGTCTCCTCATCCTCAGCCTAC |
| | | ACATCTGCCCTACGTGTCCGAGATGCTGGAAGCCAGAAC |
| | | AGCCGGCCATATGCAGACCCTGGCCGACTTTAGACAGCT |
| | | GCCCGCCAGAACACTGAGCACCCATTGGCCTCCACAGAG |
| | | AAGCCTGTGCAGCAGCGACTTCATCCGGATCCTCGTGAT |
| | | CTTCAGCGGCATGTTCCTGGTGTTCACACTGGCTGGCGCC |
| | | CTGTTTCTGCACCAGCGGAGAAAGTACAGAAGCAACAA |
| | | GGGCGAGAGCCCCGTGGAACCTGCCGAGCCTTGTCACTA |
| | | CAGCTGTCCCAGAGAGGAAGAGGGCAGCACAATCCCCA |
| | | TCCAAGAGGACTACAGAAAGCCCGAGCCTGCCTGCTCTC |
| | | CTAAACGGGGCAGAAAGAAACTCCTGTATATATTCAAAC |
| | | AACCATTTATGAGACCAGTACAAACTACTCAAGAGGAAG |
| | | ATGGCTGTAGCTGCCGATTTCCAGAAGAAGAAGAAGGA |
| | | GGATGTGAACTGAGAGTGAAGTTCAGCAGGAGCGCAGA |
| | | CGCCCCCGCGTACCAGCAGGGCCAGAACCAGCTCTATAA |
| | | CGAGCTCAATCTAGGACGAAGAGAGGAGTACGATGTTTT |
| | | GGACAAGAGACGTGGCCGGGACCCTGAGATGGGGGGAA |
| | | AGCCGCAGAGAAGGAAGAACCCTCAGGAAGGCCTGTAC |
| | | AATGAACTGCAGAAAGATAAGATGGCGGAGGCCTACAG |
| | | TGAGATTGGGATGAAAGGCGAGCGCCGGAGGGGCAAGG |
| | | GGCACGATGGCCTTTACCAGGGTCTCAGTACAGCCACCA |
| | | AGGACACCTACGACGCCCTTCACATGCAGGCCCTGCCCC |
| | | CTCGCGGCAGCGGGGCGACAAATTTCAGCCTTCTGAAAC |
| | | AAGCAGGCGACGTGGAAGAAAACCCCGGTCCAatgggtcggg |
| | | ggctgctcaggggcctgtggccgctgcacatcgtcctgtggacgcgtatcgccagcacgatc |
| | | ccaccgcacgttcagaagtcggttaataacgacatgatagtcactgacaacaacggtgcagtc |
| | | aagtttccacaactgtgtaaattttgtgatgtgagattttccacctgtgacaaccagaaatcctgca |
| | | tgagcaactgcagcatcacctccatctgtgagaagccacaggaagtctgtgtggctgtatgga |
| | | gaaagaatgacgagaacataacactagagacagtttgccatgaccccaagctcccctaccat |
| | | gactttattctggaagatgctgcttctccaaagtgcattatgaaggaaaaaaaaaagcctggtga |
| | | gactttcttcatgtgttcctgtagctctgatgagtgcaatgacaacatcatcttctcagaagaatat |
| | | aacaccagcaatcctgacttgttgctagtcatatttcaagtgacaggcatcagcctcctgccacc |
| | | actgggagttgccatatctgtcatcatcatcttctactgctaccgcgttaaccggcagcagaagc |
| | | tgagttcatagtaa |
| 59 | PL1099 | MARPHPWWLCVLGTLVGLSATPAPKSCPERHYWAQGKLC |
| | (amino acid) | CQMCEPGTFLVKDCDQHRKAAQCDPCIPGVSFSPDHHTRP |
| | | HCESCRHCNSGLLVRNCTITANAECACRNGWQCRDKECTE |
| | | CDPLPNPSLTARSSQALSPHPQPTHLPYVSEMLEARTAGHM |
| | | QTLADFRQLPARTLSTHWPPQRSLCSSDFIRILVIFSGMFLVF |
| | | TLAGALFLHQRRKYRSNKGESPVEPAEPCHYSCPREEEGSTI |
| | | PIQEDYRKPEPACSPKRGRKKLLYIFKQPFMRPVQTTQEED |
| | | GCSCRFPEEEEGGCELRVKFSRSADAPAYQQGQNQLYNEL |
| | | NLGRREEYDVLDKRRGRDPEMGGKPQRRKNPQEGLYNEL |
| | | QKDKMAEAYSEIGMKGERRRGKGHDGLYQGLSTATKDTY |
| | | DALHMQALPPRGSGATNFSLLKQAGDVEENPGPMGAGAT |
| | | GRAMDGPRLLLLLLLGVSLGGAKEACPTGLYTHSGECCKA |
| | | CNLGEGVAQPCGANQTVCEPCLDSVTFSDVVSATEPCKPC |
| | | TECVGLQSMSAPCVEADDAVCRCAYGYYQDETTGRCEAC |
| | | RVCEAGSGLVFSCQDKQNTVCEECPDGTYSDEANHVDPCL |
| | | PCTVCEDTERQLRECTRWADAECEEIPGRWITRSTPPEGSDS |
| | | TAPSTQEPEAPPEQDLIASTVAGVVTTVMGSSQPVVTRGTT |
| | | DNLIPVYCSILAAVVVGLVAYIAFKR |
| 60 | PL1099 | ATGGCCAGACCTCATCCTTGGTGGCTGTGTGTGCTGGGC |
| | (nucleotide) | ACACTCGTTGGCCTGTCTGCTACACCAGCTCCTAAGAGC |
| | | TGCCCCGAGAGACACTATTGGGCCCAGGGCAAGCTGTGC |
| | | TGCCAGATGTGTGAACCTGGCACCTTCCTGGTCAAGGAC |
| | | TGCGACCAGCACAGAAAGGCCGCTCAGTGCGATCCTTGT |
| | | ATCCCCGGCGTGTCCTTCTCTCCCGACCACCACACAAGA |
| | | CCTCACTGCGAGAGCTGCAGACACTGCAATTCTGGACTG |
| | | CTCGTGCGGAACTGCACCATCACAGCCAATGCCGAGTGC |
| | | GCCTGCAGAAATGGCTGGCAGTGCCGGGACAAAGAATG |
| | | TACCGAGTGCGACCCTCTGCCTAATCCTAGCCTGACCGC |
| | | CAGAAGCAGCCAGGCTTTGTCTCCTCATCCTCAGCCTAC |
| | | ACATCTGCCCTACGTGTCCGAGATGCTGGAAGCCAGAAC |
| | | AGCCGGCCATATGCAGACCCTGGCCGACTTTAGACAGCT |
| | | GCCCGCCAGAACACTGAGCACCCATTGGCCTCCACAGAG |
| | | AAGCCTGTGCAGCAGCGACTTCATCCGGATCCTCGTGAT |
| | | CTTCAGCGGCATGTTCCTGGTGTTCACACTGGCTGGCGCC |
| | | CTGTTTCTGCACCAGCGGAGAAAGTACAGAAGCAACAA |
| | | GGGCGAGAGCCCCGTGGAACCTGCCGAGCCTTGTCACTA |
| | | CAGCTGTCCCAGAGAGGAAGAGGGCAGCACAATCCCCA |
| | | TCCAAGAGGACTACAGAAAGCCCGAGCCTGCCTGCTCTC |
| | | CTAAACGGGGCAGAAAGAAACTCCTGTATATATTCAAAC |
| | | AACCATTTATGAGACCAGTACAAACTACTCAAGAGGAAG |
| | | ATGGCTGTAGCTGCCGATTTCCAGAAGAAGAAGAAGGA |
| | | GGATGTGAACTGAGAGTGAAGTTCAGCAGGAGCGCAGA |
| | | CGCCCCCGCGTACCAGCAGGGCCAGAACCAGCTCTATAA |
| | | CGAGCTCAATCTAGGACGAAGAGAGGAGTACGATGTTTT |
| | | GGACAAGAGACGTGGCCGGGACCCTGAGATGGGGGGAA |
| | | AGCCGCAGAGAAGGAAGAACCCTCAGGAAGGCCTGTAC |
| | | AATGAACTGCAGAAAGATAAGATGGCGGAGGCCTACAG |
| | | TGAGATTGGGATGAAAGGCGAGCGCCGGAGGGGCAAGG |
| | | GGCACGATGGCCTTTACCAGGGTCTCAGTACAGCCACCA |
| | | AGGACACCTACGACGCCCTTCACATGCAGGCCCTGCCCC |
| | | CTCGCGGCAGCGGGGCGACAAATTTCAGCCTTCTGAAAC |
| | | AAGCAGGCGACGTGGAAGAAAACCCCGGTCCAATGGGA |
| | | GCTGGTGCTACAGGCAGAGCCATGGATGGACCTAGACTG |
| | | CTGCTGCTCCTGCTGCTTGGAGTGTCTCTTGGCGGAGCCA |
| | | AAGAGGCCTGTCCTACCGGCCTGTATACACACTCTGGCG |
| | | AGTGCTGCAAGGCCTGCAATCTTGGAGAAGGCGTGGCAC |
| | | AGCCTTGCGGCGCTAATCAGACAGTGTGCGAGCCTTGCC |
| | | TGGACAGCGTGACCTTTAGCGACGTGGTGTCTGCCACCG |
| | | AGCCATGCAAGCCTTGTACCGAGTGTGTGGGCCTGCAGA |
| | | GCATGTCTGCCCCTTGTGTGGAAGCCGACGATGCCGTGT |
| | | GTAGATGCGCCTACGGCTACTACCAGGACGAGACAACA |
| | | GGCAGATGCGAGGCCTGTAGAGTGTGTGAAGCCGGCTCT |
| | | GGACTGGTGTTCAGCTGCCAAGACAAGCAGAACACCGTG |
| | | TGCGAGGAATGCCCCGATGGCACCTATAGCGACGAGGCC |
| | | AACCATGTGGATCCCTGCCTGCCTTGTACTGTGTGCGAA |
| | | GATACCGAGCGGCAGCTGCGCGAGTGTACAAGATGGGC |
| | | TGATGCCGAGTGCGAAGAGATCCCCGGCAGATGGATCAC |
| | | CAGAAGCACACCTCCAGAGGGCAGCGATAGCACAGCCC |
| | | CTTCTACACAAGAGCCCGAGGCTCCTCCTGAGCAGGATC |
| | | TGATTGCCTCTACAGTGGCCGGCGTGGTCACAACAGTGA |
| | | TGGGATCTTCTCAGCCCGTGGTCACCAGAGGCACCACCG |
| | | ACAATCTGATCCCCGTGTACTGTAGCATCCTGGCCGCCG |
| | | TGGTTGTGGGACTCGTGGCCTATATCGCCTTCAAGAGAT |
| | | GATAA |
| 61 | PL1092 | MARPHPWWLCVLGTLVGLSATPAPKSCPERHYWAQGKLC |
| | (amino acid) | CQMCEPGTFLVKDCDQHRKAAQCDPCIPGVSFSPDHHTWP |
| | | HCESCRHCNSGLLVRNCTITANAECACRNGWQCRDKECTE |
| | | CDPLPNPSLTARSSQALSPHPQPTHLPYVSEMLEARTAGHM |
| | | QTLADFRQLPARTLSTHWPPQRSLCSSDFIRILVIFSGMFLVF |
| | | TLAGALFLHQRRKYRSNKGESPVEPAEPCHYSCPREEEGSTI |
| | | PIQEDYRKPEPACSPKRGRKKLLYIFKQPFMRPVQTTQEED |
| | | GCSCRFPEEEEGGCELRVKFSRSADAPAYQQGQNQLYNEL |
| | | NLGRREEYDVLDKRRGRDPEMGGKPQRRKNPQEGLYNEL |
| | | QKDKMAEAYSEIGMKGERRRGKGHDGLYQGLSTATKDTY |
| | | DALHMQALPPRRAKRSGSGATNFSLLKQAGDVEENPGPML |
| | | LLVTSLLLCELPHPAFLLIPRKVCNGIGIGEFKDSLSINATNIK |
| | | HFKNCTSISGDLHILPVAFRGDSFTHTPPLDPQELDILKTVKE |
| | | ITGFLLIQAWPENRTDLHAFENLEIIRGRTKQHGQFSLAVVS |
| | | LNITSLGLRSLKEISDGDVIISGNKNLCYANTINWKKLFGTS |
| | | GQKTKIISNRGENSCKATGQVCHALCSPEGCWGPEPRDCVS |
| | | CRNVSRGRECVDKCNLLEGEPREFVENSECIQCHPECLPQA |
| | | MNITCTGRGPDNCIQCAHYIDGPHCVKTCPAGVMGENNTL |
| | | VWKYADAGHVCHLCHPNCTYGCTGPGLEGCPTNGPKIPSI |
| | | ATGMVGALLLLLVVALGIGLFM |
| 62 | PL1092 | ATGGCCAGACCTCATCCTTGGTGGCTGTGTGTGCTGGGC |
| | (nucleotide) | ACACTCGTTGGCCTGTCTGCTACACCAGCTCCTAAGAGC |
| | | TGCCCCGAGAGACACTATTGGGCCCAGGGCAAGCTGTGC |
| | | TGCCAGATGTGTGAACCTGGCACCTTCCTGGTCAAGGAC |
| | | TGCGACCAGCACAGAAAGGCCGCTCAGTGCGATCCTTGT |
| | | ATCCCCGGCGTGTCCTTCTCTCCCGACCACCACACATGG |
| | | CCTCACTGCGAGAGCTGCAGACACTGCAATTCTGGACTG |
| | | CTCGTGCGGAACTGCACCATCACAGCCAATGCCGAGTGC |
| | | GCCTGCAGAAATGGCTGGCAGTGCCGGGACAAAGAATG |
| | | TACCGAGTGCGACCCTCTGCCTAATCCTAGCCTGACCGC |
| | | CAGAAGCAGCCAGGCTTTGTCTCCTCATCCTCAGCCTAC |
| | | ACATCTGCCCTACGTGTCCGAGATGCTGGAAGCCAGAAC |
| | | AGCCGGCCATATGCAGACCCTGGCCGACTTTAGACAGCT |
| | | GCCCGCCAGAACACTGAGCACCCATTGGCCTCCACAGAG |
| | | AAGCCTGTGCAGCAGCGACTTCATCCGGATCCTCGTGAT |
| | | CTTCAGCGGCATGTTCCTGGTGTTCACACTGGCTGGCGCC |
| | | CTGTTTCTGCACCAGCGGAGAAAGTACAGAAGCAACAA |
| | | GGGCGAGAGCCCCGTGGAACCTGCCGAGCCTTGTCACTA |
| | | CAGCTGTCCCAGAGAGGAAGAGGGCAGCACAATCCCCA |
| | | TCCAAGAGGACTACAGAAAGCCCGAGCCTGCCTGCTCTC |
| | | CTAAACGGGGCAGAAAGAAACTCCTGTATATATTCAAAC |
| | | AACCATTTATGAGACCAGTACAAACTACTCAAGAGGAAG |
| | | ATGGCTGTAGCTGCCGATTTCCAGAAGAAGAAGAAGGA |
| | | GGATGTGAACTGAGAGTGAAGTTCAGCAGGAGCGCAGA |
| | | CGCCCCCGCGTACCAGCAGGGCCAGAACCAGCTCTATAA |
| | | CGAGCTCAATCTAGGACGAAGAGAGGAGTACGATGTTTT |
| | | GGACAAGAGACGTGGCCGGGACCCTGAGATGGGGGGAA |
| | | AGCCGCAGAGAAGGAAGAACCCTCAGGAAGGCCTGTAC |
| | | AATGAACTGCAGAAAGATAAGATGGCGGAGGCCTACAG |
| | | TGAGATTGGGATGAAAGGCGAGCGCCGGAGGGGCAAGG |
| | | GGCACGATGGCCTTTACCAGGGTCTCAGTACAGCCACCA |
| | | AGGACACCTACGACGCCCTTCACATGCAGGCCCTGCCCC |
| | | CTCGCCGCGCGAAGCGATCAGGCAGCGGGGCGACAAAT |
| | | TTCAGCCTTCTGAAACAAGCAGGCGACGTGGAAGAAAA |
| | | CCCCGGTCCAatgcttctcctggtgacaagccttctgctctgtgagttaccacacccag |
| | | cattcctcctgatcccacgcaaagtgtgtaacggaataggtattggtgaatttaaagactcactct |
| | | ccataaatgctacgaatattaaacacttcaaaaactgcacctccatcagtggcgatctccacatc |
| | | ctgccggtggcatttaggggtgactccttcacacatactcctcctctggatccacaggaactgg |
| | | atattctgaaaaccgtaaaggaaatcacagggtttttgctgattcaggcttggcctgaaaacagg |
| | | acggacctccatgcctttgagaacctagaaatcatacgcggcaggaccaagcaacatggtca |
| | | gttttctcttgcagtcgtcagcctgaacataacatccttgggattacgctccctcaaggagataag |
| | | tgatggagatgtgataatttcaggaaacaaaaatttgtgctatgcaaatacaataaactggaaaa |
| | | aactgtttgggacctccggtcagaaaaccaaaattataagcaacagaggtgaaaacagctgca |
| | | aggccacaggccaggtctgccatgccttgtgctcccccgagggctgctggggcccggagcc |
| | | cagggactgcgtctcttgccggaatgtcagccgaggcagggaatgcgtggacaagtgcaac |
| | | cttctggagggtgagccaagggagtttgtggagaactctgagtgcatacagtgccacccaga |
| | | gtgcctgcctcaggccatgaacatcacctgcacaggacggggaccagacaactgtatccagt |
| | | gtgcccactacattgacggcccccactgcgtcaagacctgcccggcaggagtcatgggaga |
| | | aaacaacaccctggtctggaagtacgcagacgccggccatgtgtgccacctgtgccatccaa |
| | | actgcacctacggatgcactgggccaggtcttgaaggctgtccaacgaatgggcctaagatc |
| | | ccgtccatcgccactgggatggtgggggccctcctcttgctgctggtggtggccctggggatc |
| | | ggcctcttcatgTGATAA |
| 63 | PL1095 | MARPHPWWLCVLGTLVGLSATPAPKSCPERHYWAQGKLC |
| | (amino acid) | CQMCEPGTFLVKDCDQHRKAAQCDPCIPGVSFSPDHHTWP |
| | | HCESCRHCNSGLLVRNCTITANAECACRNGWQCRDKECTE |
| | | CDPLPNPSLTARSSQALSPHPQPTHLPYVSEMLEARTAGHM |
| | | QTLADFRQLPARTLSTHWPPQRSLCSSDFIRIL VIFSGMFLVF |
| | | TLAGALFLHQRRKYRSNKGESPVEPAEPCHYSCPREEEGSTI |
| | | PIQEDYRKPEPACSPKRGRKKLLYIFKQPFMRPVQTTQEED |
| | | GCSCRFPEEEEGGCELRVKFSRSADAPAYQQGQNQLYNEL |
| | | NLGRREEYDVLDKRRGRDPEMGGKPQRRKNPQEGLYNEL |
| | | QKDKMAEAYSEIGMKGERRRGKGHDGLYQGLSTATKDTY |
| | | DALHMQALPPRRAKRSGSGATNFSLLKQAGDVEENPGPM |
| | | VSKGEELFTGVVPILVELDGDVNGHKFSVSGEGEGDATYG |
| | | KLTLKFICTTGKLPVPWPTLVTTLTYGVQCFSRYPDHMKQH |
| | | DFFKSAMPEGYVQERTIFFKDDGNYKTRAEVKFEGDTLVN |
| | | RIELKGIDFKEDGNILGHKLEYNYNSHNVYIMADKQKNGIK |
| | | VNFKIRHNIEDGSVQLADHYQQNTPIGDGPVLLPDNHYLST |
| | | QSALSKDPNEKRDHMVLLEFVTAAGITLGMDELYK |
| 64 | PL1095 | ATGGCCAGACCTCATCCTTGGTGGCTGTGTGTGCTGGGC |
| | (nucleotide) | ACACTCGTTGGCCTGTCTGCTACACCAGCTCCTAAGAGC |
| | | TGCCCCGAGAGACACTATTGGGCCCAGGGCAAGCTGTGC |
| | | TGCCAGATGTGTGAACCTGGCACCTTCCTGGTCAAGGAC |
| | | TGCGACCAGCACAGAAAGGCCGCTCAGTGCGATCCTTGT |
| | | ATCCCCGGCGTGTCCTTCTCTCCCGACCACCACACATGG |
| | | CCTCACTGCGAGAGCTGCAGACACTGCAATTCTGGACTG |
| | | CTCGTGCGGAACTGCACCATCACAGCCAATGCCGAGTGC |
| | | GCCTGCAGAAATGGCTGGCAGTGCCGGGACAAAGAATG |
| | | TACCGAGTGCGACCCTCTGCCTAATCCTAGCCTGACCGC |
| | | CAGAAGCAGCCAGGCTTTGTCTCCTCATCCTCAGCCTAC |
| | | ACATCTGCCCTACGTGTCCGAGATGCTGGAAGCCAGAAC |
| | | AGCCGGCCATATGCAGACCCTGGCCGACTTTAGACAGCT |
| | | GCCCGCCAGAACACTGAGCACCCATTGGCCTCCACAGAG |
| | | AAGCCTGTGCAGCAGCGACTTCATCCGGATCCTCGTGAT |
| | | CTTCAGCGGCATGTTCCTGGTGTTCACACTGGCTGGCGCC |
| | | CTGTTTCTGCACCAGCGGAGAAAGTACAGAAGCAACAA |
| | | GGGCGAGAGCCCCGTGGAACCTGCCGAGCCTTGTCACTA |
| | | CAGCTGTCCCAGAGAGGAAGAGGGCAGCACAATCCCCA |
| | | TCCAAGAGGACTACAGAAAGCCCGAGCCTGCCTGCTCTC |
| | | CTAAACGGGGCAGAAAGAAACTCCTGTATATATTCAAAC |
| | | AACCATTTATGAGACCAGTACAAACTACTCAAGAGGAAG |
| | | ATGGCTGTAGCTGCCGATTTCCAGAAGAAGAAGAAGGA |
| | | GGATGTGAACTGAGAGTGAAGTTCAGCAGGAGCGCAGA |
| | | CGCCCCCGCGTACCAGCAGGGCCAGAACCAGCTCTATAA |
| | | CGAGCTCAATCTAGGACGAAGAGAGGAGTACGATGTTTT |
| | | GGACAAGAGACGTGGCCGGGACCCTGAGATGGGGGGAA |
| | | AGCCGCAGAGAAGGAAGAACCCTCAGGAAGGCCTGTAC |
| | | AATGAACTGCAGAAAGATAAGATGGCGGAGGCCTACAG |
| | | TGAGATTGGGATGAAAGGCGAGCGCCGGAGGGGCAAGG |
| | | GGCACGATGGCCTTTACCAGGGTCTCAGTACAGCCACCA |
| | | AGGACACCTACGACGCCCTTCACATGCAGGCCCTGCCCC |
| | | CTCGCCGCGCGAAGCGATCAGGCAGCGGGGCGACAAAT |
| | | TTCAGCCTTCTGAAACAAGCAGGCGACGTGGAAGAAAA |
| | | CCCCGGTCCAATGGTGAGCAAGGGCGAGGAGCTGTTCAC |
| | | CGGGGTGGTGCCCATCCTGGTCGAGCTGGACGGCGACGT |
| | | AAACGGCCACAAGTTCAGCGTGTCCGGCGAGGGCGAGG |
| | | GCGATGCCACCTACGGCAAGCTGACCCTGAAGTTCATCT |
| | | GCACCACCGGCAAGCTGCCCGTGCCCTGGCCCACCCTCG |
| | | TGACCACCCTGACCTACGGCGTGCAGTGCTTCAGCCGCT |
| | | ACCCCGACCACATGAAGCAGCACGACTTCTTCAAGTCCG |
| | | CCATGCCCGAAGGCTACGTCCAGGAGCGCACCATCTTCT |
| | | TCAAGGACGACGGCAACTACAAGACCCGCGCCGAGGTG |
| | | AAGTTCGAGGGCGACACCCTGGTGAACCGCATCGAGCTG |
| | | AAGGGCATCGACTTCAAGGAGGACGGCAACATCCTGGG |
| | | GCACAAGCTGGAGTACAACTACAACAGCCACAACGTCTA |
| | | TATCATGGCCGACAAGCAGAAGAACGGCATCAAGGTGA |
| | | ACTTCAAGATCCGCCACAACATCGAGGACGGCAGCGTGC |
| | | AGCTCGCCGACCACTACCAGCAGAACACCCCCATCGGCG |
| | | ACGGCCCCGTGCTGCTGCCCGACAACCACTACCTGAGCA |
| | | CCCAGTCCGCCCTGAGCAAAGACCCCAACGAGAAGCGC |
| | | GATCACATGGTCCTGCTGGAGTTCGTGACCGCCGCCGGG |
| | | ATCACTCTCGGCATGGACGAGCTGTACAAGTAA |
| 65 | PL1109 | MARPHPWWLCVLGTLVGLSATPAPKSCPERHYWAQGKLC |
| | (amino acid) | CQMCEPGTFLVKDCDQHRKAAQCDPCIPGVSFSPDHHTWP |
| | | HCESCRHCNSGLLVRNCTITANAECACRNGWQCRDKECTE |
| | | CDPLPNPSLTARSSQALSPHPQPTHLPYVSEMLEARTAGHM |
| | | QTLADFRQLPARTLSTHWPPQRSLCSSDFIRILVIFSGMFLVF |
| | | TLAGALFLHQRRKYRSNKGESPVEPAEPCHYSCPREEEGSTI |
| | | PIQEDYRKPEPACSPKRGRKKLLYIFKQPFMRPVQTTQEED |
| | | GCSCRFPEEEEGGCELRVKFSRSADAPAYQQGQNQLYNEL |
| | | NLGRREEYDVLDKRRGRDPEMGGKPQRRKNPQEGLYNEL |
| | | QKDKMAEAYSEIGMKGERRRGKGHDGLYQGLSTATKDTY |
| | | DALHMQALPPR |
| 66 | PL1109 | ATGGCCAGACCTCATCCTTGGTGGCTGTGTGTGCTGGGC |
| | (nucleotide) | ACACTCGTTGGCCTGTCTGCTACACCAGCTCCTAAGAGC |
| | | TGCCCCGAGAGACACTATTGGGCCCAGGGCAAGCTGTGC |
| | | TGCCAGATGTGTGAACCTGGCACCTTCCTGGTCAAGGAC |
| | | TGCGACCAGCACAGAAAGGCCGCTCAGTGCGATCCTTGT |
| | | ATCCCCGGCGTGTCCTTCTCTCCCGACCACCACACATGG |
| | | CCTCACTGCGAGAGCTGCAGACACTGCAATTCTGGACTG |
| | | CTCGTGCGGAACTGCACCATCACAGCCAATGCCGAGTGC |
| | | GCCTGCAGAAATGGCTGGCAGTGCCGGGACAAAGAATG |
| | | TACCGAGTGCGACCCTCTGCCTAATCCTAGCCTGACCGC |
| | | CAGAAGCAGCCAGGCTTTGTCTCCTCATCCTCAGCCTAC |
| | | ACATCTGCCCTACGTGTCCGAGATGCTGGAAGCCAGAAC |
| | | AGCCGGCCATATGCAGACCCTGGCCGACTTTAGACAGCT |
| | | GCCCGCCAGAACACTGAGCACCCATTGGCCTCCACAGAG |
| | | AAGCCTGTGCAGCAGCGACTTCATCCGGATCCTCGTGAT |
| | | CTTCAGCGGCATGTTCCTGGTGTTCACACTGGCTGGCGCC |
| | | CTGTTTCTGCACCAGCGGAGAAAGTACAGAAGCAACAA |
| | | GGGCGAGAGCCCCGTGGAACCTGCCGAGCCTTGTCACTA |
| | | CAGCTGTCCCAGAGAGGAAGAGGGCAGCACAATCCCCA |
| | | TCCAAGAGGACTACAGAAAGCCCGAGCCTGCCTGCTCTC |
| | | CTAAACGGGGCAGAAAGAAACTCCTGTATATATTCAAAC |
| | | AACCATTTATGAGACCAGTACAAACTACTCAAGAGGAAG |
| | | ATGGCTGTAGCTGCCGATTTCCAGAAGAAGAAGAAGGA |
| | | GGATGTGAACTGAGAGTGAAGTTCAGCAGGAGCGCAGA |
| | | CGCCCCCGCGTACCAGCAGGGCCAGAACCAGCTCTATAA |
| | | CGAGCTCAATCTAGGACGAAGAGAGGAGTACGATGTTTT |
| | | GGACAAGAGACGTGGCCGGGACCCTGAGATGGGGGGAA |
| | | AGCCGCAGAGAAGGAAGAACCCTCAGGAAGGCCTGTAC |
| | | AATGAACTGCAGAAAGATAAGATGGCGGAGGCCTACAG |
| | | TGAGATTGGGATGAAAGGCGAGCGCCGGAGGGGCAAGG |
| | | GGCACGATGGCCTTTACCAGGGTCTCAGTACAGCCACCA |
| | | AGGACACCTACGACGCCCTTCACATGCAGGCCCTGCCCC |
| | | CTCGCTAA |
| 67 | PL1091 | MARPHPWWLCVLGTLVGLSATPAPKSCPERHYWAQGKLC |
| | (amino acid) | CQMCEPGTFLVKDCDQHRKAAQCDPCIPGVSFSPDHHTRP |
| | | HCESCRHCNSGLLVRNCTITANAECACRNGWQCRDKECTE |
| | | CDPLPNPSLTARSSQALSPHPQPTHLPYVSEMLEARTAGHM |
| | | QTLADFRQLPARTLSTHWPPQRSLCSSDFIRILVIFSGMFLVF |
| | | TLAGALFLHKRGRKKLLYIFKQPFMRPVQTTQEEDGCSCRF |
| | | PEEEEGGCELRVKFSRSADAPAYQQGQNQLYNELNLGRRE |
| | | EYDVLDKRRGRDPEMGGKPQRRKNPQEGLYNELQKDKMA |
| | | EAYSEIGMKGERRRGKGHDGLYQGLSTATKDTYDALHMQ |
| | | ALPPRRAKRSGSGATNFSLLKQAGDVEENPGPMLLLVTSLL |
| | | LCELPHPAFLLIPRKVCNGIGIGEFKDSLSINATNIKHFKNCT |
| | | SISGDLHILPVAFRGDSFTHTPPLDPQELDILKTVKEITGFLLI |
| | | QAWPENRTDLHAFENLEIIRGRTKQHGQFSLAVVSLNITSL |
| | | GLRSLKEISDGDVIISGNKNLCYANTINWKKLFGTSGQKTKI |
| | | ISNRGENSCKATGQVCHALCSPEGCWGPEPRDCVSCRNVS |
| | | RGRECVDKCNLLEGEPREFVENSECIQCHPECLPQAMNITC |
| | | TGRGPDNCIQCAHYIDGPHCVKTCPAGVMGENNTLVWKY |
| | | ADAGHVCHLCHPNCTYGCTGPGLEGCPTNGPKIPSIATGM |
| | | VGALLLLLVVALGIGLFM |
| 68 | PL1091 | ATGGCCAGACCTCATCCTTGGTGGCTGTGTGTGCTGGGC |
| | (nucleotide) | ACACTCGTTGGCCTGTCTGCTACACCAGCTCCTAAGAGC |
| | | TGCCCCGAGAGACACTATTGGGCCCAGGGCAAGCTGTGC |
| | | TGCCAGATGTGTGAACCTGGCACCTTCCTGGTCAAGGAC |
| | | TGCGACCAGCACAGAAAGGCCGCTCAGTGCGATCCTTGT |
| | | ATCCCCGGCGTGTCCTTCTCTCCCGACCACCACACAAGA |
| | | CCTCACTGCGAGAGCTGCAGACACTGCAATTCTGGACTG |
| | | CTCGTGCGGAACTGCACCATCACAGCCAATGCCGAGTGC |
| | | GCCTGCAGAAATGGCTGGCAGTGCCGGGACAAAGAATG |
| | | TACCGAGTGCGACCCTCTGCCTAATCCTAGCCTGACCGC |
| | | CAGAAGCAGCCAGGCTTTGTCTCCTCATCCTCAGCCTAC |
| | | ACATCTGCCCTACGTGTCCGAGATGCTGGAAGCCAGAAC |
| | | AGCCGGCCATATGCAGACCCTGGCCGACTTTAGACAGCT |
| | | GCCCGCCAGAACACTGAGCACCCATTGGCCTCCACAGAG |
| | | AAGCCTGTGCAGCAGCGACTTCATCCGGATCCTCGTGAT |
| | | CTTCAGCGGCATGTTCCTGGTGTTCACACTGGCTGGCGCC |
| | | CTGTTTCTGCACAAACGGGGCAGAAAGAAACTCCTGTAT |
| | | ATATTCAAACAACCATTTATGAGACCAGTACAAACTACT |
| | | CAAGAGGAAGATGGCTGTAGCTGCCGATTTCCAGAAGA |
| | | AGAAGAAGGAGGATGTGAACTGAGAGTGAAGTTCAGCA |
| | | GGAGCGCAGACGCCCCCGCGTACCAGCAGGGCCAGAAC |
| | | CAGCTCTATAACGAGCTCAATCTAGGACGAAGAGAGGA |
| | | GTACGATGTTTTGGACAAGAGACGTGGCCGGGACCCTGA |
| | | GATGGGGGGAAAGCCGCAGAGAAGGAAGAACCCTCAGG |
| | | AAGGCCTGTACAATGAACTGCAGAAAGATAAGATGGCG |
| | | GAGGCCTACAGTGAGATTGGGATGAAAGGCGAGCGCCG |
| | | GAGGGGCAAGGGGCACGATGGCCTTTACCAGGGTCTCA |
| | | GTACAGCCACCAAGGACACCTACGACGCCCTTCACATGC |
| | | AGGCCCTGCCCCCTCGCCGCGCGAAGCGATCAGGCAGCG |
| | | GGGCGACAAATTTCAGCCTTCTGAAACAAGCAGGCGACG |
| | | TGGAAGAAAACCCCGGTCCAatgcttctcctggtgacaagccttctgctctgt |
| | | gagttaccacacccagcattcctcctgatcccacgcaaagtgtgtaacggaataggtattggtg |
| | | aatttaaagactcactctccataaatgctacgaatattaaacacttcaaaaactgcacctccatca |
| | | gtggcgatctccacatcctgccggtggcatttaggggtgactccttcacacatactcctcctctg |
| | | gatccacaggaactggatattctgaaaaccgtaaaggaaatcacagggtttttgctgattcagg |
| | | cttggcctgaaaacaggacggacctccatgcctttgagaacctagaaatcatacgcggcagg |
| | | accaagcaacatggtcagttttctcttgcagtcgtcagcctgaacataacatccttgggattacg |
| | | ctccctcaaggagataagtgatggagatgtgataatttcaggaaacaaaaatttgtgctatgcaa |
| | | atacaataaactggaaaaaactgtttgggacctccggtcagaaaaccaaaattataagcaaca |
| | | gaggtgaaaacagctgcaaggccacaggccaggtctgccatgccttgtgctcccccgaggg |
| | | ctgctggggcccggagcccagggactgcgtctcttgccggaatgtcagccgaggcagggaa |
| | | tgcgtggacaagtgcaaccttctggagggtgagccaagggagtttgtggagaactctgagtg |
| | | catacagtgccacccagagtgcctgcctcaggccatgaacatcacctgcacaggacgggga |
| | | ccagacaactgtatccagtgtgcccactacattgacggcccccactgcgtcaagacctgcccg |
| | | gcaggagtcatgggagaaaacaacaccctggtctggaagtacgcagacgccggccatgtgt |
| | | gccacctgtgccatccaaactgcacctacggatgcactgggccaggtcttgaaggctgtccaa |
| | | cgaatgggcctaagatcccgtccatcgccactgggatggtgggggccctcctcttgctgctgg |
| | | tggtggccctggggatcggcctcttcatgTGATAA |
| 69 | PL1094 | MARPHPWWLCVLGTLVGLSATPAPKSCPERHYWAQGKLC |
| | (amino acid) | CQMCEPGTFLVKDCDQHRKAAQCDPCIPGVSFSPDHHTRP |
| | | HCESCRHCNSGLLVRNCTITANAECACRNGWQCRDKECTE |
| | | CDPLPNPSLTARSSQALSPHPQPTHLPYVSEMLEARTAGHM |
| | | QTLADFRQLPARTLSTHWPPQRSLCSSDFIRILVIFSGMFLVF |
| | | TLAGALFLHKRGRKKLLYIFKQPFMRPVQTTQEEDGCSCRF |
| | | PEEEEGGCELRVKFSRSADAPAYQQGQNQLYNELNLGRRE |
| | | EYDVLDKRRGRDPEMGGKPQRRKNPQEGLYNELQKDKMA |
| | | EAYSEIGMKGERRRGKGHDGLYQGLSTATKDTYDALHMQ |
| | | ALPPRRAKRSGSGATNFSLLKQAGDVEENPGPMVSKGEEL |
| | | FTGVVPILVELDGDVNGHKFSVSGEGEGDATYGKLTLKFIC |
| | | TTGKLPVPWPTLVTTLTYGVQCFSRYPDHMKQHDFFKSAM |
| | | PEGYVQERTIFFKDDGNYKTRAEVKFEGDTLVNRIELKGID |
| | | FKEDGNILGHKLEYNYNSHNVYIMADKQKNGIKVNFKIRH |
| | | NIEDGSVQLADHYQQNTPIGDGPVLLPDNHYLSTQSALSKD |
| | | PNEKRDHMVLLEFVTAAGITLGMDELYK |
| 70 | PL1094 | ATGGCCAGACCTCATCCTTGGTGGCTGTGTGTGCTGGGC |
| | (nucleotide) | ACACTCGTTGGCCTGTCTGCTACACCAGCTCCTAAGAGC |
| | | TGCCCCGAGAGACACTATTGGGCCCAGGGCAAGCTGTGC |
| | | TGCCAGATGTGTGAACCTGGCACCTTCCTGGTCAAGGAC |
| | | TGCGACCAGCACAGAAAGGCCGCTCAGTGCGATCCTTGT |
| | | ATCCCCGGCGTGTCCTTCTCTCCCGACCACCACACAAGA |
| | | CCTCACTGCGAGAGCTGCAGACACTGCAATTCTGGACTG |
| | | CTCGTGCGGAACTGCACCATCACAGCCAATGCCGAGTGC |
| | | GCCTGCAGAAATGGCTGGCAGTGCCGGGACAAAGAATG |
| | | TACCGAGTGCGACCCTCTGCCTAATCCTAGCCTGACCGC |
| | | CAGAAGCAGCCAGGCTTTGTCTCCTCATCCTCAGCCTAC |
| | | ACATCTGCCCTACGTGTCCGAGATGCTGGAAGCCAGAAC |
| | | AGCCGGCCATATGCAGACCCTGGCCGACTTTAGACAGCT |
| | | GCCCGCCAGAACACTGAGCACCCATTGGCCTCCACAGAG |
| | | AAGCCTGTGCAGCAGCGACTTCATCCGGATCCTCGTGAT |
| | | CTTCAGCGGCATGTTCCTGGTGTTCACACTGGCTGGCGCC |
| | | CTGTTTCTGCACAAACGGGGCAGAAAGAAACTCCTGTAT |
| | | ATATTCAAACAACCATTTATGAGACCAGTACAAACTACT |
| | | CAAGAGGAAGATGGCTGTAGCTGCCGATTTCCAGAAGA |
| | | AGAAGAAGGAGGATGTGAACTGAGAGTGAAGTTCAGCA |
| | | GGAGCGCAGACGCCCCCGCGTACCAGCAGGGCCAGAAC |
| | | CAGCTCTATAACGAGCTCAATCTAGGACGAAGAGAGGA |
| | | GTACGATGTTTTGGACAAGAGACGTGGCCGGGACCCTGA |
| | | GATGGGGGGAAAGCCGCAGAGAAGGAAGAACCCTCAGG |
| | | AAGGCCTGTACAATGAACTGCAGAAAGATAAGATGGCG |
| | | GAGGCCTACAGTGAGATTGGGATGAAAGGCGAGCGCCG |
| | | GAGGGGCAAGGGGCACGATGGCCTTTACCAGGGTCTCA |
| | | GTACAGCCACCAAGGACACCTACGACGCCCTTCACATGC |
| | | AGGCCCTGCCCCCTCGCCGCGCGAAGCGATCAGGCAGCG |
| | | GGGCGACAAATTTCAGCCTTCTGAAACAAGCAGGCGACG |
| | | TGGAAGAAAACCCCGGTCCAATGGTGAGCAAGGGCGAG |
| | | GAGCTGTTCACCGGGGTGGTGCCCATCCTGGTCGAGCTG |
| | | GACGGCGACGTAAACGGCCACAAGTTCAGCGTGTCCGGC |
| | | GAGGGCGAGGGCGATGCCACCTACGGCAAGCTGACCCT |
| | | GAAGTTCATCTGCACCACCGGCAAGCTGCCCGTGCCCTG |
| | | GCCCACCCTCGTGACCACCCTGACCTACGGCGTGCAGTG |
| | | CTTCAGCCGCTACCCCGACCACATGAAGCAGCACGACTT |
| | | CTTCAAGTCCGCCATGCCCGAAGGCTACGTCCAGGAGCG |
| | | CACCATCTTCTTCAAGGACGACGGCAACTACAAGACCCG |
| | | CGCCGAGGTGAAGTTCGAGGGCGACACCCTGGTGAACC |
| | | GCATCGAGCTGAAGGGCATCGACTTCAAGGAGGACGGC |
| | | AACATCCTGGGGCACAAGCTGGAGTACAACTACAACAG |
| | | CCACAACGTCTATATCATGGCCGACAAGCAGAAGAACG |
| | | GCATCAAGGTGAACTTCAAGATCCGCCACAACATCGAGG |
| | | ACGGCAGCGTGCAGCTCGCCGACCACTACCAGCAGAAC |
| | | ACCCCCATCGGCGACGGCCCCGTGCTGCTGCCCGACAAC |
| | | CACTACCTGAGCACCCAGTCCGCCCTGAGCAAAGACCCC |
| | | AACGAGAAGCGCGATCACATGGTCCTGCTGGAGTTCGTG |
| | | ACCGCCGCCGGGATCACTCTCGGCATGGACGAGCTGTAC |
| | | AAGTAA |
| 71 | PL1108 | MARPHPWWLCVLGTLVGLSATPAPKSCPERHYWAQGKLC |
| | (amino acid) | CQMCEPGTFLVKDCDQHRKAAQCDPCIPGVSFSPDHHTRP |
| | | HCESCRHCNSGLLVRNCTITANAECACRNGWQCRDKECTE |
| | | CDPLPNPSLTARSSQALSPHPQPTHLPYVSEMLEARTAGHM |
| | | QTLADFRQLPARTLSTHWPPQRSLCSSDFIRILVIFSGMFLVF |
| | | TLAGALFLHKRGRKKLLYIFKQPFMRPVQTTQEEDGCSCRF |
| | | PEEEEGGCELRVKFSRSADAPAYQQGQNQLYNELNLGRRE |
| | | EYDVLDKRRGRDPEMGGKPQRRKNPQEGLYNELQKDKMA |
| | | EAYSEIGMKGERRRGKGHDGLYQGLSTATKDTYDALHMQ |
| | | ALPPR |
| 72 | PL1108 | ATGGCCAGACCTCATCCTTGGTGGCTGTGTGTGCTGGGC |
| | (nucleotide) | ACACTCGTTGGCCTGTCTGCTACACCAGCTCCTAAGAGC |
| | | TGCCCCGAGAGACACTATTGGGCCCAGGGCAAGCTGTGC |
| | | TGCCAGATGTGTGAACCTGGCACCTTCCTGGTCAAGGAC |
| | | TGCGACCAGCACAGAAAGGCCGCTCAGTGCGATCCTTGT |
| | | ATCCCCGGCGTGTCCTTCTCTCCCGACCACCACACAAGA |
| | | CCTCACTGCGAGAGCTGCAGACACTGCAATTCTGGACTG |
| | | CTCGTGCGGAACTGCACCATCACAGCCAATGCCGAGTGC |
| | | GCCTGCAGAAATGGCTGGCAGTGCCGGGACAAAGAATG |
| | | TACCGAGTGCGACCCTCTGCCTAATCCTAGCCTGACCGC |
| | | CAGAAGCAGCCAGGCTTTGTCTCCTCATCCTCAGCCTAC |
| | | ACATCTGCCCTACGTGTCCGAGATGCTGGAAGCCAGAAC |
| | | AGCCGGCCATATGCAGACCCTGGCCGACTTTAGACAGCT |
| | | GCCCGCCAGAACACTGAGCACCCATTGGCCTCCACAGAG |
| | | AAGCCTGTGCAGCAGCGACTTCATCCGGATCCTCGTGAT |
| | | CTTCAGCGGCATGTTCCTGGTGTTCACACTGGCTGGCGCC |
| | | CTGTTTCTGCACAAACGGGGCAGAAAGAAACTCCTGTAT |
| | | ATATTCAAACAACCATTTATGAGACCAGTACAAACTACT |
| | | CAAGAGGAAGATGGCTGTAGCTGCCGATTTCCAGAAGA |
| | | AGAAGAAGGAGGATGTGAACTGAGAGTGAAGTTCAGCA |
| | | GGAGCGCAGACGCCCCCGCGTACCAGCAGGGCCAGAAC |
| | | CAGCTCTATAACGAGCTCAATCTAGGACGAAGAGAGGA |
| | | GTACGATGTTTTGGACAAGAGACGTGGCCGGGACCCTGA |
| | | GATGGGGGGAAAGCCGCAGAGAAGGAAGAACCCTCAGG |
| | | AAGGCCTGTACAATGAACTGCAGAAAGATAAGATGGCG |
| | | GAGGCCTACAGTGAGATTGGGATGAAAGGCGAGCGCCG |
| | | GAGGGGCAAGGGGCACGATGGCCTTTACCAGGGTCTCA |
| | | GTACAGCCACCAAGGACACCTACGACGCCCTTCACATGC |
| | | AGGCCCTGCCCCCTCGCTAA |
| 73 | CD28 | FWVLVVVGGVLACYSLLVTVAFIIFWVRSKRSRLLHSDYM |
| | costimulatory | NMTPRRPGPTRKHYQPYAPPRDFAAYRS |
| | domain | |
| | (amino acid) | |
| 74 | CD28 | FWVRSKRSRLLHSDYMNMTPRRPGPTRKHYQPYAPPRDFA |
| | costimulatory | AYRS |
| | domain | |
| | (amino acid) | |
| 75 | ICOS | TKKKYSSSVHDPNGEYMFMRAVNTAKKSRLTDVTL |
| | costimulatory | |
| | domain | |
| | (amino acid) | |
| 76 | OX40 | RRDQRLPPDAHKPPGGGSFRTPIQEEQADAHSTLAKI |
| | (amino acid) | |
| 77 | P2A cleavage | SGSGATNFSLLKQAGDVEENPGP |
| | motif-2 | |
| | (amino acid) | |
| 78 | P2A cleavage | ATNFSLLKQAGDVEENPGP |
| | motif-3 | |
| | (amino acid) | |
| 79 | Furin cleavage | RAKR |
| | site | |
| | (amino acid) | |
| 80 | F2A cleavage | VKQTLNNFDLLKLAGDVESNPGP |
| | motif | |
| | (amino acid) | |
| 81 | E2A cleavage | QCTNYALLKLAGDVESNPGP |
| | motif | |
| | (amino acid) | |
| 82 | T2A cleavage | EGRSLLTCGDVEENPGP |
| | motif | |
| | (amino acid) | |
| 83 | IRES 1 | CTAACGTTACTGGCCGAAGCCGCTTGGAATAAGGCCGGTGT |
| | nucleotide | GCGTTTGTCTATATGTTATTTTCCACCATATTGCCGTCTTTTG |
| | | GCAATGTGAGGGCCCGGAAACCTGGCCCTGTCTTCTTGACGA |
| | | GCATTCCTAGGGGTCTTTCCCCTCTCGCCAAAGGAATGCAAG |
| | | GTCTGTTGAATGTCGTGAAGGAAGCAGTTCCTCTGGAAGCTT |
| | | CTTGAAGACAAACAACGTCTGTAGCGACCCTTTGCAGGCAG |
| | | CGGAACCCCCCACCTGGCGACAGGTGCCTCTGCGGCCAAAA |
| | | GCCACGTGTATAAGATACACCTGCAAAGGCGGCACAACCCC |
| | | AGTGCCAC |
| | | GTTGTGAGTTGGATAGTTGTGGAAAGAGTCAAATGGCTCTCC |
| | | TCAAGCGTATTCAACAAGGGGCTGAAGGATGCCCAGAAGGT |
| | | ACCCCATTGTATGGGATCTGATCTGGGGCCTCGGTGCACATG |
| | | CTTTACATGTGTTTAGTCGAGGTTAAAAAAACGTCTAGGCCC |
| | | CCCGAACCACGGGGACGTGGTTTTCCTTTGAAAAACACGAT |
| | | GATA |
| 84 | IRES 2 | AGCAGGTTTCCCCAACTGACACAAAACGTGCAACTTGAAAC |
| | nucleotide | TCCGCCTGGTCTTTCCAGGTCTAGAGGGGTAACACTTTGTAC |
| | | TGCGTTTGGCTCCACGCTCGATCCACTGGCGAGTGTTAGTAA |
| | | CAGCACTGTTGCTTCGTAGCGGAGCATGACGGCCGTGGGAA |
| | | CTCCTCCTTGGTAACAAGGACCCACGGGGCCAAAAGCCACG |
| | | CCCACACGGGCCCGTCATGTGTGCAACCCCAGCACGGCGAC |
| | | TTTACTGCGAAACCCACTTTAAAGTGACATTGAAACTGGTAC |
| | | CCACACACTGGTGACAGGCTAAGGATGCCCTTCAGGTACCC |
| | | CGAGGTAACACGCGACACTCGGGATCTGAGAAGGGGACTGG |
| | | GGCTTCTATAAAAGCGCTCGGTTTAAAAAGCTTCTATGCCTG |
| | | AATAGGTGACCGGAGGTCGGCACCTTTCCTTTGCAATT |
| | | ACTGACCAC |
| 85 | Signal Peptide | MALPVTALLLPLALLLHAARP |
| | (amino acid) | |
| 86 | SIL-15 | NWVNVISDLKKIEDLIQSMHIDATLYTESDVHPSCKVTAMK |
| | | CFLLELQVISLESGDASIHDTVENLIILANNSLSSNGNVTESG |
| | | CKECEELEEKNIKEFLQSFVHIVQMFINTS |
| 87 | CISH-50608551 | ggacugggcagcggccccug |
| | (guide RNA) | |
| 88 | CISH-50608572 | guccuuugcuggcuguggag |
| | (guide RNA) | |
| 89 | CISH-50608560 | cuguggagcggacugggcag |
| | (guide RNA) | |
| 90 | CISH + | uccacagccagcaaaggacg |
| | 50608587 | |
| | (guide RNA) | |
| 91 | Full length | MARPHPWWLCVLGTLVGLSATPAPKSCPERHYWAQGKLC |
| | CD27 + R58W | CQMCEPGTFLVKDCDQHRKAAQCDPCIPGVSFSPDHHTWP |
| | (amino acid) | HCESCRHCNSGLLVRNCTITANAECACRNGWQCRDKECTE |
| | | CDPLPNPSLTARSSQALSPHPQPTHLPYVSEMLEARTAGHM |
| | | QTLADFRQLPARTLSTHWPPQRSLCSSDFIRILVIFSGMFLVF |
| | | TLAGALFLHQRRKYRSNKGESPVEPAEPCHYSCPREEEGSTI |
| | | PIQEDYRKPEPACSP |
| 92 | Truncated | MARPHPWWLCVLGTLVGLSATPAPKSCPERHYWAQGKLC |
| | CD27 + R58W | CQMCEPGTFLVKDCDQHRKAAQCDPCIPGVSFSPDHHTWP |
| | (amino acid) | HCESCRHCNSGLLVRNCTITANAECACRNGWQCRDKECTE |
| | | CDPLPNPSLTARSSQALSPHPQPTHLPYVSEMLEARTAGHM |
| | | QTLADFRQLPARTLSTHWPPQRSLCSSDFIRILVIFSGMFLVF |
| | | TLAGALFLH |
| 93 | Guide RNA for | AAGACUCUUUAAAGAAGGCA |
| | disrupting CBL- | |
| | B | |
| 94 | Guide RNA for | AGUACUCAUUCUCACUGAGU |
| | disrupting CBL- | |
| | B | |
| 95 | Guide RNA for | CGUAAAUGCUGAUAUGUAUC |
| | disrupting CBL- | |
| | B | |
| 96 | CAS9 CISH | TGTACAGCAGTGGCTGGTGG(AGG) |
| | sgRNA | |
| 97 | MAD7 CISH | (TTTA)GGTGTACAGCAGTGGCTGGTG |
| | sgRNA | |
| 98 | sgRNA | TAATCTGGTGGACCTCATGA(AGG) |
| 99 | Guide RNA | AAGCCCAUCAGUUUGGGUUG |
| 100 | Guide RNA | UGUACAAGCUCCACAAUGGA |
| 101 | Guide RNA | CAAAUGGGCAAGCCUUGCGG |
| 102 | sgRNA | CCTGAATAAACTCCACCGCA(AGG) |
| 103 | CAS9 ICAM-1 | AAAGUCAUCCUGCCCCGGGG |
| | sgRNA | |
| 104 | MAD7 ICAM-1 | TTTG/AATAGCACATTGGTTGGCTAT |
| | sgRNA | |
| 105 | CAS9 CD58 | AAAUAUAUGGUGUUGUGUAU |
| | sgRNA | |
| 106 | MAD7 CD58 | TTTA/GACACTGTGTCAGGTAGCCTC |
| | sgRNA | |

What is claimed is:

1. A γδ T cell comprising:
a chimeric antigen receptor (CAR), wherein the CAR comprises a binding domain that recognizes the CD70 antigen, at least one costimulatory domain, and at least one intracellular signaling domain; and
wherein the γδ T cell functionally expresses the binding domain of the CAR on the surface of the γδ T cell.

2. The γδ T cell of claim 1, wherein the binding domain comprises a full-length CD27 receptor having an amino acid sequence as set forth in SEQ ID NO: 33.

3. The γδ T cell of claim 1, wherein the binding domain comprises a full-length CD27 receptor having an amino acid sequence as set forth in SEQ ID NO: 91.

4. The γδ T cell of any one of claims 1-3, wherein the at least one costimulatory domain is selected from TLR1, TLR2, TLR3, TLR4, TLR5, TLR6, TLR7, TLR8, TLR9, TLR10, CARD11, CD2, CD3C, CD7, CD27, CD28, CD30, CD40, CD54 (ICAM), CD70, CD80, CD83, CD86, CD134 (OX40), CD137 (4-1BB), CD278 (ICOS), FcR, LAT, NKD2C, SLP76, TRIM, and ZAP70, or combinations thereof.

5. The γδ T cell of claim 4, wherein the at least one costimulatory domain is 4-1BB.

6. The γδ T cell of any one of claims 1-5, wherein the at least one intracellular signaling domain is selected from CD3ζ, DAP12, LFA-1, and repeat (2-5) DAP10 YINM motifs.

7. The γδ T cell of claim 6, wherein the at least one intracellular signaling domain is CD3ζ.

8. The γδ T cell of any one of claims 1-7, wherein said CAR comprises the binding domain that recognizes the CD70 antigen, followed by a 4-1BB costimulatory domain, followed in turn by a CD3ζ signaling domain.

9. The γδ T cell of any one of claims 1-8, wherein said CAR is encoded by an isolated nucleic acid that is operably linked to a regulatable promoter.

10. The γδ T cell of claim 9, wherein the isolated nucleic acid that encodes the CAR encodes for one or more additional polypeptides.

11. The γδ T cell of claim 10, wherein the one or more additional polypeptides is lymphotoxin beta receptor (LTBR), optionally wherein the LTBR encoded by the isolated nucleic acid has an amino acid sequence as set forth in SEQ ID NO: 27.

12. The γδ T cell of claim 10, wherein the one or more additional polypeptides is a dominant negative (dn) receptor for TGF-beta; preferably wherein the dominant negative receptor for TGF-beta is dnTGFβR2; optionally wherein the dnTGFβR2 encoded by the isolated nucleic acid has an amino acid sequence as set forth in SEQ ID NO: 23.

13. The γδ T cell of claim 10, wherein the one or more additional polypeptides is a truncated form of the human epidermal growth factor receptor (EGFRt); optionally wherein the isolated nucleic acid has an amino acid sequence as set forth in SEQ ID NO: 19.

14. The γδ T cell of claim 10, wherein the one or more additional polypeptides is dominant-negative Fas (dnFas).

15. The γδ T cell of claim 10, wherein the one or more additional polypeptides is membrane-bound IL-12 (mbIL-12).

16. The γδ T cell of any one of claims 10-15, wherein said isolated nucleic acid encodes for at least one cleavage polypeptide sequence; optionally wherein said at least one cleavage polypeptide sequence is a 2A self-cleaving polypeptide sequence selected from T2A, P2A, E2A, and F2A self-cleaving polypeptide sequences.

17. The γδ T cell of claim 16, wherein said 2A self-cleaving polypeptide sequence is a P2A self-cleaving polypeptide sequence; optionally wherein the P2A self-cleaving polypeptide sequence encoded by the isolated nucleic acid has an amino acid sequence as set forth in SEQ ID NO: 8, 77, or 78.

18. The γδ T cell of any one of claims 10-17, wherein the isolated nucleic acid encodes SEQ ID NO: 45, 47, 49, 51, 53, 55, 57, 59, 61, 63, 65, 67, 69, or 71.

19. The γδ T cell of any one of claims 10-17, wherein the isolated nucleic acid encodes SEQ ID NO: 57.

20. The γδ T cell of any one of claims 1-19, further comprising at least one disrupted endogenous gene.

21. The γδ T cell of claim 20, wherein the at least one disrupted endogenous gene is selected from the group comprising or consisting of cytokine inducible SH2-containing protein (CISH), Cbl proto-oncogene B (CBL-B), Zinc finger protein 91 (ZFP91), and Roquin.

22. The γδ T cell of claim 20, wherein the at least one disrupted endogenous gene is CD58 and/or ICAM-1.

23. The γδ T cell of any one of claims 1-22, wherein the γδ T cell exhibits in vitro and/or in vivo tumor cell killing activity against a hematological and/or solid tumor cell that exhibits cell surface expression of the CD70 antigen; preferably wherein the tumor cell killing activity persists for, for about, for at least, or for at least about 6 days to 180 days after first contact with a hematological and/or solid tumor cell that exhibits cell surface expression of the CD70 antigen.

24. The γδ T cell of claim 23, wherein the γδ T cell proliferates in response to contact with the hematological and/or solid tumor cell that exhibits cell surface expression of the CD70 antigen; preferably wherein the γδ T cell proliferation or increased γδ T cell proliferation persists for, for about, for at least, or for at least about, 6 days to 180 days after first contact with the hematological and/or solid tumor cell that exhibits cell surface expression of the CD70 antigen.

25. The γδ T cell of claim 23 or 24, wherein the γδ T cell expresses pro-inflammatory cytokines comprising tumor necrosis factor alpha or interferon gamma after contact with the hematological and/or solid tumor cell that exhibits cell surface expression of the CD70 antigen.

26. The γδ T cell of any one of claims 1-25, wherein the γδ T cell is a δ1 γδ T cell.

27. A pharmaceutical composition comprising a plurality of γδ T cells according to any one of claims 1-26, wherein the plurality comprises at least about 10⁷ γδ T cells, preferably from about 10⁸ γδ T cells to about 10¹¹ γδ T cells; optionally further comprising a pharmaceutically acceptable excipient.

28. The pharmaceutical composition according to claim 27, wherein the plurality comprises a composition that is at least 60%, 80%, or from about 60% or 80% to about 90% or 95% δ1, δ2, δ3, or δ4 γδ T cells, preferably δ1 or δ2 γδ T cells, more preferably δ2⁻ γδ T cells, most preferably δ1 γδ T cells.

29. The method of making a pharmaceutical composition according to claim 27 or 28, wherein the method comprises ex vivo expansion of the γδ T cell(s), wherein the ex vivo expansion is performed before transfection and/or after transfection of the isolated nucleic acid.

30. A method of killing a hematological and/or solid tumor cell that exhibits cell surface expression of a CD70 antigen, the method comprising contacting the hematological and/or solid tumor cell with a tumor cell killing effective amount of the γδ T cell of any one of claims 1-27 or the pharmaceutical composition of claim 27 or 28.

31. The method of claim 30, wherein the method comprises introducing a therapeutically effective amount of the γδ T cell or the pharmaceutical composition into a host organism comprising the hematological and/or solid tumor cell.

32. The method of claim 30 or 31, wherein the method further comprises simultaneously or sequentially administering one or more methods to elevate common gamma chain cytokine(s); optionally wherein the method further comprises administering IL-2 or IL-15, or secreting one or more common gamma chain cytokine(s) from the introduced γδ T cell(s).

33. A method of treating cancer in a subject in need thereof, comprising:
administering a therapeutically effective amount of γδ T cells according to any one of claims 1-26, or a pharmaceutical composition according to claim 27 or 28, wherein the cancer comprises hematological and/or solid tumor cells that exhibit cell surface expression of CD70.

34. The method of claim 33, wherein the method comprises performing a plurality of administrations of the γδ T cells, wherein the interval between the plurality of administrations is at least about a week, preferably at least about 2, 3, 4, 5, 6, 7, 8, or 12 weeks, and/or wherein the plurality of administrations is no more than once every 6 or 12 months.

35. A method of reducing or inhibiting the graft vs. host response to an immune cell administered to a subject in need thereof, comprising administering a therapeutically effective amount of yd T cells according to any one of claims 1-26, or a pharmaceutical composition according to claim 27 or 28.

36. The method of claim 35, wherein the γδ T cells comprise a dual CAR binding to CD70 and another tumor-associated antigen.

37. The method of claim 35 or 36, wherein the method further comprises co-administering the therapeutically effective amount of γδ T cells according to any one of claims 1-26 and a T cell or NK cell comprising a CAR binding to another tumor associated antigen.
